SEQUENCE LISTING (154 sequences)

SEQ ID NO: 1 - protein, 125 aa, Artificial Sequence
  VHH6
DVQFVESGGGSVHAGGSLRLNCATSGYIYSTYCMGWFRQAPGKEREGVAHIYTNSGRTYYADSVKGRFTISQDNAKNTVYLQMNSLKPEDTAIYYCAARPSIRCASFSATEYKDWGQGTQVTVSS

SEQ ID NO: 2 - protein, 10 aa, Artificial Sequence
  VHH6
GYIYSTYCMG

SEQ ID NO: 3 - protein, 17 aa, Artificial Sequence
  VHH6 CDR2
HIYTNSGRTYYADSVKG

SEQ ID NO: 4 - protein, 16 aa, Artificial Sequence
  VHH6 CDR3
RPSIRCASFSATEYKD

SEQ ID NO: 5 - DNA, 375 bp, Artificial Sequence
  VHH6
gatgtgcagttcgtggagtctgggggaggctcggtgcacgctggagggtctctaagactcaactgtgctacctctggatacatttacagtacctattgcatgggatggttccgccaggctccagggaaggagcgtgagggggtcgcacatatttatacgaatagtggacgcacatactatgccgactccgtgaagggccgattcaccatctcccaagacaacgccaagaacacggtgtatctgcaaatgaacagcctgaaacctgaggacacggccatctattactgtgcggcccgtcccagtattaggtgtgcttccttcagcgccaccgaatataaggactggggccaggggacccaggtcaccgtctcctcg

SEQ ID NO: 6 - DNA, 30 bp, Artificial Sequence
  VHH6
ggatacatttacagtacctattgcatggga

SEQ ID NO: 7 - DNA, 51 bp, Artificial Sequence
  VHH6
catatttatacgaatagtggacgcacatactatgccgactccgtgaagggc

SEQ ID NO: 8 - DNA, 48 bp, Artificial Sequence
  VHH6
cgtcccagtattaggtgtgcttccttcagcgccaccgaatataaggac

SEQ ID NO: 9 - protein, 187 aa, Homo sapiens
GSAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 10 - protein, 231 aa, Homo sapiens
GLAPRRCPAQEVARGAGAGDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGAGILQPDPPANITVTAVARNPRWLSVTWQDPHSWNSSFYRLRFELRYRAERSKTFTTWMVKDLQHHAVIHDAWSGLRHVVQLRAQEEFGQGEWSEWSPEAMGTPWTESRSPP

SEQ ID NO: 11 - protein, 8 aa, Artificial Sequence
  Fragment
SSERIDKQ

SEQ ID NO: 12 - protein, 12 aa, Artificial Sequence
  Protein fragment
KDGCFQSCFNEE

SEQ ID NO: 13 - protein, 7 aa, Artificial Sequence
  fragment
LSVTWQD

SEQ ID NO: 14 - protein, 13 aa, Artificial Sequence
  fragment
MVKDLQHHAVIHD

SEQ ID NO: 15 - protein, 6 aa, Artificial Sequence
  fragment
RALRQM

SEQ ID NO: 16 - protein, 18 aa, Artificial Sequence
  fragment
MTTHLILRSFKEFLQSSL

SEQ ID NO: 17 - protein, 8 aa, Artificial Sequence
  fragment
PEGDSSFY

SEQ ID NO: 18 - protein, 13 aa, Artificial Sequence
  fragment
RAQEEFGQGEWSE

SEQ ID NO: 19 - protein, 18 aa, Artificial Sequence
  fragment
LQKKAKNLAITTPDPTTN

SEQ ID NO: 20 - protein, 10 aa, Artificial Sequence
  fragment
RKETCNKSNM

SEQ ID NO: 21 - protein, 12 aa, Artificial Sequence
  fragment
APVPPGEDSKDV

SEQ ID NO: 22 - protein, 21 aa, Artificial Sequence
  fragment
PPGEDSKDVAAPHRQPLTSSE

SEQ ID NO: 23 - protein, 11 aa, Artificial Sequence
  fragment
SSERIDKQIRY

SEQ ID NO: 24 - protein, 10 aa, Artificial Sequence
  fragment
SERIDKQIRY

SEQ ID NO: 25 - protein, 10 aa, Artificial Sequence
  fragment
RIDKQIRYIL

SEQ ID NO: 26 - protein, 12 aa, Artificial Sequence
  fragment
IDKQIRYILDGI

SEQ ID NO: 27 - protein, 11 aa, Artificial Sequence
  fragment
IRYILDGISAL

SEQ ID NO: 28 - protein, 12 aa, Artificial Sequence
  fragment
ALRKETCNKSNM

SEQ ID NO: 29 - protein, 11 aa, Artificial Sequence
  fragment
LRKETCNKSNM

SEQ ID NO: 30 - protein, 12 aa, Artificial Sequence
  fragment
RKETCNKSNMCE

SEQ ID NO: 31 - protein, 21 aa, Artificial Sequence
  fragment
KETCNKSNMCESSKEALAENN

SEQ ID NO: 32 - protein, 15 aa, Artificial Sequence
  fragment
ETCNKSNMCESSKEA

SEQ ID NO: 33 - protein, 24 aa, Artificial Sequence
  fragment
SSKEALAENNLNLPKMAEKDGCFQ

SEQ ID NO: 34 - protein, 11 aa, Artificial Sequence
  fragment
SKEALAENNLN

SEQ ID NO: 35 - protein, 23 aa, Artificial Sequence
  fragment
SKEALAENNLNLPKMAEKDGCFQ

SEQ ID NO: 36 - protein, 11 aa, Artificial Sequence
  fragment
EALAENNLNLP

SEQ ID NO: 37 - protein, 10 aa, Artificial Sequence
  fragment
AENNLNLPKM

SEQ ID NO: 38 - protein, 17 aa, Artificial Sequence
  fragment
AENNLNLPKMAEKDGCF

SEQ ID NO: 39 - protein, 22 aa, Artificial Sequence
  fragment
ENNLNLPKMAEKDGCFQSGFNE

SEQ ID NO: 40 - protein, 8 aa, Artificial Sequence
  fragment
NNLNLPKM

SEQ ID NO: 41 - protein, 11 aa, Artificial Sequence
  fragment
NNLNLPKMAEK

SEQ ID NO: 42 - protein, 23 aa, Artificial Sequence
  fragment
NNLNLPKMAEKDGCFQSGFNEET

SEQ ID NO: 43 - protein, 7 aa, Artificial Sequence
  fragment
AEKDGCF

SEQ ID NO: 44 - protein, 12 aa, Artificial Sequence
  fragment
KDGCFQSGFNEE

SEQ ID NO: 45 - protein, 10 aa, Artificial Sequence
  fragment
QSGFNEETCL

SEQ ID NO: 46 - protein, 8 aa, Artificial Sequence
  fragment
VKIITGLL

SEQ ID NO: 47 - protein, 9 aa, Artificial Sequence
  fragment
VKIITGLLE

SEQ ID NO: 48 - protein, 7 aa, Artificial Sequence
  fragment
IITGLLE

SEQ ID NO: 49 - protein, 11 aa, Artificial Sequence
  fragment
LEYLQNRFESS

SEQ ID NO: 50 - protein, 11 aa, Artificial Sequence
  fragment
EYLQNRFESSE

SEQ ID NO: 51 - protein, 12 aa, Artificial Sequence
  fragment
EYLQNRFESSEE

SEQ ID NO: 52 - protein, 14 aa, Artificial Sequence
  fragment
EYLQNRFESSEEQA

SEQ ID NO: 53 - protein, 22 aa, Artificial Sequence
  fragment
EYLQNRFESSEEQARAVQMSTK

SEQ ID NO: 54 - protein, 11 aa, Artificial Sequence
  fragment
YLQNRFESSEE

SEQ ID NO: 55 - protein, 13 aa, Artificial Sequence
  fragment
YLQNRFESSEEQA

SEQ ID NO: 56 - protein, 11 aa, Artificial Sequence
  fragment
RFESSEEQARA

SEQ ID NO: 57 - protein, 7 aa, Artificial Sequence
  fragment
ESSEEQA

SEQ ID NO: 58 - protein, 11 aa, Artificial Sequence
  fragment
ESSEEQARAVQ

SEQ ID NO: 59 - protein, 8 aa, Artificial Sequence
  fragment
EEQARAVQ

SEQ ID NO: 60 - protein, 8 aa, Artificial Sequence
  fragment
VQMSTKVL

SEQ ID NO: 61 - protein, 8 aa, Artificial Sequence
  fragment
VQMSTKVL

SEQ ID NO: 62 - protein, 7 aa, Artificial Sequence
  fragment
KVLIQFL

SEQ ID NO: 63 - protein, 13 aa, Artificial Sequence
  fragment
IQFLQKKAKNLDA

SEQ ID NO: 64 - protein, 8 aa, Artificial Sequence
  fragment
LQKKAKNL

SEQ ID NO: 65 - protein, 10 aa, Artificial Sequence
  fragment
LQKKAKNLDA

SEQ ID NO: 66 - protein, 19 aa, Artificial Sequence
  fragment
LQKKAKNLDAITTPDPTTN

SEQ ID NO: 67 - protein, 20 aa, Artificial Sequence
  fragment
LQKKAKNLDAITTPDPTTNA

SEQ ID NO: 68 - protein, 22 aa, Artificial Sequence
  fragment
LQKKAKNLDAITTPDPTTNASL

SEQ ID NO: 69 - protein, 15 aa, Artificial Sequence
  fragment
KAKNLDAITTPDPTT

SEQ ID NO: 70 - protein, 12 aa, Artificial Sequence
  fragment
ITTPDPTTNASL

SEQ ID NO: 71 - protein, 8 aa, Artificial Sequence
  fragment
LTKLQAQN

SEQ ID NO: 72 - protein, 8 aa, Artificial Sequence
  fragment
LTKLQAQN

SEQ ID NO: 73 - protein, 11 aa, Artificial Sequence
  fragment
LTKLQAQNQWL

SEQ ID NO: 74 - protein, 13 aa, Artificial Sequence
  fragment
LTKLQAQNQWLQD

SEQ ID NO: 75 - protein, 7 aa, Artificial Sequence
  fragement
QAQNQWL

SEQ ID NO: 76 - protein, 7 aa, Artificial Sequence
  fragment
QDMTTHL

SEQ ID NO: 77 - protein, 9 aa, Artificial Sequence
  fragment
QDMTTHLIL

SEQ ID NO: 78 - protein, 7 aa, Artificial Sequence
  fragment
MTTHLIL

SEQ ID NO: 79 - protein, 7 aa, Artificial Sequence
  fragment
ILRSFKE

SEQ ID NO: 80 - protein, 8 aa, Artificial Sequence
  fragment
ILRSFKEF

SEQ ID NO: 81 - protein, 9 aa, Artificial Sequence
  fragment
ILRSFKEFL

SEQ ID NO: 82 - protein, 7 aa, Artificial Sequence
  fragment
RSFKEFL

SEQ ID NO: 83 - protein, 11 aa, Artificial Sequence
  fragment
RSFKEFLQSSL

SEQ ID NO: 84 - protein, 21 aa, Artificial Sequence
  fragment
HENLYFQGLAPRRCPAQEVAR

SEQ ID NO: 85 - protein, 10 aa, Artificial Sequence
  fragment
GLAPRRCPAQ

SEQ ID NO: 86 - protein, 11 aa, Artificial Sequence
  fragment
GLAPRRCPAQE

SEQ ID NO: 87 - protein, 13 aa, Artificial Sequence
  fragment
GLAPRRCPAQEVA

SEQ ID NO: 88 - protein, 19 aa, Artificial Sequence
  fragment
RCPAQEVARGAGAGDVPPE

SEQ ID NO: 89 - protein, 22 aa, Artificial Sequence
  fragment
AQEVARGAGAGDVPPEEPQLSC

SEQ ID NO: 90 - protein, 14 aa, Artificial Sequence
  fragment
QEVARGAGAGDVPP

SEQ ID NO: 91 - protein, 12 aa, Artificial Sequence
  fragment
EVARGAGAGDVP

SEQ ID NO: 92 - protein, 17 aa, Artificial Sequence
  fragment
VARGAGAGDVPPEEPQL

SEQ ID NO: 93 - protein, 15 aa, Artificial Sequence
  fragment
RGAGAGDVPPEEPQL

SEQ ID NO: 94 - protein, 19 aa, Artificial Sequence
  fragment
DVPPEEPQLSCFRKSPLSN

SEQ ID NO: 95 - protein, 7 aa, Artificial Sequence
  fragment
PPEEPQL

SEQ ID NO: 96 - protein, 9 aa, Artificial Sequence
  fragment
FRKSPLSNV

SEQ ID NO: 97 - protein, 19 aa, Artificial Sequence
  fragment
KSPLSNVVCEWGPRSTPSL

SEQ ID NO: 98 - protein, 13 aa, Artificial Sequence
  fragment
VVCEWGPRSTPSL

SEQ ID NO: 99 - protein, 12 aa, Artificial Sequence
  fragment
VCEWGPRSTPSL

SEQ ID NO: 100 - protein, 15 aa, Artificial Sequence
  fragment
WGPRSTPSLTTKAVL

SEQ ID NO: 101 - protein, 7 aa, Artificial Sequence
  fragment
TTKAVLL

SEQ ID NO: 102 - protein, 12 aa, Artificial Sequence
  fragment
LVRKFQNSPAED

SEQ ID NO: 103 - protein, 11 aa, Artificial Sequence
  fragment
VRKFQNSPAED

SEQ ID NO: 104 - protein, 11 aa, Artificial Sequence
  fragment
QNSPAEDFQEP

SEQ ID NO: 105 - protein, 20 aa, Artificial Sequence
  fragment
AEDFQEPCQYSQESQKFSCQ

SEQ ID NO: 106 - protein, 7 aa, Artificial Sequence
  fragment
DFQEPCQ

SEQ ID NO: 107 - protein, 19 aa, Artificial Sequence
  fragment
CQYSQESQKFSCQLAVPEG

SEQ ID NO: 108 - protein, 18 aa, Artificial Sequence
  fragment
QYSQESQKFSCQLAVPEG

SEQ ID NO: 109 - protein, 11 aa, Artificial Sequence
  fragment
YSQESQKFSCQ

SEQ ID NO: 110 - protein, 7 aa, Artificial Sequence
  fragment
SQKFSCQ

SEQ ID NO: 111 - protein, 14 aa, Artificial Sequence
  fragment
CQLAVPEGDSSFYI

SEQ ID NO: 112 - protein, 10 aa, Artificial Sequence
  fragment
LAVPEGDSSF

SEQ ID NO: 113 - protein, 7 aa, Artificial Sequence
  fragment
PEGDSSF

SEQ ID NO: 114 - protein, 10 aa, Artificial Sequence
  fragment
CVASSVGSKF

SEQ ID NO: 115 - protein, 8 aa, Artificial Sequence
  fragment
ASSVGSKF

SEQ ID NO: 116 - protein, 11 aa, Artificial Sequence
  fragment
LQPDPPANITV

SEQ ID NO: 117 - protein, 10 aa, Artificial Sequence
  fragment
ANITVTAVAR

SEQ ID NO: 118 - protein, 11 aa, Artificial Sequence
  fragment
TAVARNPRWLS

SEQ ID NO: 119 - protein, 8 aa, Artificial Sequence
  fragment
AVARNPRW

SEQ ID NO: 120 - protein, 7 aa, Artificial Sequence
  fragment
VARNPRW

SEQ ID NO: 121 - protein, 10 aa, Artificial Sequence
  fragment
VARNPRWLSV

SEQ ID NO: 122 - protein, 11 aa, Artificial Sequence
  fragment
VARNPRWLSVT

SEQ ID NO: 123 - protein, 12 aa, Artificial Sequence
  fragment
TWQDPHSWNSSF

SEQ ID NO: 124 - protein, 9 aa, Artificial Sequence
  fragment
DPHSWNSSF

SEQ ID NO: 125 - protein, 7 aa, Artificial Sequence
  fragment
YRLRFEL

SEQ ID NO: 126 - protein, 11 aa, Artificial Sequence
  fragment
ELRYRAERSKT

SEQ ID NO: 127 - protein, 9 aa, Artificial Sequence
  fragment
RYRAERSKT

SEQ ID NO: 128 - protein, 14 aa, Artificial Sequence
  fragment
SKTFTTWMVKDLQH

SEQ ID NO: 129 - protein, 17 aa, Artificial Sequence
  fragment
MVKDLQHHAVIHDAWSG

SEQ ID NO: 130 - protein, 24 aa, Artificial Sequence
  fragment
MVKDLQHHAVIHDAWSGLRHVVQL

SEQ ID NO: 131 - protein, 9 aa, Artificial Sequence
  fragment
SGLRHVVQL

SEQ ID NO: 132 - protein, 7 aa, Artificial Sequence
  fragment
LRHVVQL

SEQ ID NO: 133 - protein, 8 aa, Artificial Sequence
  fragment
RHVVQLRA

SEQ ID NO: 134 - protein, 10 aa, Artificial Sequence
  fragment
RAQEEFGQGE

SEQ ID NO: 135 - protein, 11 aa, Artificial Sequence
  fragment
RAQEEFGQGEW

SEQ ID NO: 136 - protein, 19 aa, Artificial Sequence
  fragment
EEFGQGEWSEWSPEAMGTP

SEQ ID NO: 137 - protein, 17 aa, Artificial Sequence
  fragment
GQGEWSEWSPEAMGTPW

SEQ ID NO: 138 - protein, 8 aa, Artificial Sequence
  fragment
WSEWSPEA

SEQ ID NO: 139 - protein, 15 aa, Artificial Sequence
  fragment
WSEWSPEAMGTPWTE

SEQ ID NO: 140 - protein, 20 aa, Artificial Sequence
  fragment
WSEWSPEAMGTPWTESRSPP

SEQ ID NO: 141 - protein, 7 aa, Artificial Sequence
  fragment
SEWSPEA

SEQ ID NO: 142 - protein, 14 aa, Artificial Sequence
  fragment
SEWSPEAMGTPWTE

SEQ ID NO: 143 - protein, 19 aa, Artificial Sequence
  fragment
SEWSPEAMGTPWTESRSPP

SEQ ID NO: 144 - protein, 12 aa, Artificial Sequence
  fragment
WSPEAMGTPWTE

SEQ ID NO: 145 - protein, 15 aa, Artificial Sequence
  fragment
PEAMGTPWTESRSPP

SEQ ID NO: 146 - protein, 7 aa, Artificial Sequence
  fragment
MGTPWTE

SEQ ID NO: 147 - protein, 125 aa, Artificial Sequence
  hVHH6
EVQFVESGGGLVQPGGSLRLSCATSGYIYSTYCMGWFRQAPGKEREGVAHIYTNSGRTYYADSVKGRFTISQDNAKNTVYLQMNSLRAEDTAVYYCAARPSIRCASFSATEYKDWGQGTLVTVSS

SEQ ID NO: 148 - protein, 125 aa, Artificial Sequence
  hVHH6
EVQFVESGGGLVQPGGSLRLSCATSGYIYSTYSMGWFRQAPGKEREGVAHIYTNSGRTYYADSVKGRFTISQDNAKNTVYLQMNSLRAEDTAVYYCAARPSIRSASFSATEYKDWGQGTLVTVSS

SEQ ID NO: 149 - protein, 10 aa, Artificial Sequence
  hVHH6
GYIYSTYCMG

SEQ ID NO: 150 - protein, 10 aa, Artificial Sequence
  hVHH6
GYIYSTYSMG

SEQ ID NO: 151 - protein, 17 aa, Artificial Sequence
  hVHH6
HIYTNSGRTYYADSVKG

SEQ ID NO: 152 - protein, 16 aa, Artificial Sequence
  hVHH6
RPSIRCASFSATEYKD

SEQ ID NO: 153 - protein, 16 aa, Artificial Sequence
  hVHH6
RPSIRSASFSATEYKD

SEQ ID NO: 154 - protein, 113 aa, Homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARYFDYWGQGTLVTVSS

(19) United States
(12) Patent Application Publication
(10) Pub. No.: US 2021/0301012 A1
(43) Pub. Date: Sep. 30, 2021

(54) Proteins and Uses

(71) Applicant: UCB Biopharma SPRL, Brussels (BE)

(72) Inventors: Michael John Wright, Slough, Berkshire (GB); Ralph Adams, Slough, Berkshire (GB); Rebecca Burnley, Slough, Berkshire (GB); Anna Ettorre, Slough, Berkshire (GB)

(21) Appl. No.: 16/342,800

(22) PCT Filed: Oct. 23, 2017
(86) PCT No.: PCT/EP2017/076948

§ 371 Date: Apr. 17, 2019

(30) Foreign Application Priority Data
Oct. 24, 2016 (GB) 1617924.4

(51) Int. Cl. C07K 16/24

(52) U.S. Cl. CPC C07K 16/248; C07K 2317/565; C07K 2317/32; C07K 2319/70; C07K 2317/92

(57) ABSTRACT

The invention is in the field of protein:protein interactions, in particular the stabilisation of protein:protein interactions. Binding proteins are provided for stabilising the interactions, together with compositions comprising the binding proteins. Methods using the binding proteins are also provided, including targeting of cells and also medical uses.

### FIELD OF THE INVENTION

The present invention relates to stabilising transient protein:protein interactions using binding proteins. Pharmaceutical compositions comprising the binding proteins are also provided. The invention also relates to use of the binding proteins, including use in delivering cargo to a target cell and use in cross-linking target cells. Various methods are further provided, including methods of specifically targeting cells using the binding protein, methods of switching on a biological effect and methods of cross-linking two cells.

### BACKGROUND OF THE INVENTION

In cancer, most tumour associated antigens are also expressed on normal peripheral tissues and cells, albeit at disparate density. It is commonly believed that the dual targeting of two antigens on the same cell leads to improved target selectivity over normal tissues that express only one or low levels of both antigens.

One means of targeting two antigens on a tumour cell is via a bispecific antibody (i.e. with one arm of the antibody recognising one target antigen and the second arm of the antibody recognising a second target antigen). However, as shown in Mazor et al mAbs, 7, 461-469, dual targeting by bispecific antibodies is not always sufficient to promote efficient target selectivity. Bispecific antibodies may still recognise cells expressing only one of the two target antigens. Mazor et al reported that the affinity of the individual arms of the antibody, as well as avidity and valence, play an important role in targeting. For example, a bispecific antibody composed of an anti-CD4 arm and an anti-CD70 arm recognised cells expressing both CD4 and CD70, as well as CD4 alone. Affinity-reduced variants of the CD4 arm were prepared by introducing mutations into the CDRs. These variants exhibited improved selectively; binding to cells expressing CD4 alone was reduced. However, in generating such bispecific antibodies extensive engineering is required. Bispecific antibodies are also inherently specific for their particular target antigens. Different bispecific antibodies are thus needed to target different antigen combinations.

Another technique for recognising multiple antigens on a target cell is described in WO 2013/104804. Here, a targeting moiety for a first antigen “A” is linked to a non-active fragment of a functional domain. A second targeting moiety for antigen “B” is then linked to a second non-active fragment of the functional domain. When both antigens are present on the cell surface, dimerization of the two fragments results in formation of a functional domain.

One fragment may be a V_{L} domain of an antibody and the other fragment may be a V_{H} domain of an antibody thus resulting in an scFv when both antigens are present on the target cell. There are, however, also downsides to this approach in that V_{H} and V_{L} by themselves can have stability issues and the system may be partially leaky. Separate V_{H} and V_{L} domains may also be prone to aggregation.

There exists a need in the art to provide improved means to specifically identify and target cells (e.g. immune cells and tumour cells) with minimal off target effects.

The present inventors have identified a new system with utility, amongst other things, in specifically targeting multiple antigens on cells of interest. The system harnesses transient protein:protein interactions (PPIs), and in particular uses junctional binding proteins which recognise two components of a transient PPI.

An example of a transient PPI, which plays a fundamental role in the patho-physiology of several diseases, is the interaction between IL-6 and its specific receptor gp80, (also known as CD126). IL-6 binds to gp80 to form a heterodimer; this first step is characterized by fast association and dissociation phases (Ward et al. The Journal of biological chemistry 271, 20138-20144 (1996)). The IL-6−gp80 complex then can bind to gp130 to form a heterotrimer, which in turn dimerizes to create the active hexameric complex responsible for key downstream signaling events (Boulanger et al Science 300, 2101-2104 (2003)). IL-6 can signal in cis or trans, depending on whether gp80 is cell membrane-expressed or in a soluble form generated by shedding (Mullberg et al. European journal of immunology 23, 473-480 (1993)). Both forms of gp80 are active and bind IL-6 (Rose-John et al Journal of leukocyte biology 80, 227-236 (2006)). In vitro data suggest that the trans-signaling pathway is preferentially activated during inflammation (Rose-John et al. International journal of biological sciences 8, 1237-1247 (2012)). Unlike gp130, which is expressed in a variety of cells, gp80 is expressed mainly on the membrane of hepatocytes and immune cells (Rose-John et al Journal of leukocyte biology 80, 227-236 (2006)). Neither IL-6 nor gp80 binds to gp130, indicating that IL-6 is presented by gp80 in the appropriate conformation to then recruit gp130 (Rose-John et al Journal of leukocyte biology 80, 227-236 (2006)).

IL-6 mediates an array of immune responses including lymphocyte trafficking, proliferation and differentiation of T cells, antibody production from B cells, liver regeneration, expansion/differentiation of bone marrow progenitors and the broad regulation of inflammatory response (Galun, E. & Rose-John, S Methods in molecular biology 982, 59-77 (2013) and Mihara et al Clin Sci (Lond) 122, 143-159 (2012)). IL-6 is a key mediator in a variety of diseases, including cancer and autoimmunity (Hunter, C. A. & Jones, S. A. Nature immunology 16, 448-457 (2015)). As such, IL-6 has been targeted directly (IL-6 blocking antibodies) or indirectly (gp80 blocking antibodies; gp130-Fc) for therapeutic interventions against autoimmune disorders and cancer (Rose-John et al Expert opinion on therapeutic targets 11, 613-624 (2007), Calabrese, L. H. & Rose-John, S. Nature reviews. Rheumatology 10, 720-727 (2014), Shaw, S. et al. Discovery and characterization of olokizumab: a humanized antibody targeting interleukin-6 and neutralizing gp130-signaling. mAbs 6, 774-782 (2014), Atreya, R. et al, Nature medicine 6, 583-588 (2000), Nishimoto, N. et al. Blood 106, 2627-2632 (2005) and Milagre, C. S. et al. Cancer research 75, 1255-1264 (2015)).

### SUMMARY OF THE INVENTION

The present invention provides:

A binding protein specific for a junctional epitope created by a transient protein:protein interaction, which binding protein stabilises the protein:protein interaction.

The present invention also provides an isolated polynucleotide encoding one or both chains of an antibody or fragment thereof of the invention, a vector comprising the polynucleotide, a host cell comprising the vector and a method of producing an antibody or fragment thereof of the invention, said method comprising culturing the host cell of the invention in a medium to produce the antibody and collecting the antibody or fragment thereof from the culture.

The invention further provides a pharmaceutical composition comprising a binding protein of the invention and a pharmaceutically acceptable carrier or diluent.

The invention also provides use of a binding protein of the invention in delivering cargo to a target cell.

The invention further provides a method of delivering cargo to a target cell, comprising:

(1) administering two targeting molecules, wherein:
the first targeting molecule comprises a first targeting portion which binds specifically to a first target molecule on the target cell and wherein the targeting portion is linked to the first protein component of a transient protein:protein interaction; and
the second targeting molecule comprises a second targeting portion which binds specifically to a second target molecule on the target cell and wherein the targeting portion is linked to the second protein component of the transient protein:protein interaction;

(2) administering a binding protein of the invention specific for a junctional epitope created by the transient protein:protein interaction, which binding protein is linked to the cargo.

The invention additionally provides use of a binding protein of the invention in switching on a biological effect.

The invention further provides a method of switching on or off a biological effect, comprising:
(1) administering two targeting molecules, wherein:
   the first targeting molecule comprises a first targeting portion which binds specifically to a first target molecule on the target cell and wherein the targeting portion is linked to the first protein component of a transient protein:protein interaction; and
   the second targeting molecule comprises a second targeting portion which binds specifically to a second target molecule on the target cell and wherein the targeting portion is linked to the second protein component of the transient protein:protein interaction; and
(2) administering a binding protein of the invention specific for a junctional epitope created by the transient protein:protein interaction, wherein administration of the binding protein switches on or off the biological effect.

Furthermore, the invention provides a method of amplifying, decreasing or modifying of a biological effect, comprising:
(1) administering two targeting molecules, wherein:
   the first targeting molecule comprises a first targeting portion which binds specifically to a first target molecule on the target cell and wherein the targeting portion is linked to the first protein component of a transient protein:protein interaction; and
   the second targeting molecule comprises a second targeting portion which binds specifically to a second target molecule on the target cell and wherein the targeting portion is linked to the second protein component of the transient protein:protein interaction; and
(2) administering a binding protein specific for a junctional epitope created by the transient protein:protein interaction, wherein the binding protein is as defined above and wherein administration of the binding protein amplifies, decreases or modifies the biological effect.

The invention further provides use of a binding protein of the invention in cross-linking target cells.

The invention additionally provides the following methods of cross-linking target cells:

(A) A method of cross-linking two target cells using a binding protein, wherein the first target cell expresses a first target molecule and the second target cell expresses a second target molecule, and wherein the method comprises:
(1) administering two targeting molecules, wherein:
   the first targeting molecule comprises a first targeting portion which binds specifically to the first target molecule on the first target cell and wherein the targeting portion is linked to the first protein component of a transient protein:protein interaction; and
   the second targeting molecule comprises a second targeting portion which binds specifically to the second target molecule on the second target cell and wherein the targeting portion is linked to the second protein component of the transient protein:protein interaction; and
(2) administering a binding protein of the invention specific for a junctional epitope created by the transient protein:protein interaction.

(B) A method of cross-linking two target cells, wherein the first target cell expresses a first and second target molecule and the second target cell expresses a third target molecule, and wherein the method comprises:
(1) administering two targeting molecules, wherein:
   the first targeting molecule comprises a first targeting portion which binds specifically to the first target molecule on the first target cell and wherein the targeting portion is linked to the first protein component of a transient protein:protein interaction; and
   the second targeting molecule comprises a second targeting portion which binds specifically to the second target molecule on the first target cell and wherein the targeting portion is linked to the second protein component of the transient protein:protein interaction; and
(2) administering a third targeting molecule comprising a third targeting portion which binds specifically to the third target molecule on the second target cell, wherein the targeting portion is linked to a binding protein of the invention specific for a junctional epitope created by the transient protein:protein interaction.

(C) A method of cross-linking two target cells, wherein the first target cell expresses a first and second target molecule and wherein the second target cell is engineered to present a binding protein on its surface, and wherein the method comprises administering two targeting molecules, where:
the first targeting molecule comprises a first targeting portion which binds specifically to the first target molecule on the first target cell and wherein the targeting portion is linked to the first protein component of a transient protein:protein interaction; and
the second targeting molecule comprises a second targeting portion which binds specifically to the second target molecule on the first target cell and wherein the targeting portion is linked to the second protein component of the transient protein:protein interaction;
   wherein the binding protein is as defined above.

Furthermore, the invention provides a method of treatment of the human or animal body, comprising administering a binding protein of the invention.

In particular, the invention provides a method of tissue regeneration, comprising administering a binding protein of the invention, wherein the protein:protein interaction is the IL-6/gp80 interaction.

The invention also provides a method of expanding hematopoietic progenitor cells or hepatocyte-like progenitor cells using a binding protein of the invention, wherein the protein:protein interaction is the IL-6/gp80 interaction.

The invention also provides a method of producing an antibody specific for a junctional epitope created by a transient protein:protein interaction, said method comprising immunising an animal with a fusion protein of the stabilised transient protein:protein interaction and selecting an antibody which interacts with the stabilised complex of the protein:protein interaction but not with each individual component of the protein:protein interaction in the absence of the other.

Finally, the invention provides use of a binding protein of the invention in screening for a substance which modulates signalling through the protein:protein interaction.

### DESCRIPTION OF THE FIGURES

FIG. 1 exemplifies a two step targeting modality using a radiolabelled junctional antibody (oval attached to a star) with cancer cells expressing antigens A and B (lower diagrams) in comparison with a bispecific antibody (upper diagrams). In the two step targeting modality, the cells are first targeted with antibodies (each represented with four ovals), which are highly specific for the target antigens and which are fused to the components of the interaction recognised by the junctional antibody (the cut out circle and the rectangle). For example, cells are targeted with an antibody targeting antigen A fused to one protein component of the transient protein/protein interaction recognised by the junctional antibody. The cells are also targeted with a second antibody targeting antigen B fused to the second protein component of the transient protein/protein interaction recognised by the junctional antibody. This is followed by administration of the radiolabeled junctional antibody, which cross-links the two components. This system allows for specific targeting of cells expressing both antigen A and antigen B. In contrast, the bispecific antibody as shown in the upper diagrams targets cells expressing antigen A alone or antigen B alone to some extent.

FIG. 2 shows how a junctional antibody could be used to switch on/off a molecular bispecific functional effect (using antibody fusions as described for FIG. 1). Again, the two-step system with administration of the antibody fusions followed by the junctional antibody allow for a level of flexibility which cannot be achieved with a bispecific antibody. Addition of the junctional antibody switches on the biological effect.

FIG. 3 illustrates how a junctional antibody can be used to cross-link two target cells. Once again, the junctional antibody is administered after the two targeting molecules (the antibody fusions), resulting in functional cross linking of the two target cell populations. The two-step targeting approach allows for greater control than with a bispecific antibody.

FIG. 4 shows a more complex three-antigen scenario. Such a system could be used to bring two cells together, e.g. target cell with antigens A and B and an effector for cell killing (with the junctional antibody fused to an antibody which targets an antigen on the effector cell). Extra specificity is provided by requiring the two targeting antibodies to recognise the two individual antigens (A and B) on the target cell before the junctional antibody can engage and attract the effector cell. This essentially combines the models of FIGS. 1 and 3.

FIG. 5 shows the specific targeting of e.g. cancer cells (left hand side) by an engineered effector cell (right hand side) benefitting from the junctional antibody high specificity with a dual targeting approach (to antigens A and B on the cancer cell). This example expands the use of the junctional antibody to again only specifically target a cell population with two antigens. The effector cell may be patient derived or an engineered human cell line that expresses the junctional antibody as a cell membrane fusion protein. These cells can be administered to a patient and can be long lived. Only on the administration of the two antibody-fusions would these effector cells find their junctional epitope and lead to cell killing.

FIG. 6 shows an example of how a junctional antibody (labelled JEA) can be used to cross-link two antigens (Ag A and Ag B) on the surface of a single cell via antibody fusions (Ab A with component A and Ab B with component B). There are at least four individual protein protein interactions (PPIs) described which will determine how effectively the two antigens are cross-linked. Modulation of the interactions allows for fine tuning of the system.

FIG. 7 shows results of the preliminary assays to prove that VHH6 only recognized IL-6−gp80 complex. (a) ELISA: FusionIL-6, IL-6 or gp80 were immobilized overnight on plates. VHH6 binds to FusionIL-6, but not to IL-6 or gp80 alone. When the plates were coated with IL-6 and VHH6 added with gp80, or VHH6 was added with IL-6 (on gp80-coated plates), the absorbance was comparable to FusionIL-6. y-axis: Absorbance 630 nm; x-axis: samples. (b) MicroScale Thermophoresis (MST): IL-6 and gp80 were labelled with NT647. A binding curve was apparent only when different concentrations of VHH6 were incubated with a mixture of IL-6-NT647+80 or gp80-NT647+IL-6 (bottom two panels), but not when either IL-6-NT647 or gp80-NT647 alone were probed. y-axis: thermophoresis intensity; x-axis: VHH6 concentration (nM). (c) Bio-Layer Interferometry (Octet): VHH6 was captured through its His-tag onto the Ni-tips and solutions of FusionIL-6, IL-6, gp80 and IL-6+gp80 were probed. A change of the interference pattern for VHH6 was recorded only for FusionIL-6 or IL-6+gp80 solutions. y-axis: wavelength shift (nm); x-axis: time (s). (d) SEC isolation of VHH6 in complex with IL-6 and gp80. Solutions of IL-6, gp80 and VHH6 alone and in combination. SEC on IL-6+VHH6, gp80+VHH6 give two separate peaks corresponding to the elution time of the single loaded components, while the elution time of VHH6−IL-6−gp80 was compatible with the elution of the trimeric complex when compared to the elution of the dimeric complex IL-6−gp80 (respectively, 9.378 min vs. 9.556 min). y-axis: Absorbance at 280 nm; x-axis: time (min).

FIG. 8 shows the crystal structure of VHH6−IL-6−gp80 complex. (a) Crystal structure of VHH6 bound simultaneously to IL-6 and gp80. The epitope covers a total surface area of 924 Å², with the interface between VHH6 and IL-6 contributing 414 Å² and the interface between VHH6 and gp80 contributing 510 Å². Superimpositions of free IL-6 (1ALU) and free gp80 (1N26) backbone carbon alpha atoms over the equivalent atoms of VHH6−IL-6−gp80 showed no significant shifts in conformation with root mean squared deviation (r.m.s.d.) values of 1.2 Å and 1.5±0.1 Å, respectively. The Figure on the right shows the complex rotated by 90° around x-axis. (b) CDR1 and CDR3 make contacts with both IL-6 and gp80 whereas CDR2 contacts only gp80. (c) At the gp80 interface, CDR3 residue Ser101 and CDR2 residues Ser30 and Thr31 (both main-chain oxygen atoms) form a hydrogen bond network with Ser228 and Arg231; main chain oxygen atom of CDR2 residue Asn54 forms a hydrogen bond with the main chain nitrogen atom of Asp221; and framework residue Asn74 forms a hydrogen bond with the main chain oxygen atom of His256. (d) At the interface with IL-6, CDR3 residues Ser101 and Ile102 (main-chain nitrogen atom) hydrogen bond with Gln183; CDR3 residue Lys113 forms a salt bridge with Glu80; there is an aliphatic interaction between Tyr27 and Glu23; and CDR1 residue Tyr32 form hydrogen bonds with Ser22 (see also FIG. 13 for more details).

FIG. 9 shows HDX-MS of VHH6−IL-6−gp80 complex and confirms the junctional epitope identified by crystallography and indicated reduced structural dynamics at the IL-6−gp80 interface. HDX-MS was applied to map the epitope of the VHH by comparing the HDX for a purified IL-6−gp80 complex with that for a purified VHH6−IL-6-gp80 complex. After the exchange reaction, the proteins were digested by pepsin and the resulting peptides analyzed by LC/MS. Sequence coverage of 99.6% was achieved for gp80 and 94.1% for IL-6. No data were obtained for the residues 179-184 (RALRQM; SEQ ID NO: 15) at the C-terminus of IL-6, also expected to interact directly with the VHH. HDX-MS data are represented on (a) the crystal structure of VHH6−IL-6−gp80 and (b) the sequences of IL-6 and gp80. Peptides with differential-deuterium uptake in the presence and absence of the VHH6 with p<0.01 (Students' t-test) for ≥2 time-points are defined as significant. (a) Left hand image: arrow indicates loop rigidified upon VHH binding; right hand image: arrows indicate regions involved in gp80-IL-6 interface.

(b) Regions showing a decrease in HDX-MS in the presence of VHH6 are in bold. Regions showing an increase in HDX are lighter in colour.

FIG. 10 shows that binding of VHH6 to IL-6−gp80 complex changes the dissociation rate of the complex but does not affect the binding of IL-6−gp80 complex to gp130. (a) Qualitative SPR experiment was performed immobilizing IL-6 on a CMS chip and injecting gp80. The curves represent a typical sensorgram of gp80 on IL-6; and the sensorgram resulting from gp80 on IL-6 with VHH6 injected at the beginning of the dissociation phase for gp80, showing an inhibition of gp80 dissociation curve (y-axis: RU; x-axis: time (s)). (b) Quantitative SPR was performed immobilizing IL-6 on a CMS chip and injecting gp80. Different concentrations of gp80 were tested in the absence or in the presence of an excess of VHH6 (see Materials on line). k_{d} (k_{off}) are reported above each sensorgram (y-axis: RU; x-axis: time (s)) (k_{d} and k_{off} are used interchangeably herein). (c) gp130 was immobilized on a CMS chip. A fixed excess concentration of IL-6 and differing concentrations of gp80 in the presence or absence of VHH6 were tested. IL-6-gp80 complex binds gp130 with ka (kₒₙ) of 3.6×10⁵ M⁻¹ s⁻¹ and kd (k_{off}) 0.037 s⁻¹ while the VHH6−IL-6−gp80 complex binds gp130 with ka (kₒₙ) of 4.7×10⁵ M⁻¹ s⁻¹ and kd (k_{off}) 0.029 s⁻¹ (y-axis: RU; x-axis: time (s)). The data confirmed that are no significant differences neither in the association or the dissociation phase of IL-6−gp80 complex to gp130 upon addition of VHH6.

FIG. 11 shows that stabilization of the complex IL-6−gp80 by VHH6 increases the internalization of IL-6-NT647 over-time and promotes higher and sustained STAT3 phosphorylation signal in HUVEC cells. HUVEC cells were treated with IL-6+80 and VHH6+IL-6+gp80 or IL-6-NT647+gp80 and VHH6+IL-6-NT647+80. Both pSTAT3 and IL-6-NT647 fluorescent signal were quantified at different time points using the ‘spot total intensity per object’ parameter. Three different replicates were performed for each time point (statistical significance: *=p≤0.05; **=p≤0.01 and ***=p≤0.001). (a) A representative experiment showing pSTAT3 signal and nuclear translocation at 30 min. (Scale bar=20 μm). (b) A representative time-course experiment showing pSTAT3 signal at 30, 180 and 360 min. (Scale bar=100 μm). (c) Statistical analysis of pSTAT3 signal over-time showing that VHH6 promotes and increased and sustained pSTAT3 signal. y-axis: pSTAT3 fluorescence; x-axis: time (min).

IL-6 labelled with NT647 was used to track over-time internalization of the IL-6-gp80 complex. VHH6 significantly increased the rate of IL-6-NT647 accumulation in vesicles in a time-dependent manner. (d) A representative time-course experiment showing IL-6NT647 signal at 30, 180 and 360 min. (Scale bar=20 μm). (e) Accumulation of IL-6-NT647 co-localized with LAMP-1 (t=360 min; Scale bar=20 μm). (f) Statistical analysis of IL-6-NT647 vesicle intensity at 15, 30, 90, 180 and 360 min. y-axis: IL-6-NT647 vesicle intensity; x-axis: treatments grouped per time point.

FIG. 12 shows transcriptomic analysis of HUVEC cells treated with VHH6−IL-6-gp80 and confirms selective up-regulation of pro-inflammatory genes. Three different batches of HUVEC cells were analyzed at 30, 180 and 360 min. VHH6−IL-6−gp80 (sample) were compared to IL-6−gp80 (control). For genes which showed downregulation or upregulation, the fold changes from each batch of HUVEC cells at three different time points were plotted and clustered according to cellular function. (a) cytokines, chemokines and chemokine receptors; (b) transcription factors and cytokine regulators; (c) kinases. y-axis: fold change; x-axis: time (min). Statistical significance: *=p≤0.05; **=p≤0.01 and ***=p≤0.001.

FIG. 13 shows differential hydrogen bonding in the two copies of the crystallographic asymmetric unit. There are two copies of the VHH6−IL-6−gp80 complex in the crystallographic asymmetric unit. In one copy, IL-6 residue Ser22 forms a hydrogen bond with VHH6 residue Tyr32 (left), and in the second copy, Ser22 forms a hydrogen bond with VHH6 residue Tyr27 (right).

FIG. 14 shows a model of the crystal structure of VHH6−IL-6−gp80 superimposed with gp130. Superimposition of IL-6 and gp80 from the signaling complex, IL-6−gp80-gp130 (PDB code 1P9M), showed low r.m.s.d. values of 1.4±0.2 Å and 1.35±0.05 Å respectively, indicating that VHH6 holds IL-6 and gp80 together in a form which is able to bind gp130.

FIG. 15 shows that stabilization of the complex IL-6−gp80 by VHH6 promoted higher and sustained STAT3 phosphorylation signal in HUVEC cells comparable to FusionIL-6 fusion protein. HUVEC cells were treated with FusionIL-6, IL-6+gp80 and VHH6+IL-6+gp80. STAT3 phosphorylation signal was quantified at different time points using the ‘spot total intensity per object’ parameter. Statistical analysis of pSTAT3 signal from three replicates was performed for each time point (statistical significance: *=p≤0.05; **=p≤0.01 and ***=p≤0.001). Unlike IL-6−gp80, in the presence of VHH6 or when FusionIL-6 was added to the culture, pSTAT3 was increased at earlier (30 min) and later time points (180 min and 360 min). y-axis: pSTAT3 fluorescence expressed as spot total intensity per object; x-axis: time (min).

FIG. 16 shows that transcriptomic analysis of HUVEC cells treated with VHH6−IL-6-gp80 confirmed selective up-regulation of pro-inflammatory genes. Three different batches of HUVEC cells were analyzed at 30, 180 and 360 min. VHH6−IL-6−gp80 (sample) were compared to IL-6−gp80 (control). Data were analysed using the RT2 Profiler PCR array web based data analysis template v3.5 (http://pcrdataanalysis.sabiosciences.com/pcr/arrayanalysis) and changes in gene expression changes were calculated using the ΔΔCₜ method with normalization of the raw data to housekeeping genes. Data from samples were compared to controls (84 genes from 3 samples and 3 control) and a heat map was generated using Genedata's Analyst software. Gene regulation for each sample is expressed as fold change when compared to control.

FIG. 17 shows the correlation between kₒₙ, k_{off} and K_{D} values. In particular, the bold diagonal line represents a K_{D} of 10 nM. Anything to the right of the line has a higher (i.e. weaker) equilibrium dissociation constant than 10 nM. Anything to the left of the line has a lower (i.e. stronger/improved) dissociation constant than 10 nM. The diagonal line demonstrates how a high off rate (a high k_{off} value, e.g. 10⁰ s⁻¹) and high on rate (a high kₒₙ value e.g. 10⁸ M⁻¹s⁻¹), or a low off rate (a low k_{off} value e.g. 10⁻⁶ s⁻¹) and a low on rate (a low kₒₙ value e.g. 10² M⁻¹s⁻¹) can both result in a dissociation constant of 10 nM.

FIG. 18 shows the alignment of Llama VHH6 sequence with humanized grafts of VHH6 variable heavy chain using IGHV3-74 human germline as the acceptor framework. CDRs are shown in bold/underlined. Donor residues are shown in bold/italic and are highlighted: F4, T24, F37, E44, R45, E46, G47, A49, Q72, V79 and A98. Modifications to CDRs are shown in bold/underlined and are highlighted: C33S, C104S.

### DESCRIPTION OF THE SEQUENCE LISTING

SEQ ID NOs: 1-8 show the CDR and heavy chain variable region amino acid and nucleotide sequences of the VHH6 antibody.

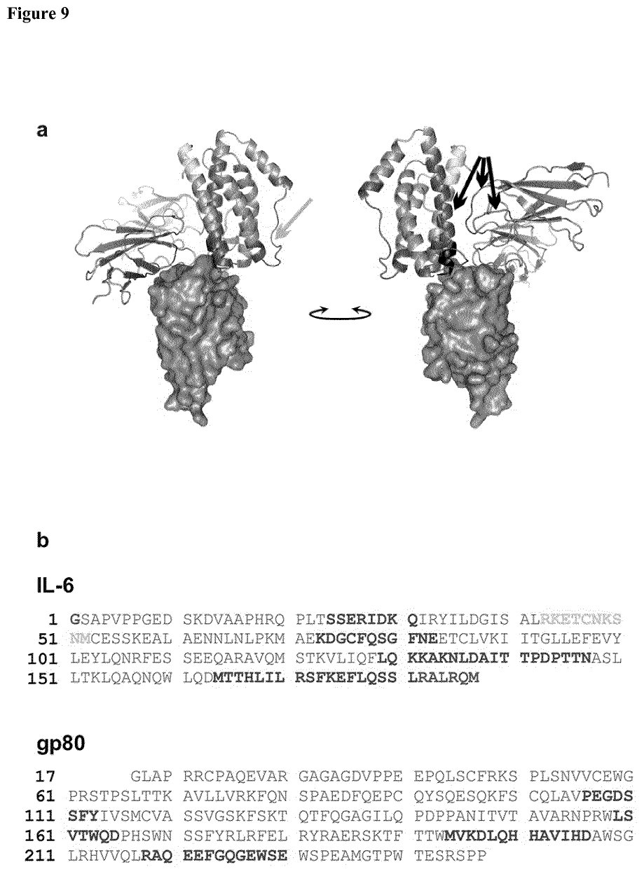

SEQ ID NO: 9 shows the IL-6 sequence from FIG. 9 (full IL-6 sequence).

SEQ ID NO: 10 shows the gp80 sequence from FIG. 9 (modified gp80 sequence as used in the Examples).

SEQ ID NOs: 11-146 show sequences featured in the Examples.

SEQ ID Nos: 147-153 show the CDRs and heavy chain variable region amino acid sequences of the humanized VHH6 antibodies.

SEQ ID NO: 154 shows the sequence of the human IGHV3-74 IGHJH4 acceptor framework.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an amino acid sequence” includes two or more such sequences, and the like.

All publications, patents and patent applications cited herein, whether supra or infra, are hereby incorporated by reference in their entirety.

### Binding Protein Specific for a Junctional Epitope Created by a Transient Protein:Protein Interaction

The present invention relates to a binding protein specific for a junctional epitope created by a transient protein:protein interaction, which binding protein stabilises the protein:protein interaction.

The concept of “transient protein:protein interactions” (or PPIs) is well known in the art. See, for example, Nooren and Thornton (2003) The EMBO Journal, 22, 3486-3492 and Perkins et al (2010) Structure. 18, 1233-1243. PPIs can be distinguished based on the lifetime of the complex. In contrast to a permanent interaction which is usually very stable and thus exists in its complexed form, a transient interaction associates and dissociates in vivo. A complex qualifies as transient if it readily undergoes changes in oligomeric state.

PPIs may be classified based on the equilibrium dissociation constant (K_{D}) which takes account of the rate of the complex dissociation (k_{off}) and the rate of the complex association (kₒₙ), where:

$${K}_{D} = \frac{{k}_{o f f}}{{k}_{o n}}$$

K_{D} is expressed in M, k_{off} is expressed in s⁻¹ and kₒₙ is expressed in M⁻¹s⁻¹.

Methods of investigating protein:protein interactions include yeast two hybrid screens (e.g. repeated yeast two hybrid screens, where transient interactions are often detected in only one screen, whereas permanent interactions are detected in multiple screens) and tandem affinity purification in conjunction with mass spectrometry. Such techniques are discussed in Perkins et al (2010) Structure. 18, 1233-1243. Another technique used to characterise protein:protein interactions is NMR (see Vonigradova and Qin (2012), Top Curr Chem, 326, 35-45).

In the present invention, it is preferable that surface plasmon resonance (SPR; Biacore) is used in characterising the binding kinetics of the PPI. SPR is described in the Examples, and the protocols/equipment as used in the Examples may be employed in the present invention. In the invention, the SPR experiments are typically carried out at 25° C.

In the present invention, the transient PPI preferably has a weak affinity (equilibrium dissociation constant; K_{D}). The weak K_{D} is typically characterised by a high (fast) dissociation rate (k_{off}). The PPI, in the context of the present invention, may be characterised by both a low association rate (kₒₙ) and a high dissociation rate (k_{off}). In other words, the two protein components of the PPI preferably have low complex formation in the absence of the binding protein, and any complexes formed dissociate quickly. This high dissociation rate and low association rate makes the PPI suited to the methods described below. In some scenarios, the protein:protein interaction may also have a higher association rate, but a fast dissociation rate still means the complex dissociates rapidly, thus still making the protein:protein interaction suited to the methods described below. In other situations, it is also possible that the complex has a low dissociation rate but a low association rate means complex formation happens infrequently.

In the absence of the binding protein, the protein components of the transient PPI may have undetectable binding to one another or may have insufficient interaction to result in the downstream function of the complex. For example, the two protein components of the PPI may have undetectable binding using techniques such as gel filtration (size exclusion chromatography), ELISA or surface plasmon resonance. These techniques are discussed further below and are utilised in the Examples.

With regards to lack of downstream function, this would be determined by the nature of the PPI. The present Examples utilise the IL-6/gp80 interaction, which binds to gp130 (see above) and leads to a series of phosphorylation events, ultimately culminating in the phosphorylation of STAT3. gp130 binding or STAT3 phosphorylation could therefore be used as a measure of interaction of the IL-6 and gp80. For example, whilst the IL-6/gp interaction already has a fast off rate, the binding interface of the proteins could be engineered (residues mutated) to increase the off rate and/or decrease the on rate and/or increase the dissociation constant of the interaction (a higher dissociation constant means weaker binding). Binding might then become undetectable using the above mentioned techniques or might reduce or eliminate downstream signalling.

The protein components of the transient PPI may also have insufficient interaction in the absence of the binding protein to see an end result in a patient using the methods described below.

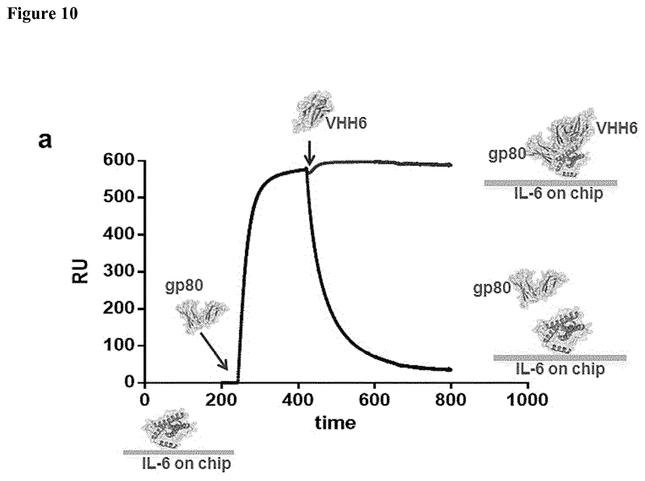

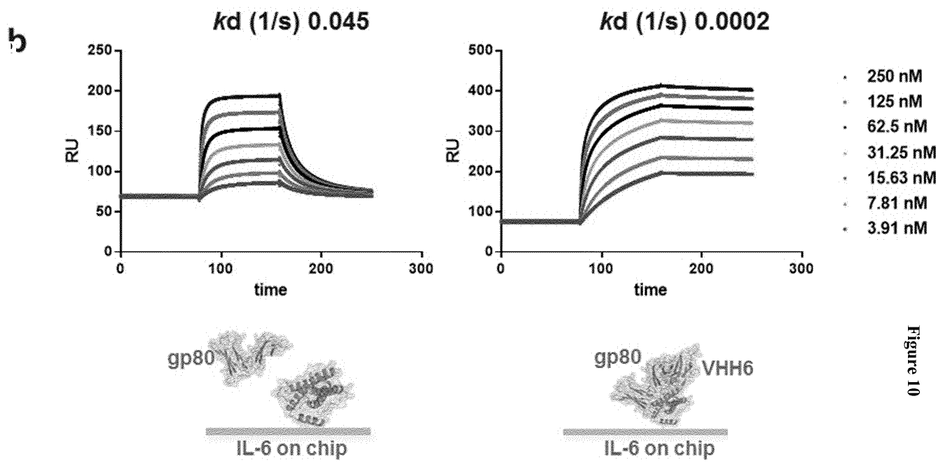

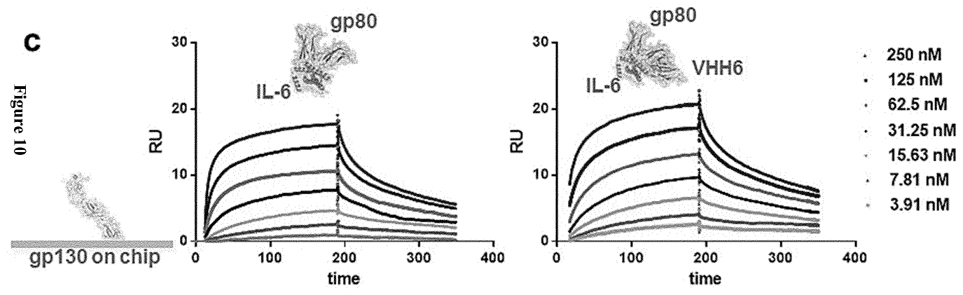

In the absence of the binding protein, the protein components of the transient PPI may have binding to one another when assessed by surface plasmon resonance at 25° C. which follows a similar pattern to that shown for IL-6 and gp80 in the absence of VHH6 in FIG. 10. Here, the sensogram curve shows a rapid dissociation phase for the gp80 with the addition of VHH6 inhibiting the dissociation curve. Other methods which can be used to detect protein:protein interactions are described below.

The two protein components of the PPI may have an equilibrium dissociation constant (K_{D}) of greater than 1 nM, or preferably greater than 10 nM in the absence of the binding protein. More preferably, this could mean a K_{D} of greater than 1 μM or most preferably even greater than 1 mM. These binding kinetics are typically determined by surface plasmon resonance at 25° C. As mentioned above, the equilibrium dissociation constant may be weak enough that binding of the two components is undetectable in the absence of the binding protein.

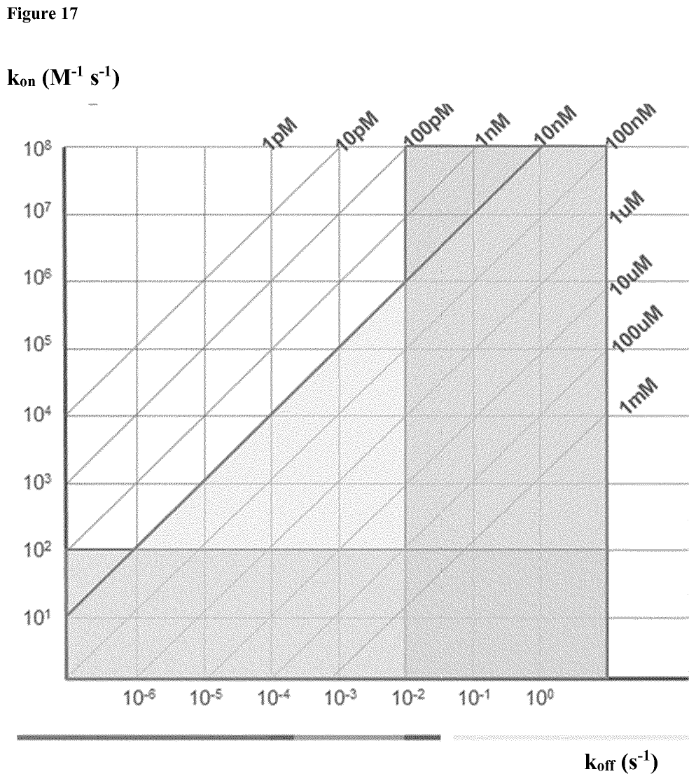

FIG. 17 illustrates the relationship between kₒₙ, k_{off} and K_{D} values. The bold diagonal line represents a K_{D} of 10 nM. Anything to the right of the line has a higher (i.e. weaker) dissociation constant than 10 nM. Anything to the left of the line has a lower (i.e. stronger/improved) dissociation constant than 10 nM. The diagonal line demonstrates how a high off rate (a high k_{off} value, e.g 10° s⁻¹) and high on rate (a high kₒₙ value e.g. 10⁸ M⁻¹s⁻¹), or a low off rate (a low k_{off} value e.g. 10⁻⁶ s⁻¹) and a low on rate (a low kₒₙ value e.g. 10² M⁻¹s⁻¹) can both result in a dissociation constant of 10 nM.

In the invention, the transient PPI may have any appropriate kₒₙ and k_{off} value that results in a K_{D} of greater than 1 nM, preferably greater than 10 nM (i.e. kₒₙ, k_{off} values which result in the K_{D} falling to the right of the 1 nM or 10 nM lines in FIG. 17 respectively). It is though typical that the weak equilibrium dissociation constant is characterised by a high off rate.

The two protein components of the transient PPI preferably have an association rate (kₒₙ) in the absence of the binding protein of less than 10² M⁻¹s⁻¹ or even less than 10 M⁻¹s⁻¹. However, as discussed above, the kₒₙ may be higher where the PPI has a fast dissociation rate. For example, with a K_{D} of greater than 10 nM the PPI could potentially have an association rate of less than 10⁸ M⁻¹ s⁻¹, optionally less than 10⁶ M⁻¹ s⁻¹, less than 10⁴ M⁻¹ s⁻¹ or less than 10³ M⁻¹ s⁻¹.

The two protein components of the transient PPI typically have a dissociation rate (k_{off}) in the absence of the binding protein of greater than 0.0001 s⁻¹ (10⁻⁴ s⁻¹), preferably greater than 0.001 s⁻¹ (10⁻³ s⁻¹), more preferably greater than 0.01 s⁻¹ (10⁻² s⁻¹) and most preferably greater than 0.025 s⁻¹. These dissociation rates may be combined with a slow association rate, such as those mentioned above, but a higher association rate with a fast dissociation rate would still make the PPI useful for the methods described below. Similarly, as discussed above, a lower dissociation rate (e.g. 10⁻⁷ s⁻¹, 10⁻⁶ s⁻¹ or 10⁻⁵ s⁻¹) could also be combined with a low association rate and still result in an overall weak affinity.

For example, for an equilibrium dissociation constant of greater than 10 nM:

The PPI may have a dissociation rate of greater than 10⁻⁷ s⁻¹, typically a dissociation rate of greater than 10⁻⁴ s⁻¹, preferably a dissociation rate of greater than 10⁻³ s⁻¹, more preferably a dissociation rate of greater than 10⁻² s⁻¹ and even more preferably a dissociation rate of greater than 10⁻¹ s⁻¹.

In the invention, the binding protein specific for the junctional epitope created by the transient PPI stabilises the PPI. In terms of kₒₙ for the interaction of the two proteins of the PPI, this is typically greater than 10² M⁻¹ s⁻¹, optionally greater than 10³ M⁻¹ s⁻¹ in the presence of the binding protein. The kₒₙ could also be greater than 10⁴ M⁻¹ s⁻¹ or 10⁵ M⁻¹ s. The k_{off} for the interaction of the two proteins of the PPI is typically less than 0.01 s⁻¹, optionally less than 0.001 s⁻¹ or preferably less than 0.0005 s⁻¹ in the presence of the binding protein. The dissociation constant (K_{D}) for the interaction of the two proteins of the PPI is typically less than 10 nM, preferably less than 1 nM or more preferably less than 100 pM in the presence of the binding protein. Once again, these binding kinetics are typically determined by surface plasmon resonance at 25° C.

Use of a binding protein and PPI in the methods described below is largely dependent on how much the binding protein stabilises the interaction, rather than necessarily on specific values for the k_{off}, kₒₙ and K_{D}. The K_{D} for the protein:protein interaction may reduced (i.e. improved) at least 10 fold, at least 50 fold or even at least 100 fold in the presence of the binding protein. Even a 10 fold stabilisation of the interaction would greatly increase the therapeutic utility of a complex and thus allow for its use in one of the methods described below. Preferably, in the present invention the transient PPI is characterised by a k_{off} in the absence of the binding protein of greater than 0.01 s⁻¹, greater than 0.025 s⁻¹ or even greater than 0.04 s⁻¹. In the presence of the binding protein, the k_{off} is preferably reduced to less than 0.01 s⁻¹, optionally less than 0.001 s⁻¹ or preferably less than 0.0005 s⁻¹. Stabilisation of the PPI by the binding protein may reduce the k_{off} at least 50 fold, 100 fold or 200 fold. A decrease in k_{off} of at least 10 fold could be sufficient for stabilisation of the PPI to find use in the methods described below.

Preferably, both the k_{off} and the K_{D} are reduced (improved) at least 10 fold, more preferably at least 50 fold and most preferably at least 100 fold in the presence of the binding protein.

In the invention, the transient PPI is preferably not between two domains of an antibody (i.e. between the V_{H} and V_{L} domains of an antibody).

In the present invention, the transient PPI is typically the IL-6/gp 80 interaction. Other examples of suitable transient PPIs potentially include antibody:antigen interactions.

As mentioned above, a protein:protein interaction could also be engineered so that the kinetics of the interaction make it appropriate for stabilisation via a binding protein of the invention and for use in the methods described below. For example, residues at the binding interface for a high affinity/low dissociation rate interaction could be mutated to reduce the affinity and/or increase the dissociation rate of the complex. The interaction may then be undetectable in the absence of the binding protein using techniques such as ELISA, gel filtration (size exclusion chromatography) or surface plasmon resonance.

In the present invention, the binding protein is specific (or selective) for a junctional epitope created by the transient PPI. The binding protein specifically binds at or over the junction (interface) created when the two protein components of a transient PPI form a complex. This means that the binding protein is not allosteric, i.e. the binding protein does not recognise a site created when the two protein components of the PPI come together that is located away from the protein:protein interface.

“Selectively binds” or “selectively recognises” a transient PPI means the binding protein binds with preferential or high affinity to the PPI complex for which it is selective but does not substantially bind, or binds with very low affinity, to other proteins (e.g. the individual components of the PPI). Typically, as discussed further below, the binding protein has no detectable binding to each individual component of the protein:protein interaction.

By specific (or selective), it will be understood that the binding protein binds to the PPI of interest with no significant cross-reactivity to any other molecule, which may include the individual protein components of the PPI. Cross-reactivity may be assessed by any suitable method described herein. Cross-reactivity of a binding protein (such as an antibody) with a molecule other than the PPI may be considered significant if the binding protein binds to the other molecule at least about 10%, at least 20%, at least 30%, at least 40% or at least 50% as strongly as it binds to the PPI complex of interest. A binding protein that is specific (or selective) for the PPI may bind to another molecule at less than about 50%, less than about 40%, less than about 30%, less than about 20% or less than 10% the strength that it binds to the PPI complex. The binding protein may bind to the other molecule at less than about 20%, less than about 15%, less than about 10% or less than about 5%, less than about 2% or less than about 1% the strength that it binds to the PPI. Binding of less than 10%, less than 5% or less than 1% is preferable for binding to the individual protein components of the PPI when compared with binding to the PPI complex.

The rates at which a binding protein binds to a PPI (or other molecule) is referred to herein as the “on” rate” k_{on-bp} and the rate at which the binding protein dissociates is referred to herein as the “off” rate or k_{off-bp}. As used herein, the symbol “K_{D-bp}” denotes the binding dissociation constant of a binding protein for a PPI complex (or other molecule). K_{D-bp} is defined as k_{off-bp}/k_{on-bp}.

The K_{D-bp} value of the binding protein for binding to a PPI complex of interest may be at least about 10 times, 100 times, 200 times, 300 times or 500 times lower (better) than the K_{D-bp} value for binding to the individual protein components of the PPI. Such low binding would not interfere with use of the binding protein in the methods described below.

The binding affinity may be given in any appropriate units, such as μM, nM or pM. The smaller the K_{D-bp} value, the larger the binding affinity of the binding protein to the PPI complex.

The decrease in the K_{D-bp} value of the binding protein for binding to the PPI complex compared to the K_{D-bp} value of the binding protein binding to the individual components may result from an increase in the on rate (k_{on-bp}) and/or a decrease in the off rate (k_{off-bp}).

The on rate (k_{on-bp}) of the binding protein binding to the PPI complex is generally increased compared to the on rate of the binding protein binding to the individual components of the PPI. The off rate (k_{off-bp}) of the binding protein binding to the PPI complex is generally decreased compared to the off rate of the binding protein binding to the individual components of the PPI. Most typically, the on rate is increased and the off-rate is decreased.

The k_{on-bp}, k_{off-bp}, and K_{D-bp} values may be determined using any appropriate technique, but are typically determined using surface plasmon resonance at 25° C.

The K_{D-bp} value of the binding protein binding to a PPI complex may be less than 1 nM, less than 500 pM, less than 100 pM or less than 10 pM

Most preferably, the binding protein shows undetectable binding to the individual protein components of the protein:protein interaction. Methods of identifying whether a binding protein binds a complex formed by a protein:protein interaction, but not to each individual component of the protein:protein interaction are described in the Examples. Methods include ELISA, where the individual protein components and a complex of the PPI may be immobilised overnight on plates before addition of the binding protein. Other techniques which can be used to determine whether a binding protein binds a complex formed by a protein:protein interaction, but not to each individual component of the protein:protein, are size exclusion chromatography (HPLC), MicroScale Thermophoresis or Octet (Bio-Layer Interferometry). As discussed above, surface plasmon resonance may also be used. Once again, these techniques would be readily known to the skilled persona and are described in the Examples. In the present invention, the binding protein may show undetectable binding to each individual component of the PPI using any one, preferably multiple, more preferably all, of the above techniques.

The binding protein binds an epitope which spans across the interface between the two protein components of the PPI complex. Between 20% and 80% of the epitope is typically present on one of the proteins of the protein:protein interaction, with the remainder of the epitope present on the other protein. Preferably, between 30% and 70% of the epitope is present on one of the proteins of the protein:protein interaction, with the remainder of the epitope present on the other protein. More preferably, between 40% and 60% of the epitope is present on one of the proteins of the protein:protein interaction, with the remainder of the epitope present on the other protein. In the present context, “epitope” refers to the amino acid residues recognised by the binding protein. In other words, between 20% and 80% of the amino acids recognised by the binding protein are typically present on one of the proteins of the protein:protein interaction, with the remainder of the amino acids recognised by the binding protein present on the other protein. Preferably, between 30% and 70% of the amino acids recognised by the binding protein are present on one of the proteins of the protein:protein interaction, with the remainder of the amino acids recognised by the binding protein present on the other protein. More preferably, between 40% and 60% of the amino acids recognised by the binding protein are present on one of the proteins of the protein:protein interaction, with the remainder of the amino acids recognised by the binding protein present on the other protein. Methods of determining a binding epitope are described in the Examples below and include crystallography and hydrogen-deuterium exchange mass spectrometry. For example, an epitope residue may be classed as a residue within 4 Å of an antibody paratope residue.

### Junctional Antibody

The binding protein is preferably an antibody or an antibody fragment (a junctional antibody). An antibody refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as HCVR or V_{H}) and a heavy chain constant region. Each light chain is comprised of a light chain variable region (abbreviated herein as LCVR or V_{L}) and a light chain constant region. The variable regions of the heavy and light chains contain a binding domain that interacts with an antigen. The V_{H} and V_{L} regions can be further subdivided into regions of hypervariability, termed complementarity determining regions (CDR), interspersed with regions that are more conserved, termed framework regions (FR).

The constant regions of the antibodies may mediate the binding of the immunoglobulin to host tissues or factors, including various cells of the immune system (e.g., effector cells) and the first component (C1q) of the classical complement system.

An antibody of the invention will typically be a monoclonal antibody.

Antibodies generated against PPI complexes may be obtained, where immunisation of an animal is necessary, by administering the polypeptides to an animal (see below), e.g. a non-human animal, using well-known and routine protocols, see for example Handbook of Experimental Immunology, D. M. Weir (ed.), Vol 4, Blackwell Scientific Publishers, Oxford, England, 1986). Many warm-blooded animals, such as rabbits, mice, rats, sheep, cows, camels, llamas or pigs may be immunized. However, mice, rabbits, pigs and rats are typically suitable, as are camels or llamas for the generation of VHH antibodies.

Monoclonal antibodies may be prepared by the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Antibodies may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15): 7843-78481; WO92/02551; WO2004/051268 and WO2004/106377.

Antibodies can also be generated using various phage display methods known in the art and include those disclosed by Brinkman et al. (in J. Immunol. Methods, 1995, 182: 41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

Fully human antibodies are those antibodies in which the variable regions and the constant regions (where present) of both the heavy and the light chains are all of human origin, or substantially identical to sequences of human origin, but not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP 0546073, U.S. Pat. Nos. 5,545,806, 5,569,825, 5,625,126, 5,633,425, 5,661,016, 5,770,429, EP 0438474 and EP 0463151.

The antibody molecules of the present invention may comprise a complete antibody molecule having full length heavy and light chains or a fragment or antigen-binding portion thereof. The term “antigen-binding portion” of an antibody refers to one or more fragments of an antibody that retain the ability to selectively bind to an antigen. It has been shown that the antigen-binding function of an antibody can be performed by fragments of a full-length antibody. Antibodies and fragments and antigen binding portions thereof include Fab, modified Fab, Fab′, modified Fab′, F(ab′)₂, Fv, single domain antibodies (e.g. V_{H} or V_{L} or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments include the Fab and Fab′ fragments described in International patent applications WO 2005/003169, WO 2005/003170 and WO 2005/003171 and Fab-dAb fragments described in International patent application WO2009/040562. These antibody fragments may be obtained using conventional techniques known to those of skill in the art, and the fragments may be screened for utility in the same manner as intact antibodies.

In the invention, the antibody is preferably a Fab, scFV or VHH antibody. These types of antibody have short serum half lives and rapid renal clearance, so are useful in the therapeutic methods described below. Other suitable antibody formats include fynomers, human nanobodies and shark IgNARs.

The constant region domains of the antibody molecule of the present invention, if present, may be selected having regard to the proposed function of the antibody molecule, and in particular the effector functions which may be required. For example, the constant region domains may be human IgA, IgD, IgE, IgG or IgM domains. In particular, human IgG constant region domains may be used, especially of the IgG1 and IgG3 isotypes when the antibody molecule is intended for therapeutic uses and antibody effector functions are required. Alternatively, IgG2 and IgG4 isotypes may be used when the antibody molecule is intended for therapeutic purposes and antibody effector functions are not required.

An antibody of the invention may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences.

An antibody of the invention may be a human antibody or a humanised antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

Such a human antibody may be a human monoclonal antibody. Such a human monoclonal antibody may be produced by a hybridoma that includes a B cell obtained from a transgenic nonhuman animal, e.g., a transgenic mouse, having a genome comprising a human heavy chain transgene and a light chain transgene fused to an immortalized cell.

Human antibodies may be prepared by in vitro immunisation of human lymphocytes followed by transformation of the lymphocytes with Epstein-Barr virus.

The term “human antibody derivatives” refers to any modified form of the human antibody, e.g., a conjugate of the antibody and another agent or antibody.

The term “humanized antibody” is intended to refer to CDR-grafted antibody molecules in which CDR sequences derived from the germline of another mammalian species, such as a mouse, have been grafted onto human framework sequences. Additional framework region modifications may be made within the human framework sequences.

As used herein, the term ‘CDR-grafted antibody molecule’ refers to an antibody molecule wherein the heavy and/or light chain contains one or more CDRs (including, if desired, one or more modified CDRs) from a donor antibody (e.g. a murine or rat monoclonal antibody) grafted into a heavy and/or light chain variable region framework of an acceptor antibody (e.g. a human antibody). For a review, see Vaughan et al, Nature Biotechnology, 16, 535-539, 1998. In one embodiment rather than the entire CDR being transferred, only one or more of the specificity determining residues from any one of the CDRs described herein above are transferred to the human antibody framework (see for example, Kashmiri et al., 2005, Methods, 36, 25-34). In some circumstances only the specificity determining residues from one or more of the CDRs described herein above are transferred to the human antibody framework. In other circumstances only the specificity determining residues from each of the CDRs described herein above are transferred to the human antibody framework.

When the CDRs or specificity determining residues are grafted, any appropriate acceptor variable region framework sequence may be used having regard to the class/type of the donor antibody from which the CDRs are derived, including mouse, primate and human framework regions. Suitably, the CDR-grafted antibody has a variable domain comprising human acceptor framework regions as well as one or more of the CDRs or specificity determining residues described above. Thus, provided in one aspect is a neutralising CDR-grafted antibody wherein the variable domain comprises human acceptor framework regions and non-human donor CDRs.

Examples of human frameworks which can be used are KOL, NEWM, REI, EU, TUR, TEI, LAY and POM (Kabat et al., supra). For example, KOL and NEWM can be used for the heavy chain, REI can be used for the light chain and EU, LAY and POM can be used for both the heavy chain and the light chain. Alternatively, human germline sequences may be used; these are available for example at: http://www.vbase2.org/ (see Retter et al, Nucl. Acids Res. (2005) 33 (supplement 1), D671-D674).

In a CDR-grafted antibody, the acceptor heavy and light chains do not necessarily need to be derived from the same antibody and may, if desired, comprise composite chains having framework regions derived from different chains.

Also, in a CDR-grafted antibody, the framework regions need not have exactly the same sequence as those of the acceptor antibody. For instance, unusual residues may be changed to more frequently occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in WO 91/09967.

It will also be understood by one skilled in the art that antibodies may undergo a variety of posttranslational modifications. The type and extent of these modifications often depends on the host cell line used to express the antibody as well as the culture conditions. Such modifications may include variations in glycosylation, methionine oxidation, diketopiperazine formation, aspartate isomerization and asparagine deamidation. A frequent modification is the loss of a carboxy-terminal basic residue (such as lysine or arginine) due to the action of carboxypeptidases (as described in Harris, R J. Journal of Chromatography 705:129-134, 1995).

The antibody heavy chain may comprise a CH1 domain and the antibody light chain comprises a CL domain, either kappa or lambda.

Biological molecules, such as antibodies or fragments, contain acidic and/or basic functional groups, thereby giving the molecule a net positive or negative charge. The amount of overall “observed” charge will depend on the absolute amino acid sequence of the entity, the local environment of the charged groups in the 3D structure and the environmental conditions of the molecule. The isoelectric point (pI) is the pH at which a particular molecule or surface carries no net electrical charge. In one embodiment the antibody or fragment according to the present disclosure has an isoelectric point (pI) of at least 7. In one embodiment the antibody or fragment has an isoelectric point of at least 8, such as 8.5, 8.6, 8.7, 8.8 or 9. In one embodiment the pI of the antibody is 8. Programs such as ** ExPASY http://www.expasy.ch/tools/pi_tool.html (see Walker, The Proteomics Protocols Handbook, Humana Press (2005), 571-607) may be used to predict the isoelectric point of the antibody or fragment.

The binding protein may not be a bispecific antibody, where one arm of the bispecific antibody contacts one protein component of the PPI and the second arm of the bispecific antibody contacts the second protein component of the PPI.

Typically, a single heavy chain variable region of the antibody or fragment thereof has interactions with both proteins of the transient PPI, a single light chain variable region has interactions with both proteins of the transient PPI or both single heavy and light chain variable regions have interactions with both proteins of the transient PPI. In other words, a single heavy chain variable region of the antibody or fragment thereof contacts both proteins of the transient PPI, a single light chain variable region contacts both proteins of the transient PPI or both single heavy and light chain variable regions contact both proteins of the transient PPI. This can be determined by X-ray crystallography, where contact may be defined as residues within 4 Å of an antibody paratope residue. Mutational analysis and analysing changes in binding kinetics may additionally be used.

Preferably, one or more individual CDRs of the antibody interact with both proteins of the PPI. For example, one of the CDRs of the heavy chain, possibly two CDRs of the heavy chain, or even all three CDRs of the heavy chain may interact with both proteins of the PPI. Likewise, one of the CDRs of the light chain, possibly two CDRs of the light chain, or even all three CDRs of the light chain may interact with both proteins of the PPI. In other words, one or more individual CDRs of the antibody contact both proteins of the PPI. For example, one of the CDRs of the heavy chain, possibly two CDRs of the heavy chain, or even all three CDRs of the heavy chain may contact both proteins of the PPI. Likewise, one of the CDRs of the light chain, possibly two CDRs of the light chain, or even all three CDRs of the light chain may contact both proteins of the PPI. Once again, this can be determined by X-ray crystallography (where contact may be defined as residues within 4 Å of the antibody paratope residue), optionally in combination with mutational analysis and measuring changes in binding kinetics.

Where the PPI is the IL-6/gp80 interaction, the antibody or fragment may comprise at least one heavy chain CDR sequence selected from SEQ ID NOs: 2-4. These are the CDRs of the VHH6 antibody described in the Examples. The antibody may comprise a HCDR3 of SEQ ID NO: 4. The antibody may additionally comprise a HCDR1 of SEQ ID NO: 2 and/or a HCDR2 of SEQ ID NO: 3.

The antibody may also comprise the CDR sequences present in SEQ ID NO: 1 (the V_{H} of VHH6) as determined by the Kabat or Chothia methods. These methods are well known in the art and would be readily understood by the skilled person.

The antibody of the invention may also comprise a V_{H} of SEQ ID NO: 1

Alternatively the antibody of the invention is a humanized VHH6 antibody. In a specific embodiment, said antibody comprises at least at least one heavy chain CDR sequence selected from SEQ ID NOs: 147 and 148. The antibody may comprise a HCDR3 of SEQ ID NO: 152 or 153. The antibody may additionally comprise a HCDR1 of SEQ ID NO: 149 or 150 and/or a HCDR2 of SEQ ID NO: 151. More specifically said antibody comprises HCDR1 of SEQ ID NO: 149, HCDR2 of SEQ ID NO: 151 and HCDR3 of SEQ ID NO: 152. Alternatively such antibody comprises HCDR1 of SEQ ID NO: 150, HCDR2 of SEQ ID NO: 151 and HCDR3 of SEQ ID NO: 153. The antibody may alternatively be or may comprise a variant of one of the specific sequences recited above. For example, a variant may be a substitution, deletion or addition variant of any of the above amino acid sequences.

A variant antibody may comprise 1, 2, 3, 4, 5, up to 10, up to 20 or more amino acid substitutions and/or deletions from the specific sequences discussed above. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants typically involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, charged (+) | Thr | polar, hydrophilic, neutral |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

“Derivatives” or “variants” generally include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

A variant antibody may have a V_{H} sequence at least 90% (preferably 95%) identical to SEQ ID NO: 1. A variant may have at least 90% or 95% identity to SEQ ID NO: 1, but retaining the exact CDR sequences of SEQ ID NOs: 2, 3 and 4.

A variant antibody may have a V_{H} sequence at least 90% (preferably 95%) identical to SEQ ID NO: 147 or 148. A variant may have at least 90% or 95% identity to SEQ ID NO: 147 or 148, but retaining the corresponding exact CDR sequences of SEQ ID NOs: 149-153.

This level of amino acid identity may be seen across the full length of the relevant SEQ ID NO sequence or over a part of the sequence, such as across about 20, 30, 50, 75, 100, 150, 200 or more amino acids, depending on the size of the full length polypeptide. Typically, identity is measured over the full length of the relevant sequence.

In connection with amino acid sequences, “sequence identity” refers to sequences which have the stated value when assessed using ClustalW (Thompson et al., 1994, supra) with the following parameters:

Pairwise alignment parameters—Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatized.

Antibodies having specific sequences and variants which maintain the function or activity of these chains are therefore provided.

A polynucleotide sequence of SEQ ID NO: 5 encodes the V_{H} of SEQ ID NO: 1.

The present invention also provides an isolated DNA sequence encoding the heavy and/or light chain(s) of any antibody molecule of the present invention. Suitably, the DNA sequence encodes the heavy or the light chain of an antibody molecule of the present invention.

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing. Generally, a variant has 1-20, 1-50, 1-75 or 1-100 substitutions and/or deletions.

Suitable variants may be at least about 70% homologous to a polynucleotide of any one of nucleic acid sequences disclosed herein, typically at least about 80 or 90% and more suitably at least about 95%, 97% or 99% homologous thereto. Variants may retain at least about 90%, 91%, 92%, 93%, 94%, 95%, 96%, 97%, 98% or 99% identity. Variants typically retain about 60%-about 99% identity, about 80%-about 99% identity, about 90%-about 99% identity or about 95%-about 99% identity. Homology and identity at these levels is generally present at least with respect to the coding regions of the polynucleotides. Methods of measuring homology are well known in the art and it will be understood by those of skill in the art that in the present context, homology is calculated on the basis of nucleic acid identity. Such homology may exist over a region of at least about 15, at least about 30, for instance at least about 40, 60, 100, 200 or more contiguous nucleotides (depending on the length). Such homology may exist over the entire length of the unmodified polynucleotide sequence.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p 387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, typically less than about 0.1, suitablyless than about 0.01, and most suitably less than about 0.001. For example, the smallest sum probability may be in the range of about 1-about 0.001, often about 0.01-about 0.001.

The homologue may differ from a sequence in the relevant polynucleotide by less than about 3, 5, 10, 15, 20 or more mutations (each of which may be a substitution, deletion or insertion). For example, the homologue may differ by 3-50 mutations, often 3-20 mutations. These mutations may be measured over a region of at least 30, for instance at least about 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

The DNA sequence of the present invention may comprise synthetic DNA, for instance produced by chemical processing, cDNA, genomic DNA or any combination thereof.

DNA sequences which encode an antibody molecule of the present invention can be obtained by methods well known to those skilled in the art. For example, DNA sequences coding for part or all of the antibody heavy and light chains may be synthesised as desired from the determined DNA sequences or on the basis of the corresponding amino acid sequences.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

Also provided is a host cell comprising one or more cloning or expression vectors comprising one or more DNA sequences encoding an antibody of the present invention. Any suitable host cell/vector system may be used for expression of the DNA sequences encoding the antibody molecule of the present invention. Bacterial, for example E. coli, and other microbial systems may be used or eukaryotic, for example mammalian, host cell expression systems may also be used. Suitable mammalian host cells include CHO, myeloma or hybridoma cells.

The present invention also provides a process for the production of an antibody molecule according to the present invention comprising culturing a host cell containing a vector of the present invention under conditions suitable for leading to expression of protein from DNA encoding the antibody molecule of the present invention, and isolating (collecting) the antibody molecule.

Antibodies may bind to the same epitope, or compete for binding with the VHH6 antibody of the invention. Residues in the VHH6 epitope are identified in the Examples and Figures. In particular, the VHH6 epitope comprises residues D221, 5228, 8231 and H256 of gp80, and residues S22, E23, E80 and Q183 of IL-6. An antibody of the invention may bind an epitope which comprises at least one of these residues from gp80 and at least one of the residues from IL-6, preferably at least two or at least three residues from both IL-6 and gp80. Most preferably, an antibody of the invention binds an epitope with all of the above residues. Antibody epitopes may be determined by x-ray crystallography analysis. Antibodies of the present invention may therefore be assessed through x-ray crystallogray analysis of the antibody bound to the IL-6/gp80 complex. Epitopes may, in particular, be identified in this way by determining residues on IL-6/gp80 within 4 Å of an antibody paratope residue.

One can easily determine whether an antibody binds to the same epitope as, or competes for binding with, a reference antibody by using other routine methods known in the art. For example, to determine if a test antibody binds to the same epitope as a reference antibody of the invention, the reference antibody is allowed to bind to a protein or peptide under saturating conditions. Next, the ability of a test antibody to bind to the protein or peptide is assessed. If the test antibody is able to bind to the protein or peptide following saturation binding with the reference antibody, it can be concluded that the test antibody binds to a different epitope than the reference antibody. On the other hand, if the test antibody is not able to bind to protein or peptide following saturation binding with the reference antibody, then the test antibody may bind to the same epitope as the epitope bound by the reference antibody of the invention.

To determine if an antibody competes for binding with a reference antibody, the above-described binding methodology is performed in two orientations. In a first orientation, the reference antibody is allowed to bind to a protein/peptide under saturating conditions followed by assessment of binding of the test antibody to the protein/peptide molecule. In a second orientation, the test antibody is allowed to bind to the protein/peptide under saturating conditions followed by assessment of binding of the reference antibody to the protein/peptide. If, in both orientations, only the first (saturating) antibody is capable of binding to the protein/peptide, then it is concluded that the test antibody and the reference antibody compete for binding to the protein/peptide. As will be appreciated by the skilled person, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50%, 75%, 90% or even 99% as measured in a competitive binding assay (see, e.g., Junghans et al., Cancer Res, 1990:50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

To screen for antibodies that bind to a particular epitope, a routine cross-blocking assay such as that described in Antibodies, Harlow and Lane (Cold Spring Harbor Press, Cold Spring Harb., N.Y.) can be performed. Other methods include alanine scanning mutants, peptide blots (Reineke (2004) Methods Mol Biol 248:443-63), or peptide cleavage analysis. In addition, methods such as epitope excision, epitope extraction and chemical modification of antigens can be employed (Tomer (2000) Protein Science 9: 487-496). Such methods are well known in the art.

Binding to the same epitope or competitive binding could also be determined using X-ray crystallography as mentioned above.

A binding protein of the invention does not have to be an antibody. A binding protein could be any type of protein which can be selected for binding to the transient protein:protein interaction. For example, the binding protein may comprise ankyrin repeat domains.

### Use of a Binding Protein Specific for a Junctional Epitope Created by a Transient Protein:Protein Interaction in Delivering Cargo to a Target Cell

The invention provides use of a binding protein as described above in delivering cargo to a target cell. The cargo can be a diagnostic or therapeutic compound. The target cell can be any appropriate cell type, but is typically a cancer cell.

Examples of cancer cells include cells of a haematologic tumour, such as cells selected from the group consisting of acute myeloic leukemia cells, chronic myeloic leukemia cells, acute lymphatic leukemia cells, chronic lymphatic leukemia cells, lymphoma cells, myeloproliferative syndrome cells, myelodysplasia cells, myeloma cells, cells of a non-haematologic tumour, such as a cell selected from the group consisting of renal cell carcinoma cells, bladder cancer cells, lung cancer cells, mesothelioma cells, prostate cancer cells, brain cancer cells, bone cancer cells, sarcoma cells, soft tissue cancer cells, ovarian cancer cells, cervix cancer cells, breast cancer cells, endometrial cancer cells, uterine cancer cells, germ cell tumour cells, anal cancer cells, rectal carcinoma cells, colon carcinoma cells, small intestine carcinoma cells, gastric carcinoma cells, gastrointestinal stroma tumour cells, liver carcinoma cells, pancreas carcinoma cells, bile duct carcinoma cells, gall bladder carcinoma cells, head and neck cancer cells, hypopharyngeal cancer cells, laryngeal cancer cells, cells of a cancer of the esophagus, skin cancer cells, preferably melanoma cells, cells of a childhood cancer, cells of an endocrine tumour, cells of a carcinoid tumour, thymoma cells, thyroid cancer cells, cells of an islet cell tumour, cells of an adrenal cell tumour, cells of a neuroendocrine tumour and cells of a cancer of unknown primary (cancer of unknown primary origin).

As discussed above, the present invention provides a more specific means for targeting cargo to a cell. This method involves recognition of two different target molecules on the target cell and then delivery of the cargo via the binding protein of the invention. The target molecules may for example be antigens or receptors, such as tumour antigens on a tumour cell. The term “antigen” generally encompasses molecules capable of being recognised by an antibody. The term “antigen” may encompass receptors. However, receptors are also specifically discussed herein as the targeting portion of the targeting molecule may be a ligand for a receptor (rather than an antibody).

The combination of both antigens (or receptors) may only be found on the target (e.g. tumour cells) or expressed on cells that are not target cells (i.e. non-cancerous cells) to a negligible extent. Both tumour antigens may be specific for cancerous cells, and for a certain type of cancer.

Antigen pairings are provided for example in WO 2013/104804, such as:
(i) one of said antigens is EpCAM and the other one is EGFR, HER2/neu, CD 10, VEGF-R or MDR;
(ii) one of said antigens is MCSP and the other one is melanoferrin or EpCAM;
(iii) one of said antigens is CA125 and the other one CD227
(iv) one of said antigens is CD56 and the other one is CD 140b or GD3 ganglioside;
(v) one of said antigens is EGFR and the other one is HER2;
(vi) one of said antigens is PSMA and the other one is HER2;
(vii) one of said antigens is Sialyl Lewis and the other one is EGFR;
(viii) one of said antigens is CD44 and the other one is ESA, CD24, CD133, MDR or CD117;
(ix) one of said antigens is CD34 and the other one is CD19, CD79a, CD2, CD7, HLA-DR, CD13, CD117, CD33 or CD15;
(x) one of said antigens is CD33 and the other one is CD 19, CD79a, CD2, CD7, HLA-DR, CD13, CD117 or CD15;
(xi) one of said antigens is MUC1 and the other one is CD10, CEA or CD57;
(xii) one of said antigens is CD38 and the other one is CD 138;
(xiii) one of said antigens is CD 24 and the other one is CD29 or CD49f;
(xiv) one of said antigens is carbonic anhydrase IX and the other one is aquaporin-2;
(xv) one of said antigens is HLA-A2 and the other one is EpCAM;
(xvi) one of said antigens is HLA-A2 and the other one is CD45;
(xvii) one of said antigens is HLA-A2 and the other one is EGFR;
(xviii) one of said antigens is HLA-A2 and the other one is Her2;
(xix) one of said antigens is HLA-A2 and the other one is CEA;
(xx) one of said antigens is EpCAM and the other one is CEA;
(xxi) one of said antigens is CD45 and the other one is CD 138;
(xxii) one of said antigens is EGFR and the other one is CEA;
(xxiii) one of said antigens is Her2 and the other one is CEA; or
(xxiv) one of said antigens is CD19 and the other one is a clonotypic antibody on the surface of a B cell.

Other antigens expressed on different cell types are well known in the art and could also be identified using routine experimentation. Examples of markers for the malignant state of a cell include: E-cadherin for epithelial cells and ductal-type breast carcinoma cells; Ca-125 for Epitheloid malignancies and ovary cancer cells, adenocarcinoma cells and breast cancer cells; Her-2/neu for breast cancer cells; gross cystic disease fluid protein (BRST-2 protein) for breast cancer cells; BCA-225 (breast carcinoma associated glycoprotein) for lung and breast cancer cells; CA 19-9 (carbohydrate antigen 19-9) for pancreas, bile duct and intestinal tract cancer cells; CEA for colorectal cancer cells; CD117 (c-kit) for gist (gastrointestinal stromal tumour) cells (and myeloid and mast cells); CD30 for Reed-Sternberg cells (and i-1 activated T-cells and B-cells); Epithelial antigen (BER-EP4), Epithelial membrane antigen, and Epithelial Related Antigen (MOC-31) for epithelial cancer cells; Epidermal growth factor receptor (HER1) for cells of various cancers; Platelet derived growth factor receptor (PDGFR) alpha for cells of various cancers; Melanoma associated marker/Mart 1/Melan-A for melanoma cells; CD133 for cancer stem cell populations and others; TAG 72 (tumour associated gp 72) for adenocarcinoma cells.

Further examples for markers for a malignant state of a cell/cells include: EpCAM, CD 19, HER-2, HER-3, HER-4, PSMA, MUC-1 (mucin), MUC2, MUC3, MUC4, MUC5AC, MUC5B, MUC7, Lewis-Y, CD20, CD33, CD44v6, Wue-1, Plasma Cell Antigen, (membrane-bound) IgE, Melanoma Chondroitin Sulfate Proteoglycan (MCSP), STEAP, mesothelin, Prostate Stem Cell Antigen (PSCA), sTn (sialylated Tn antigen), FAP (fibroblast activation antigen), EGFRvIII, Iga, Ig, MT-MMPs, Cora antigen, EphA2, L6 and CO-29, CCR5, pHCG, ganglioside GD3, 9-0-Acetyl-GD3, GM2, Globo H, fucosyl GM1, Poly SA, GD2, Carboanhydrase IX (MN/CA IX), Sonic Hedgehog (Shh), CCR8, TNF-alpha precursor, A33 Antigen, Ly-6, desmoglein 4, E-cadherin neoepitope, Fetal Acetylcholine Receptor, CD25, Muellerian inhibitor Substance (MIS) Receptor type II, endosialin, SAS, CD63, TF-antigen, CD7, CD22, Iga(CD79a), Ig (CD79b), G250, gp100, F19-antigen and EphA2.

Examples for antigens that are specific for a certain cell type/cell lineage or for a few cell types/cell lineages (cell type markers/cell lineage markers) include: CD45 for hematopoietic cells; CD34 for endothelial cells, stem cells, and stromal cells; CD33 for myeloid cells; CD 138 for plasma cells and a subset of epithelial cells; CD 15 for epithelial, myeloid, and Reed-Sternberg cells; CD 1a for cortical thymocyctes and Langerhans cells; CD2 for thymic cells, T-cells, and Natural Killer (NK) cells; CD3 for T-cells; CD4 for helper T-cells; CD5 for T-cells, a subset of B-cells, and thymic carcinoma cells; CD8 for cytotoxic T-cells; CD20 for B-cells; CD23 for activated B-cells; CD31 for endothelial cells; CD43 for T-cells, myeloid cells, a subset of B-cells, histiocytes, and plasma cells; CD56 for NK cells; CD57 for neuroendocrine cells, and NK cells; CD68 for macrophages; CD79a for B-cells and plasma cells; CD 146 for the endothelial cell lineage; surfactant proteins for lung cells; synaptophysin, CD56 or CD57 for neuroendocrine cells; nicotinic acetylcholine receptor or muscle-specific kinase (MUSK) for muscle cells; voltage-gated calcium channel (P/Q-type) or voltage-gated potassium channel (VGKC) or N-methyl-D-aspartate receptor (NMD A) for muscle cells and neurons; TSH (thyroid stimulating hormone) receptor for thyreoid gland; amphiphysin for muscle cells; HepPar-1 for hepatocytes; ganglioside GQ1B, GD3 or GM1 for neuronal cells; and glycophorin-A for cells of the erythropoietic cell lineage.

Specifically, the present invention provides a method of delivering cargo to a target cell, comprising:

(1) administering two targeting molecules, wherein:
the first targeting molecule comprises a first targeting portion which binds specifically to a first target molecule on the target cell and wherein the targeting portion is linked to the first protein component of a transient protein:protein interaction; and
the second targeting molecule comprises a second targeting portion which binds specifically to a second target molecule on the target cell and wherein the targeting portion is linked to the second protein component of the transient protein:protein interaction;

(2) administering a binding protein specific for a junctional epitope created by the transient protein:protein interaction, which binding protein is linked to the cargo and wherein the binding protein is as defined above.

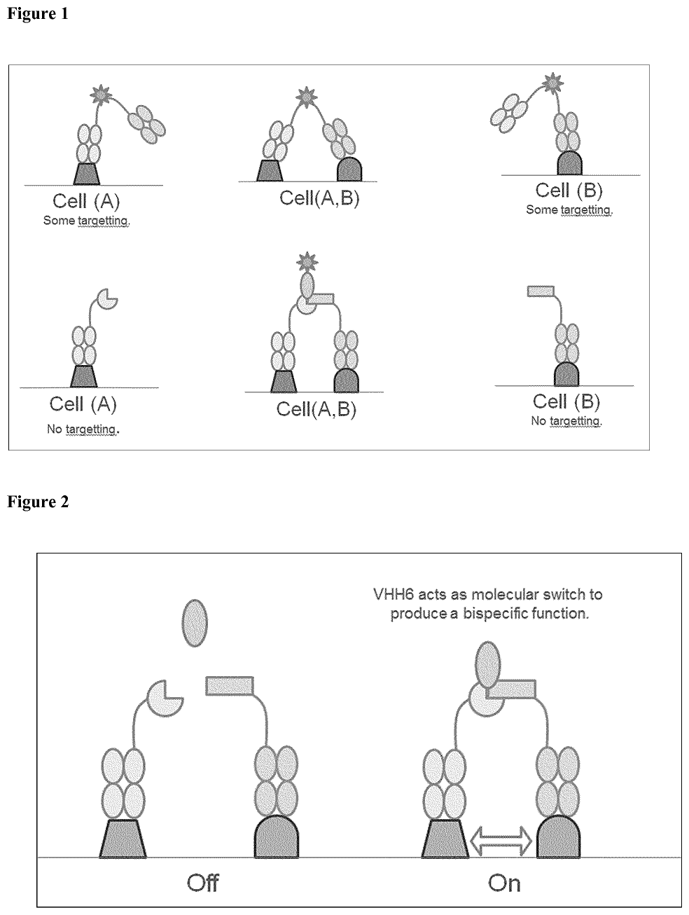

This method is exemplified in FIG. 1.

The first and second targeting portions are typically antibodies or antibody fragments (antibody fragments are described above), which specifically bind the first and second target molecules on the target cell. The targeting portions may also be ligands where the target molecules are receptors. Specific binding is discussed above. The targeting portions may linked to the protein components of the PPI by an appropriate means, such as a linker sequence. Methods of fusing proteins whilst retaining functionality are well known in the art.

The second step of the method involves administration of the binding protein, which, as discussed above, is typically an antibody or antibody fragment. The cargo may be attached to the binding protein by any appropriate means. The cargo may be a diagnostic or therapeutic agent. For example, the therapeutic compound may be a radiolabel (radioactive compound) such as ⁹⁰Y, ¹⁷⁷Lu, ¹³¹I, ³²P, ¹⁰B or ²¹³B. The therapeutic compound may also be a toxin, such as B. anthracis edema factor, B. anthracis lethal factor, C. perfringens iota toxin, C. botulinum C2 toxin, C. difficile ADP-ribosyltransferase, C. diphtheriae diphteria toxin fragment A, Burgholderia sp. shiga toxin (subunit A), Clostridium perfringens str. 13 toxin pfoA perfringolysin O, Ricin A chain, plant RIP bouganin, Human RNASE3 ribonuclease (RNase A family, 3) and anthrax lethal factor endopeptidase.

The therapeutic agent could also be any appropriate drug or chemotherapeutic agent. Such agents are well known in the art.

The diagnostic agent could also be a radioactive compound, such as ^{99m}Tc, ¹¹¹In, ⁸²Rb or ²⁰¹Tl. The diagnostic agent could also be a fluorescent compound, preferably GFP, a GFP variant, or a fluorescent small-molecule compound such as FITC (fluorescein isothiocyanate), PE (phycoerythrin), an alexa fluor dye (such as AlexaFluor488 or related dyes) or a cyanine dye (such as Cy3 (Indocarbocyanine) or Cy5 (Indodicarbocyanine) or related dyes). The diagnostic agent may be a molecule capable of mediating bioluminescence, preferably a luciferase molecule, more preferably Gaussia luciferase.

Depending on the type of cargo, the method described above can have a number of different purposes. For example, where the cargo is a therapeutic agent, the method may be a method of treatment of the human or animal body, e.g. a method of treatment of cancer. When the cargo is a diagnostic agent, the method could be an in vivo method of diagnosis.

Preferably, when used in vivo, the binding protein is a Fab, scFV or VHH. These formats result in rapid clearance of the cargo as such fragments rapidly pass through the renal filter. The small size of such fragments also means that rapid tissue penetration occurs.

The method could also be used in vitro when delivering cargo which is either a therapeutic or diagnostic agent.

The method described above provides a number of advantages over known techniques. Use of the binding protein provides greater specificity than when using an un-engineered bispecific antibody and avoids the need to engineer the bispecific antibody to effectively target only cells expressing both target molecules, rather than one. The method of the present invention is also independent of the target molecule. Furthermore, the method allows for fine tuning of the different steps, e.g. depending on the affinity of the targeting molecules for their target molecules, as well as the affinity of the binding protein for the transient PPI.

The dosage regimes will vary depending on the exact system to be used, and could readily be determined by the skilled person. The system should allow for accumulation of the targeting molecules at the desired sites with systemic clearance at non-target sites followed by secondary administration of the binding protein linked to the cargo. The binding protein would accumulate rapidly at the target site due to its interaction with the targeting molecules. Typically, the binding protein linked to the cargo would be administered 2-3 days after administration of the targeting molecules. However, spacing could be anything from hours (e.g. 4, 5, 6 hours) up to 3, 4 or 5 days.

Where the transient PPI is the IL-6/gp80 interaction, it would be appropriate to use a mutated system where interactions with gp130 are attenuated. This could readily be achieved by mutation of residues at the site 2 interface on either 11-6 or gp80. This would minimise any endogenous signalling.

A method for testing whether a binding protein can cross-link two antibody fusions (and thereby their antigens) is described in the Examples. A cancer cell killing assay would also be appropriate for testing a system where a chemotherapeutic agent is to be delivered. Such assays are well known in the art.

### Switching on a Biological Effect Using a Binding Protein Specific for a Junctional Epitope Created by a Transient Protein: Protein Interaction

The invention also provides use of a binding protein as defined above in switching on a biological effect. The biological effect may be switched on solely via administration of the binding protein to a biological system. In other words, the binding protein on its own may act as a molecular switch to switch on the biological effect associated with the transient PPI. Here, when one or two out of the first protein component of the transient PPI, second protein component of the transient PPI and the binding protein are present, the biological effect is not observed. However, when all three of the first and second protein components of the transient PPI and the binding protein are present, and the first and second protein components are co-localised, the PPI is stabilised and the biological effect is induced.

Such a system could have both in vitro and in vivo uses. For example, as discussed further below, stabilisation of the IL-6/gp80 interaction can be used to increase STAT3 phosphorylation (and associated downstream effects). In vivo, such a system has utility for example in liver regeneration after injury (see below).

The present invention also provides a second molecular switching method, where the binding protein is used to switch on a biological effect other than of the transient protein:protein interaction.

In particular, the invention provides a method of switching on a biological effect, comprising:
(1) administering two targeting molecules, wherein:
   the first targeting molecule comprises a first targeting portion which binds specifically to a first target molecule on the target cell and wherein the targeting portion is linked to the first protein component of a transient protein:protein interaction; and
   the second targeting molecule comprises a second targeting portion which binds specifically to a second target molecule on the target cell and wherein the targeting portion is linked to the second protein component of the transient protein:protein interaction; and
(2) administering a binding protein specific for a junctional epitope created by the transient protein:protein interaction, wherein the binding protein is as defined above and wherein administration of the binding protein switches on the biological effect.

This “molecular switch” method is illustrated in FIG. 2.

The biological effect is not limited and could be any effect which is achieved via cross-linking of two target molecules. In an “off” position, with only one or two of the target molecules present (and accordingly only one of the two targeting molecules binding to the target molecule), the biological effect is not observed. However, in an “on” position, where the two target molecules are present and co-located, and the two targeting molecules are bound, addition of the binding protein causes cross linking and activation of the biological effect.

As discussed above, the targeting portions are typically antibodies (or antibody fragments), or ligands, which may be linked to the first and second components of the PPI be any known means, such as using a linker sequence. The target molecules may be antigens or receptors.

The above method may be used as a molecular switch in vitro or in vivo. For example, switching on a biological effect in vivo may be used in the treatment and/or prevention of a disease or condition.

As discussed above, when the transient PPI is the IL-6/gp80 interaction, it would be appropriate to use a mutated system where interactions with gp130 are attenuated.

Example 2 provides a general example of how bispecific targeting which only gives function when two molecular markers are present can be tested.

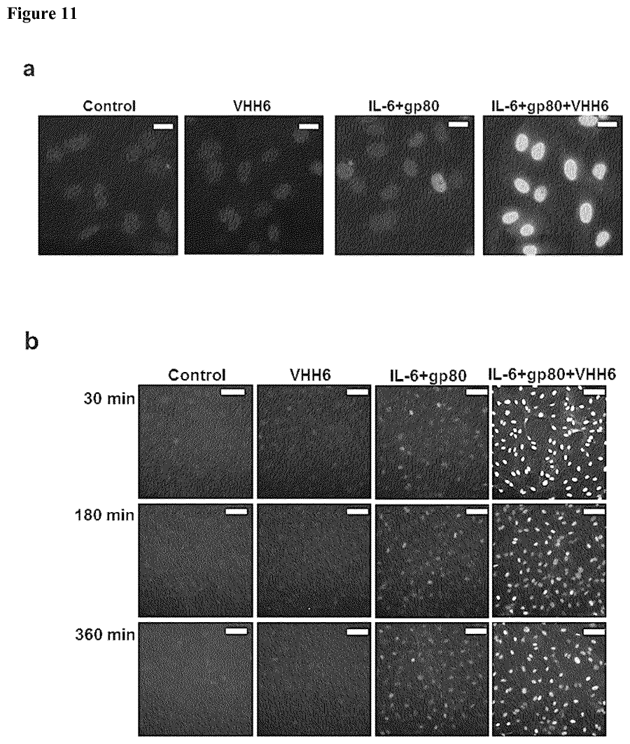

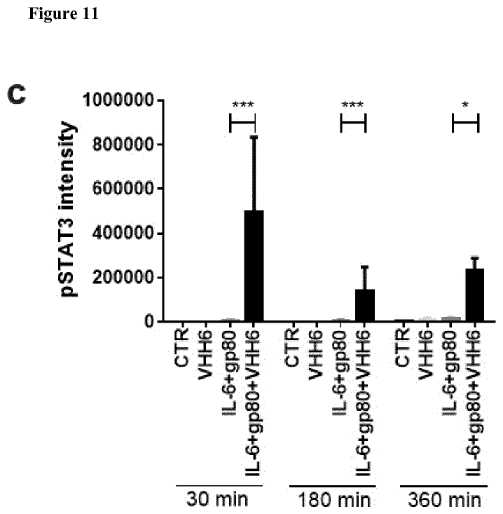

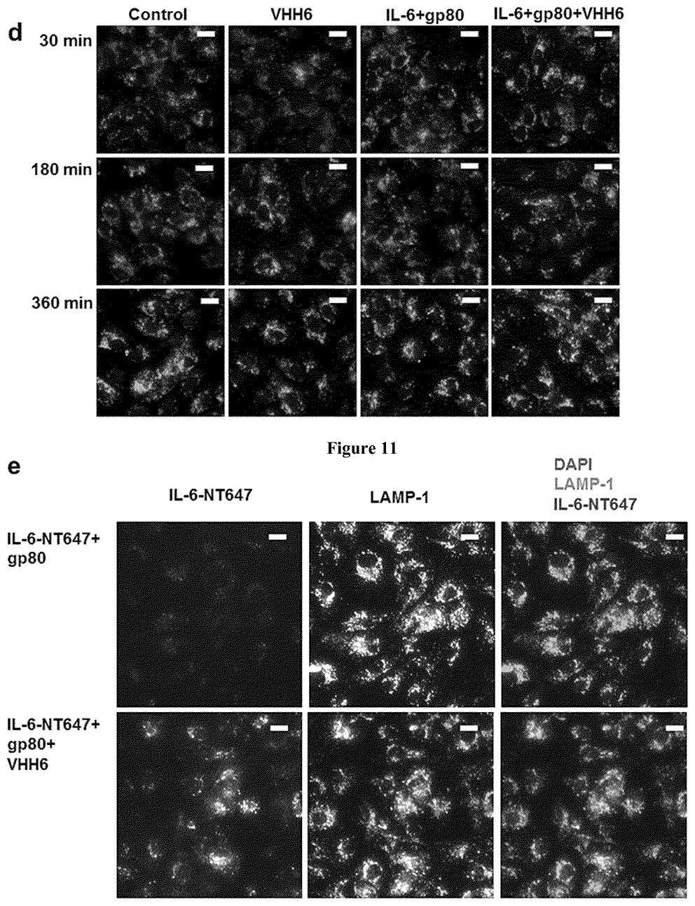

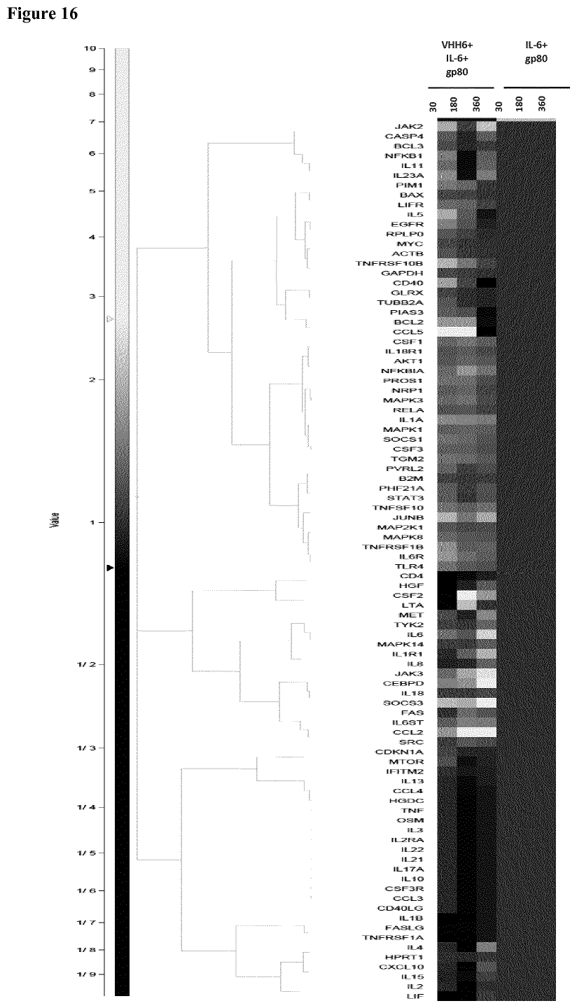

The present invention further provides use of a binding protein as defined above for altering a biological effect. In particular, said biological effect is a signalling response or a biological response. Amplifying the signalling by stabilizing a transient interaction using VHH6 enhances the signalling is demonstrated in particular in FIG. 11. FIG. 16 also demonstrates that said binding proteins modify the biological effect.

In one embodiment, the invention provides a method of altering of a biological effect, comprising:
(1) administering two targeting molecules, wherein:
   the first targeting molecule comprises a first targeting portion which binds specifically to a first target molecule on the target cell and wherein the targeting portion is linked to the first protein component of a transient protein:protein interaction; and
   the second targeting molecule comprises a second targeting portion which binds specifically to a second target molecule on the target cell and wherein the targeting portion is linked to the second protein component of the transient protein:protein interaction; and
(2) administering a binding protein specific for a junctional epitope created by the transient protein:protein interaction, wherein the binding protein is as defined above and wherein administration of the binding protein alters the biological effect.
   More specifically the method provided above is used to amplify, decrease or modify a biological response. More particularly said method is used to switch on/off a biological effect.

### Use of a Binding Protein Specific for a Junctional Epitope Created by a Transient Protein:Protein Interaction in Cross-Linking Target Cells

The invention also provides use of a binding protein as defined above in cross-linking target cells. In the particular, the invention provides a method of cross-linking two target cells using a binding protein, wherein first target cell expresses a first target molecule and the second target cell expresses a second target molecule, and wherein the method comprises:
(1) administering two targeting molecules, wherein:
   the first targeting molecule comprises a first targeting portion which binds specifically to the first target molecule on the first target cell and wherein the targeting portion is linked to the first protein component of a transient protein:protein interaction; and
   the second targeting molecule comprises a second targeting portion which binds specifically to the second target molecule on the second target cell and wherein the targeting portion is linked to the second protein component of the transient protein:protein interaction; and
(2) administering a binding protein specific for a junctional epitope created by the transient protein:protein interaction as defined above.

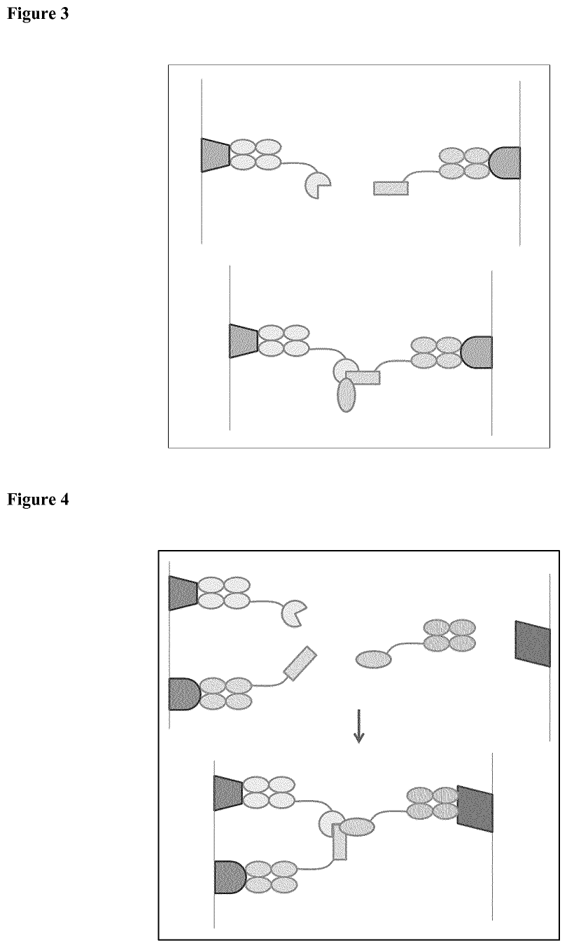

This method is illustrated in FIG. 3.

The invention also provides method of cross-linking two target cells, wherein the first target cell expresses a first and second target molecule and the second target cell expresses a third target molecule, and wherein the method comprises:

(1) administering two targeting molecules, wherein:
the first targeting molecule comprises a first targeting portion which binds specifically to the first target molecule on the first target cell and wherein the targeting portion is linked to the first protein component of a transient protein:protein interaction; and
the second targeting molecule comprises a second targeting portion which binds specifically to the second target molecule on the first target cell and wherein the targeting portion is linked to the second protein component of the transient protein:protein interaction; and

(2) administering a third targeting molecule comprising a third targeting portion which binds specifically to the third target molecule on the second target cell, wherein the targeting portion is linked to a binding protein specific for a junctional epitope created by the transient protein:protein interaction and wherein the binding protein is as defined above.

This method is illustrated in FIG. 4.

Furthermore, the invention provides a method of cross-linking two target cells, wherein the first target cell expresses a first and second target molecule and wherein the second target cell is engineered to present a binding protein on its surface, and wherein the method comprises administering two targeting molecules, where
the first targeting molecule comprises a first targeting portion which binds specifically to the first target molecule on the first target cell and wherein the targeting portion is linked to the first protein component of a transient protein:protein interaction; and
the second targeting molecule comprises a second targeting portion which binds specifically to the second target molecule on the first target cell and wherein the targeting portion is linked to the second protein component of the transient protein:protein interaction;
   wherein the binding protein is as defined above.

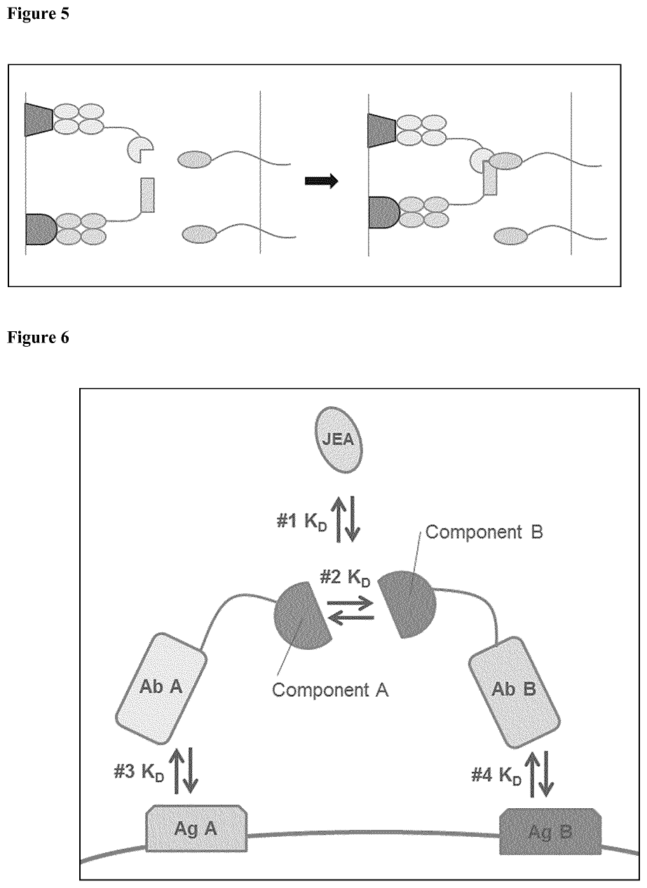

This method is illustrated in FIG. 5.

In all of these methods, the first, second and third (where present) targeting portions may be antibodies or fragments thereof, or ligands for the target molecules. The first, second and third (where present) target molecules may be antigens or receptors.

The first or second target cell is not particularly limited, but could be a cancer cell, which may be cross linked to an effector cell, such as a T cell. Target molecules on cancer cells and T cells would be well known to the person skilled in the art. Exemplary cancer antigens are discussed above. For example, the target molecule on a T cell could be CD3, particularly CD3ε. The target cell could also be an NK (natural killer) cell, where the target molecule may be NKp46. Once again, the method could be an in vivo method, whereby the method is a therapeutic or prophylactic method. The method could also be an in vitro method.

The method can be fine-tuned via the different binding kinetics of the binding protein and of the targeting molecules.

### Medical Uses

Binding proteins of the present invention may have medical applications when administered without targeting molecules. For example, the binding proteins could be used to stabilise the transient PPI in vivo and therefore induce a therapeutic or prophylactic effect (see above). The invention thus provides a method of treatment of the human or animal body comprising administration of a binding protein as described above. The binding protein could also be used to stabilise a transient PPI, before administration of the complex comprising the PPI with binding protein.

As discussed in the Examples of the present application, VHH6 was shown to significantly increase signalling through IL-6/gp80/gp130, culminating in phosphorylation of STAT3. Phosphorylated STAT molecules are translocated to the nucleus, where transcription of genes involved in the response to IL-6 is activated. VHH6 was indeed shown to affect transcription of genes in the IL-6 pathway. VHH6 thus seems to modulate disease states and therefore offers potential in further understanding the role of IL-6 e.g. in inflammation.

VHH6 (or indeed other binding proteins specific for a junctional epitope created by the IL-6/gp80 interaction) could also be used therapeutically where IL-6 has a role as growth factor, such as in tissue regeneration (particularly after injury in liver regeneration). For example, the binding protein could be used as a treatment to promote liver regeneration and restore liver function. Liver injury may be caused by a toxic substance (e.g. drug induced hepatopathy or alcoholic hepatitis), mechanical trauma, malignancy, an autoimmune disease or by a pathogen (e.g. hepatitis virus). The binding protein is administered in a therapeutically effective amount, which could be determined by the skilled person. The binding protein is typically administered in a pharmaceutical composition as discussed below.

Such a binding protein could also be used in vitro in the activation of hepatocyte-like progenitor cells and in expansion of hematopoietic progenitor cells.

Galun (2013) Methods in Molecular Biology, 982, 59-77 considers the regenerative role of IL-6. Wuestefeld et al (2003) The Journal of Biological Chemistry, 278, 11281-11288 considers the role of the IL-6/gp80 pathway in liver regeneration, Best et al (2010) Experimental and Molecular Pathology, 88, 7-14 looks at hepatocyte-like progenitor cells in rat liver injury and Fischer (1997) Nature Biotechnology, 15, 142-145 considers hematopoietic progenitor expansion. The use of covalently linked IL-6/gp80 has also been described for tissue regeneration (particularly liver) in WO 99/62534.

Use of a Binding Protein Specific for a Junctional Epitope Created by a Transient Protein. Protein Interaction in Tissue Regeneration.

Importantly, the present invention allows to target biological effects to the sites of the two targets described above to avoid systemic effects that might occur if one of these targets was administered systemically. Systemic administration of IL-6 could cause broad inflammation which would be undesirable (Gabay et al. Arthritis Res Ther 8, suppl. 2:S3 (2006)). Using the binding proteins disclosed herein, effects could be restricted to the site of liver injury where gp80 and IL-6 are released. The binding proteins disclosed herein could be used to enhance optic nerve regeneration by promoting IL-6 signalling (Fischer, Eye 31, 173-178 (2017)), in airway tissue regeneration (Tadokoro et al. PNAS 111(35), E3641-E3649 (2014)) or more broadly in other areas of regenerative medicine.

In particular, the present invention provides a method of amplifying or increasing the IL-6/gp80 signalling, comprising administering a binding protein of the invention described herein. Said method might be practiced as an in vitro method or an in vivo method.

In particular, the present invention provides a method of tissue regeneration, comprising administering a binding protein of the invention described herein. In a particular embodiment said method is an in vitro method. In yet another embodiment said method is an in vivo method.

In particular, the present invention provides a method of tissue regeneration, comprising administering a binding protein of the invention described herein. Said protein might be administered in a pharmaceutical composition as disclosed further herein.

The present invention provides further the use of the binding protein of the invention for tissue regeneration.

In particular embodiment said protein is a protein specific for a junctional epitope created by a transient protein:protein interaction, wherein said protein:protein interaction is the IL-6/gp80 interaction. More specifically said protein amplifies or increases the IL-6/gp80 signalling by stabilizing a transient interaction.

More particular said tissue regeneration is liver regeneration. In another embodiment said tissue regeneration is nerve regeneration. In yet another embodiment said tissue regeneration is airways regeneration.

More specifically said binding protein is the VHH6 antibody of the invention. More particular said binding protein is a humanized VHH6 antibody. More specifically said humanized VHH6 antibody is the humanized VHH6 antibody as disclosed herein.

### Pharmaceutical Composition

A binding protein of the invention may be provided in a pharmaceutical composition. The pharmaceutical composition will normally be sterile and will typically include a pharmaceutically acceptable carrier and/or diluent. A pharmaceutical composition of the present invention may additionally comprise a pharmaceutically acceptable adjuvant and/or carrier.

As used herein, “pharmaceutically acceptable carrier” includes any and all solvents, dispersion media, coatings, antibacterial and antifungal agents, isotonic and absorption delaying agents, and the like that are physiologically compatible. The carrier may be suitable for parenteral, e.g. intravenous, intramuscular, intradermal, intraocular, intraperitoneal, subcutaneous, spinal or other parenteral routes of administration, for example by injection or infusion. Alternatively, the carrier may be suitable for non-parenteral administration, such as a topical, epidermal or mucosal route of administration. The carrier may be suitable for oral administration. Depending on the route of administration, the modulator may be coated in a material to protect the compound from the action of acids and other natural conditions that may inactivate the compound.

The pharmaceutical compositions of the invention may include one or more pharmaceutically acceptable salts. A “pharmaceutically acceptable salt” refers to a salt that retains the desired biological activity of the parent compound and does not impart any undesired toxicological effects. Examples of such salts include acid addition salts and base addition salts.

Pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. In many cases, it will be desirable to include isotonic agents, for example, sugars, polyalcohols such as mannitol, sorbitol, or sodium chloride in the composition.

Therapeutic compositions typically must be sterile and stable under the conditions of manufacture and storage. The composition can be formulated as a solution, microemulsion, liposome, or other ordered structure suitable to high drug concentration.

Pharmaceutical compositions of the invention may comprise additional active ingredients.

Also within the scope of the present invention are kits comprising the binding proteins of the invention and instructions for use.

The binding proteins of the invention or formulations or compositions thereof may be administered for prophylactic and/or therapeutic treatments.

In therapeutic applications, compounds are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”.

In prophylactic applications, formulations are administered to a subject at risk of a disorder or condition as described above, in an amount sufficient to prevent or reduce the subsequent effects of the condition or one or more of its symptoms. An amount adequate to accomplish this is defined as a “prophylactically effective amount”. Effective amounts for each purpose will depend on the severity of the disease or injury as well as the weight and general state of the subject.

A subject for administration may be a human or non-human animal. The term “non-human animal” includes all vertebrates, e.g., mammals and non-mammals, such as non-human primates, sheep, dogs, cats, horses, cows, chickens, amphibians, reptiles, etc. Administration to humans is typical.

A pharmaceutical composition of the invention may be administered via one or more routes of administration using one or more of a variety of methods known in the art. As will be appreciated by the skilled artisan, the route and/or mode of administration will vary depending upon the desired results. Examples of routes of administration for compounds or pharmaceutical compositions of the invention include intravenous, intramuscular, intradermal, intraocular, intraperitoneal, subcutaneous, spinal or other parenteral routes of administration, for example by injection or infusion. The phrase “parenteral administration” as used herein means modes of administration other than enteral and topical administration, usually by injection. Alternatively, compound or pharmaceutical composition of the invention can be administered via a non-parenteral route, such as a topical, epidermal or mucosal route of administration. The pharmaceutical composition of the invention may be for oral administration.

A suitable dosage of pharmaceutical composition of the invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A suitable dose may be, for example, in the range of from about 0.01 μg/kg to about 1000 mg/kg body weight, typically from about 0.1 μg/kg to about 100 mg/kg body weight, of the patient to be treated. For example, a suitable dosage may be from about 1 μg/kg to about 10 mg/kg body weight per day or from about 10 μg/kg to about 5 mg/kg body weight per day.

Dosage regimens may be adjusted to provide the optimum desired response (e.g., a therapeutic response). For example, a single dose may be administered, several divided doses may be administered over time or the dose may be proportionally reduced or increased as indicated by the exigencies of the therapeutic situation. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Administration may be in single or multiple doses. Multiple doses may be administered via the same or different routes and to the same or different locations. Alternatively, doses can be via a sustained release formulation, in which case less frequent administration is required. Dosage and frequency may vary depending on the half-life of the antagonist in the patient and the duration of treatment desired.

As mentioned above, pharmaceutical composition of the invention may be co-administered with one or other more other therapeutic agents.

Combined administration of two or more agents may be achieved in a number of different ways. Both may be administered together in a single composition, or they may be administered in separate compositions as part of a combined therapy. For example, the one may be administered before, after or concurrently with the other.

### Method of Producing an Antibody Specific for a Junctional Epitope Created by a Transient Protein:Protein Interaction

The invention also provides a method of producing an antibody specific for a junctional epitope created by a transient protein:protein interaction. The method involves immunising an animal with a fusion protein of the stabilised PPI complex. Suitable animals are described above, but are may usefully be camels in order to generate VHH antibodies.

With regards to the fusion protein, for example, one of the protein components of the PPI may be fused directly onto the terminus (e.g. the C-terminus) of the other protein as described in the Examples for generation of VHH6. Methods of fusing proteins together are well known in the art.

The method then involves selecting for an antibody which interacts with the complex of the PPI but not with each individual component of the PPI in the absence of the other. Methods of determining whether an antibody interacts with the PPI complex, but not with each individual component, are described above and include ELISA, HLPC, MicroScale Thermophoresis, Octet (Biol-Layer Interferometry) and surface plasmon resonance (typically at 25° C.). In the present invention, the binding protein may show undetectable binding to each individual component of the PPI using any one, preferably multiple, more preferably all, of the above techniques. As discussed above, the binding protein may in some instances show a very low level of binding to the individual proteins of the PPI, but not enough to limit the usefulness of the binding protein.

The method may involve further steps, such as determining that the binding protein stabilises the protein:protein interaction. Methods and representative values for stabilisation of the PPI are discussed above.

The method may for example also use additional techniques, such as X-ray crystallography to confirm the junctional nature of the binding protein. Junctional epitopes are described above.

Use of a Binding Protein Specific for a Junctional Epitope Created by a Transient Protein:Protein Interaction in Screening for a Substance which Modulates Signalling Through a Protein:Protein Interaction

The invention further provides use of a binding protein of the invention in screening for substances which modulate signalling through a transient PPI. The substance may for example be a small molecule, peptide or even an antibody. The substance is typically an inhibitor i.e. a substance which reduces signalling through the transient PPI. In other words, the binding protein may be used in structure-aided drug discovery. Such analysis may be computational.

The following Examples illustrate the invention. Using the interaction between IL-6 and gp80 as a well-known example of a PPI, the present inventors have produced and characterized VHH6, a prototype locking (junctional) antibody. VHH6 specifically recognizes an epitope that spans the junction between IL-6 and gp80, locking them together in a stable functional unit. Such an antibody finds use in a number of different applications, including specific targeting of antigens on cancer cells.

### EXAMPLES

### Example 1: Production and Testing of VHH6

### Materials and Methods—Antibody Production

Protein production and purification: With the exception of Human HyperIL-6 (Fischer, M. et al. I. Nature biotechnology 15, 142-145 (1997)), all proteins (FusionIL-6, gp80V4, gp80-hscFc, IL-6 and IL-6S21) were designed in house. FusionIL-6 is a modified version of HyperIL-6 where all non-natural linker residues have been removed, IL6 (V30-M212) was fused directly onto the C-terminus of gp80 at the end of domain 3 (S320). In addition, two unpaired cysteines were mutated to alanine (C211A, C277A). To increase success in crystallization, an engineered from of gp80 was used (gp80V4). In this construct the N-terminal 14aa of gp80 domain 1 (L20-G33) were fused to the N-terminus of gp80 domain 2 using a short linker. The cysteine (C25) in the domain 1 fragment forms a disulphide bond with cysteine 193 in domain 2 stabilising the protein. In addition gp80V4 also has unpaired cysteines C211 and C277 mutated to alanine.

All those proteins were produced in house. For constructs, vectors and cell systems used for protein production see Table 1 below:

TABLE 1
Constructs used in protein production

| Construct | Expression Host | Promoter | Affinity Tag | Expressed Protein |
|---|---|---|---|---|
| pNAFL-8His-fusionIL-6(gp80D123) | CHO | CMV | 8His | gp80(L20-S320)(C211A, C277A)-IL-6 |
| | | | | (V30-M212) |
| pNAFL-gp80D123-hscFc | CHO | CMV | Single chain human | gp80 (M1-P322)(C211A, C277A)-hscFc |
| | | | IgG Fc fragment | gp80(L20-G33- |
| pNAFL-8His-gp80delta1, 2, 3 V4 | CHO | CMV | 8His | AGAG linker-D111-P322(C211A, C277A) |
| pTrx-6His-hIL-6 | E. coli | T7 | Thioredoxin-6His | Trx-6His-IL-6(A28-M212) |
| pTrx-6His-IL-6(S21) | E. coli | T7 | Thioredoxin-6His | Trx-6His-IL-6(S49-M212) |
| pIMMs-6His-VHH6 | Expi-HEK | CMV | 6His | VHH6-6His |

Numbering for gp80 is based on Uniprot reference P08887.

Protein used for crystallography and HDX-MS were produced in Expi-HEK or CHO cells (Gibco, Thermo Fisher Scientific) in media containing Kifunensine (GlycoSyn). IL-6 was produced in E. coli. For in vitro experiments IL-6, gp80 were purchased from R&D Systems. For SPR experiments gp130 was purchased from R&D Systems.

Ni purification: Clarified supernatant was passed through a 0.22 μm filter and loaded onto a 1 ml/5 ml Ni Excel column (GE Healthcare) pre-equilibrated in PBS (pH 7.4) containing 20 mM imidazole. Bound protein was eluted with PBS (pH 7.4) containing 250 mM imidazole. Fractions containing protein of interest as judged by 4-12% Bis/Tris NuPage (Life Technologies, Gibco, Thermo Fisher Scientific) gel electrophoresis were dialyzed to remove imidazole and treated with TEV protease at a ratio of 1 mg per 100 mg protein. After 0/N incubation at 4° C. the sample was passed over a Ni Excel column for a second time removing non-cleaved protein and His tagged TEV protease resulting in pure cleaved protein in the column flow through.

Fc-tagged protein purification: Clarified supernatant was passed through a 0.22 μm filter and loaded onto a 1 ml/5 ml Mab Selectsure column (GE Healthcare) pre-equilibrated in PBS (pH 7.4). If the protein was mouse Fc-tagged, 0.5 M NaCl was added to the supernatant to facilitate binding. Protein was then eluted with a phosphate/citrate pH gradient using 0.15 M Na₂HPO₄ at pH 9 and 0.1 mM Sodium Citrate at pH 2. Proteins were eluted at around pH 3.5 and immediately neutralized using 2 M Tris at pH 8.

TEV-HuFc tagged proteins were eluted by adding TEV protease directly to the Mab Selectsure beads at 100 μg TEV per ml of beads and incubating in PBS at 4° C. overnight. Beads were then removed by centrifugation to leave cleaved untagged protein.

Gel Filtration: Proteins were concentrated using Centricon Tubes (Merck Millipore) and loaded onto an S75 26/60 gel filtration column (GE Healthcare) pre-equilibrated in PBS. Peak fractions were pooled and concentrated Centricon Tubes (Merck Millipore).

Antibody discovery campaign: Two camels were immunized subcutaneously over a period of three months with HyperIL-621, escalating the dose from 3 μg up to maximum 1 mg in half log increments, at the Central Veterinary Research Laboratory (CVRL) in Dubai, according to a humane protocol approved by the Scientific Directorate of the CVRL. Experimental procedures were performed in accordance with relevant guidelines/regulations and approved by the Scientific Directorate of the CVRL. Initial immunization was carried out with complete Freund's adjuvants, while the subsequent boosts used incomplete Freund's adjuvants. 3 mL serum samples were collected at 8, 10, 12, and 18-week post-initial immunization to monitor the immune response; at 19 week, 200 mL of heparinized blood were collected and PBMCs isolated and frozen. Single domain VHHs were generated from frozen PBMCs from immunized animals by cloning variable region genes from isolated B cells using the antibody discovery platform described in Tickle, S. et al. JALA 14, 303-307 (2009) and Lightwood, D. et al. Journal of molecular biology 425, 577-593 (2013). VHHs were subsequently cloned in pIMMs vector with TEV-6His tag downstream the VHH fragment for mammalian expression (Hancock, L. M. A study of camelid antibodies reveal structural features of a cytokine. PhD Thesis University of London (2011)).

### Materials and Methods—Preliminary Screening Assays

ELISA—IL-6, gp80 and FusionIL-6 used in ELISA to coat plates were diluted to a final concentration of 2 mg/mL in carbonate buffer (0.16% Na₂CO₃ and 0.3% NaHCO₃ in H₂O). Coated plates were incubated at 4° C. overnight. The following day, the plates were washed twice in PBST, blocked in PBS/5% BSA for 2 h at RT, and again washed twice in PBST. For preliminary screening, either supernatant from HEK cells or purified VHHs was added. Anti-llama-HRP or anti-His-HRP antibody was added for 1 h at RT. Afterwards the plates were washed twice with PBST and developed with TMB substrate. The reaction was stopped by adding NaF. Plates were read at 650 nm with a reference wavelength of 450 nm (Biotek PowerWave HT Microplate Spectrophotometer). In other experiments, IL-6 was coated overnight on the plate; VHH6 was added alone or with gp80-hscFc. Alternatively, gp80-hscFc was coated overnight on the plate, VHH6 was added alone or with IL-6. Anti-llama-HRP antibodies (Bethyl Laboratories), anti-human Fc-HRP (Jackson Lab, USA) or anti-His-HRP (Qiagen) antibodies were used for revealing the plate.

HPLC—Solutions VHH6, gp80 and IL-6 alone or in combination were incubated overnight at 4° C. The following day, size exclusion chromatography was carried out on a HiLoad 16/60, Superdex G3000 column (GE Healthcare) equilibrated with 50 mM NaCl, 25 mM Tris, 5% (v/v) glycerol using an Agilent 1100 Series column (GE Healthcare).

MST—MicroScale Thermophoresis (MST) relates to the movement of particles subjected to a temperature gradient. When a temperature gradient is applied, changes of the hydration shell of proteins reflect in protein structure/conformation changes and this can be used to determine receptor:ligand binding affinities (Jerabek-Willemsen, M., Wienken et al. Assay and drug development technologies 9, 342-353 (2011). Both IL-6 and gp80 were labelled with NT647 using the Monolith labelling Red NHS kit (NanoTemper Technologies GmbH, Germany). NT647 dye is optimized for MST and carries a reactive NHS-ester group that modifies primary amines in lysine. Labelling and purification was carried out according to the protocol provided by the supplier. Standard treated capillaries NT.115 Series (NanoTemper Technologies GmbH, Germany) were used throughout the experiments. The red channel of Monolith NT.115 Blue/Red Monolith NT.115 (NanoTemper Technologies GmbH, Germany) was used. Both IL-6 and gp80 were labelled with NT647 dye with a ratio protein to dye above 0.5 to check for binding of IL-6 or gp80 only; a titration experiment using up to 20 mM VHH6 was used. To determine the binding between VHH6 and IL-6−gp80, 10 nM IL-6NT647 and 20 nM gp80 was used, and VHH6 was titrated in, at a concentration started with at least ten-fold the concentration of labelled molecules.

Octet—Octet is based on Bio-Layer Interferometry (BLI) for measuring e.g. protein:protein interaction and is a label-free technique. BLI analyzes the interference pattern of white light reflected from two surfaces: a layer of immobilized protein on the biosensor tip, and an internal reference layer. Bio-Layer Interferometry (BLI) was used as a label-free technology for protein:protein binding (Abdiche et al Journal of immunological methods 382, 101-116 (2012)). The binding between VHH6 immobilized on the biosensor tip surface using Biosensor NiNTA tips (ForteBio, Pall Corporation) and FusionIL-6, IL-6, gp80 and IL-6−gp80 in complex was measured. Interference patterns for the binding to or dissociating from the biosensor were measured in real time to generate a response profile on the Octet® System Octet 384 Red (ForteBio, Pall Corporation).

SPR measurements—All SPR experiments were carried out at 25° C. in HBS-P+ buffer (10 mM HEPES pH 7.4, 0.15 M NaCl, 0.05% (v/v) surfactant P20, (GE Healthcare) as the running buffer. VHH6, IL-6, gp80 and gp130 proteins were immobilized on CMS sensor chips (GE Healthcare) by standard amine coupling method, as recommended by the manufacturer.

Qualitative SPR analysis was performed using a BIAcore 3000 (GE Healthcare); gp80 and VHH6 were injected at 50 nM concentration at a flow rate of 30 μl/min. gp80 was injected for 180 s and the dissociation was monitored for 360 s. Alternatively, gp80 was injected for 180 s and subsequently VHH6 was co-injected and the dissociation was monitored for 360 s.

Quantitative SPR analysis was carried out using a BIAcore T200 (GE Healthcare). The binding measurements were performed at a flow rate of 100 μl/min. The protein of interest was injected for 180 s and the dissociation was monitored for 360 s. Binding of IL-6, gp80 and VHH6 were individually tested in a concentration series (0-250 nM, as two-fold serial dilution). When proteins were tested in combinations, i.e. IL-6 and gp80, IL-6 and VHH6, gp80 and VHH6, gp80 and IL-6 and VHH6, one of the protein was titrated in a concentration series (0-250 nM), while the other was kept constant at an excess concentration of 2 μM to ensure complex formation. The sensor surface was regenerated with 4 mM MgCl₂. Background subtraction binding curves were analyzed using the T200 evaluation software (version 1.0) following standard procedures. Due to the complexity of the interactions studied, the data analysis was limited to the most informative, in this case k_{d} parameter, which was determined by fitting a single exponential decay model to dissociation data within 360 s range.

In order to evaluate the effect of VHH6 on the binding of the IL-6−gp80 complex to gp130, the kinetic data were fitted by applying a 1:1 binding model. The affinities for the interactions that reached equilibrium during the injection time of the experiment were determined from a steady state affinity fit, and are reported in the Supplementary Tables. The binding curves were re-plotted in Prism (GraphPad Software Inc.) to render the figures with a better resolution.

Mutant versions of VHH6 containing single alanine substitutions at positions 27, 32, 74, 101 and 113, and double alanine substitutions at positions 27/32, 74/101 and 113/101 were generated and supernatants produced in transient transfected cells (see paragraph Generation of VHH6 mutants) were tested by SPR. SPR analysis was performed using a BIAcore 3000 (GE Healthcare). His-tagged VHH6 and VHH6 mutants (1:5 diluted supernatants) were captured on a NTA chip (30 μl at 10 μl/min) following charging of the surface by injection of 10 μl 0.5 M NiCl₂. An equimolar mixture of IL-6 and gp80 (50 nM each) was injected for 300 s at a flow rate of 10 μl/min followed by a dissociation phase of 300 s. At the end of each cycle the chip surface was regenerated by injection of 350 mM EDTA (2×10 μl).

Cell culture—Pooled HUVEC cells were purchased from Gibco (Gibco, Thermo Fisher Scientific) and grown in 200PRF media supplemented with Low Serum Supplement Media (Gibco, Thermo Fisher Scientific). Different batches of HUVEC cells were tested in our experiments in order to have a good population representation. Cells were not used beyond the 5-6th passage.

Phospho-STAT3 staining—Cells were plated the day before at 1×10⁴ cells/well in 96 well BD Falcon Becton Black/Clear tissue culture treated plates (Becton Dickinson). The following day, the cells were treated with FusionIL-6 (3 ng/mL) and IL-6+gp80 alone (respectively 10 ng/mL and 20 ng/mL) or in combination with an excess of VHH6 and fixed with PFA 4% at chosen time points (30 min, 180 min and 360 min). After 20 min incubation at RT, the cells were washed twice with PBS and ice-cold methanol was added and stored at −20° C. Before staining, the cells were washed twice in PBS, incubated for 20 min at 4° C. with PBS/10% FCS. Afterwards, anti-Phospho-STAT3 (Tyr705) (clone D3A7, Cell Signalling) was added at a dilution suggested by the manufacturer (1:100 in PBS/10% FCS) and incubated for 1 h at 4° C. After washing twice in PBS, secondary antibody goat anti-rabbit-AlexaFluor568 (Gibco, Thermo Fisher Scientific) was added and incubated for 1 h at 4° C. Then cells were washed twice in PBS, and nuclei stained for 10 min with a solution of DAPI in PBS/10% FCS (final concentration 2 mg/mL). Finally, cells were washed twice with PBS and 200 μL of fresh PBS were added to each well and stored in the dark at 4° C.

Internalization of IL-6−gp80 complex—Cells were plated the day before at 1×10⁴ cells/well in 96 well BD BIOCOAT Cell Environment Collagen Cellware Black/Clear plates (Becton Dickinson). The following day the cells were treated with IL-6-NT647 alone (100 ng/mL), IL-6-NT647+gp80 (respectively 100 ng/mL and 200 ng/mL) with and without the addition of an excess of VHH6 and fixed with PFA 4% at chosen time points. After 20 min incubation at RT, the cells were washed twice with PBS and ice-cold methanol was added and stored at −20° C. Before staining, the cells were washed twice in PBS, incubated for 20 min at 4° C. with PBS/10% FCS. Afterwards, mouse anti-LAMP1 antibody (clone H4A3, Abcam) was added at a dilution suggested by the manufacturer (1:100 in PBS/10% FCS) and incubated for 1 h at 4° C. After washing twice in PBS, secondary antibody goat anti-mouse-AlexaFluor488 (Gibco, Thermo Fisher Scientific) was added and incubated for 1 h at 4° C. Then cells were washed twice in PBS, and nuclei stained for 10 min with a solution of DAPI in PBS/10% FCS (final concentration 2 mg/mL). Finally, cells were washed twice with PBS and 200 μL of fresh PBS were added to each well and stored in the dark at 4° C.

Image Acquisition and Analysis—Images were acquired using Cellomics Arrayscan (Thermoscientific). For each well, 16-fields were captured containing approximately 100 cells per field at a resolution of 1104×1104 pixels. DAPI, Alexa488, Alexa568 and Alexa647 were detected using appropriate excitation and emission filter sets. Exposure times were kept constant between repeated experiments.

Cells were analyzed using the Cellomics Spot Detector Algorithm. Nuclei were detected on the basis of DAPI staining and STAT3 intensity measured within this region. The ‘spot total intensity per object’ parameter was used for further analysis. For analysis of internalized IL-6-NT647, the mask for analysis was extended from the nuclei and NT-647 intensity measured from spots detected in this region.

Statistical analysis—The raw data from the Arrayscan analysis were processed using the ‘spot total intensity per object’ parameter, and this parameter from three replicates was statistically analyzed using a linear mixed model for repeated measurements using the software package SAS 9.2 (SAS Institute, Cary N.C.).

qPCR array—Briefly, HUVEC cells were seeded at a concentration of 5×10⁵ cells/well in 6-well plates (Falcon, BD Biosciences) the day before. The following morning, the cells were treated with gp80+IL-6 and VHH6+80-+L-6 at the same concentration used for the pSTAT3 staining for 30, 180 and 360 min. At different time points, cells were washed twice with PBS, re-suspended in lysis buffer (Qiagen) and immediately frozen in dry ice and stored at −80° C.

Total RNA was extracted from HUVEC cell lysates using the RNeasy Plus Mini Kit (Qiagen) and quantitated using a Nanodrop (Thermo Fisher). Using the RT² First Strand cDNA Kit (SABiociences, Qiagen) 400 ng of mRNA was converted to cDNA according to the manufacturer's protocol. The ‘Human IL-6/STAT3 Signalling Pathway Plus’ (PAHS-160YE-4) array designed to quantitatively measure mRNA levels of 84 key genes involved IL-6 signaling, 384 well format, were run on an ABI 7900HT machine. Data were analysed using the RT2 Profiler PCR array web based data analysis template v3.5 (http://pcrdataanalysis.sabiosciences.com/pcr/arrayanalysis.php) and changes in gene expression changes were calculated using the ΔΔCₜ method with normalization of the raw data to housekeeping genes. Each RT² Profiler PCR Array plates contained 84 genes related to IL-6 pathway plus 5 different housekeeping genes. Three biological independent replicas of the same experiments were analyzed together. Only statistical significant results (p≤0.05) were showed alongside with p-value.

### Materials and Methods—Structure Determination

X-ray crystallography—The IL-6, gp80 and VHH6 complex was prepared using the engineered gp80V4 construct (described in protein production and purification). The additional domain 1 residues for gp80 were necessary as they make a range of intramolecular interactions to domain 2, as observed in the isolated gp80 X-ray structure (PDB entry 1N26). The majority of the domain 1 residues are not seen in the X-ray structure due to molecular disorder although the cysteine disulphide bond (Cys25-Cys193) is still retained and is clearly visible. The purified VHH6, gp80 and IL-6 were mixed in a molar ratio of 2:1:1 and incubated overnight at 4° C. to allow the complex formation. The complex was purified by size exclusion chromatography (SEC) over a HiLoad 16/60, Superdex 200 column (GE Healthcare) equilibrated with 50 mM NaCl, 25 mM Tris, 5% (v/v) glycerol. Fractions containing the complex were pooled and concentrated to 9 mg/ml for crystallography. Conditions suitable for crystal growth were identified by the sitting drop vapor diffusion method using commercially available crystallization screens (Qiagen). To generate diffraction quality crystals, hanging drop vapor diffusion method was used. 1 μl of protein solution was mixed with 1 μl of reservoir solution containing 0.1 M MES, pH 6.5, 14% PEG20K. Crystals reached full size in 14 d at 19° C. Crystals were harvested and flash frozen in liquid nitrogen without additional cryo-protectant. Diffraction data to 2.7 Å was collected from a single crystal on the Proximal beamline at Soleil Synchrotron, France, and processed using XDS (Kabsch, W. Xds. Acta crystallographica. Section D, Biological crystallography 66, 125-132 (2010)). The structure of the complex was solved by molecular replacement with Phaser (McCoy, A. J. et al. Journal of applied crystallography 40, 658-674 (2007)), using coordinates of an in-house VHH structure, and IL-6 and gp80 from PDB entry 1P9M (Boulanger M. J. et al. Science 300, 2101-2104 (2003)), as search models. The initial structure was refined with iterative cycles of simulated annealing, energy minimization and manual rebuilding using CNS (Brunger, A. T. et al. Crystallography & NMR system: A new software suite for macromolecular structure determination. Acta crystallographica. Section D, Biological crystallography 54, 905-921 (1998) and Brunger, A. T. Version 1.2 of the Crystallography and NMR system. Nature protocols 2, 2728-2733 (2007)) and COOT (Emsley, P. & Cowtan, K. Coot: model-building tools for molecular graphics. Acta crystallographica. Section D, Biological crystallography 60, 2126-2132 (2004)). Model geometry was validated using Molprobity (Chen, V. B. et al. MolProbity: all-atom structure validation for macromolecular crystallography. Acta crystallographica. Section D, Biological crystallography 66, 12-21 (2010)). Surface areas of the protein interfaces were calculated using PISA (Krissinel, E. & Henrick, K. Inference of macromolecular assemblies from crystalline state. Journal of molecular biology 372, 774-797 (2007)). Molecular visualizations were generated with Pymol (DeLano W., The PyMOL Molecular Graphics System., 2002, version 1.7, DeLano Scientific LLC, San Carlos, Calif. www.pymol.org). Data collection and refinement statistics are summarized below:

TABLE 2
Data collection and refinement statistics

| | Junctional Epitope | |
|---|---|---|
| | Antibody in complex | |
| | IL-6 and gp80 | |
| Data collection | | |
| Space group | C 1 2 1 | |
| Cell dimensions | | |
| a, b, c (Å) | 249.03, 67.80, 78.16 | |
| α, β, γ (°) | 90.00, 104.53, 90.00 | |
| Resolution (Å) | 47.84-2.70 | (2.86-2.69) * |
| Rmeas (%) | 7.2 | (50.1) |
| CC_{1/2} (%) | 99.9 | (91.5) |
| I/σI | 20.88 | (3.97) |
| Completeness (%) | 99.4 | (96.9) |
| Redundancy | 7.5 | (7.5) |
| Refinement | | |

| Resolution (Å) | 20.00-2.70 |
|---|---|
| No. reflections | 34,870 |
| R_{work}/R_{free} | 0.2313/0.2872 |
| No. atoms | |
| Protein | 7590 |
| Water | 111 |
| B-factors | |
| Protein | 56.33 |
| Water | 43.19 |
| R.m.s. deviations | |
| Bond lengths (Å) | 0.007 |
| Bond angles (°) | 1.289 |

* Values in parentheses are for highest-resolution shell.

Generation of VHH6 mutants—To investigate the contribution to binding of IL-6 and gp80 by VHH6 residues identified in the crystal structure as being likely contact residues, we generated a panel of mutants using oligonucleotide-directed mutagenesis. Following small scale transient expression in ExpiHEK cells, supernatants were analyzed by SPR. The kd generated using supernatants from the single and double mutants were compared to the supernatant from VHH6.

HDX-MS—40 mM gp80V4-IL-6 or 40 mM 80V4-IL-6-VHH6 complex, both purified by SEC, were diluted 20× either into 10 mM phosphate in H₂O (pH 7.0), or into 10 mM phosphate in D₂O (pD 7.0) and left to react for 0.5, 2, 10, 30 or 120 min. After reaction, these were diluted 1:1 with a cold quench solution (1° C., 3.4 M Gdn.HCl, 500 mM TCEP, 200 mM phosphate) to give a final pH of 2.5. This mixture was immediately injected into the nanoAcquity HDX module (Waters Corp.) for peptic digest. All liquid handling steps were performed by a LEAP-PAL robot (CPC). The protein samples were washed onto the Enzymate pepsin column (25° C.) with 0.1% HCOOH in H₂O at a flow-rate of 90 mL×min⁻¹, and resulting peptides trapped on a VanGuard C18 trapping column over 3 min. These peptides were washed off the trap-column by reversing the flow and separated over a BEH C18 column (10×1.0 cm, 1.7 μm), using the following gradient: 0 min, 5% B; 7 min, 40% B; 8 min, 95% B; 10 min, 95% B, 11 min, 5% B (B: 0.2% HCOOH in H₂O, A: 0.1% HCOOH in MeCN). Data acquisition was performed on a Synapt G2S (Waters Corp.) run in Tof-only mode over an m/z range of 50-2000 Th, using an MSe method (low collision energy, 10V; high collision energy: ramp from 15V to 35V). Glu-1-Fibrinopeptide B peptide was used for internal lock mass correction.

HDX-MS Data Processing—Data were collected in triplicate, with blanks run between each data-point. MSe results from the H₂O-diluted sample were analyzed using PLGS v3.0.1, searching for peptides against a database of the IL-6 and gp80V4 sequences only, with precursor intensity threshold of 500 counts and 3 matched product ions required for assignment. Ion accounting files for the 6 control samples were combined into a peptide list imported into Dynamx v2.0 software, which was further curated to disallow peptides with low energy intensity less than 100, peptides longer than 25 amino acids, and peptides eluting with a retention time longer than 9 minutes. Dynamx v2.0 was used to quantify the isotopic envelopes resulting from deuterium uptake for each peptide at each time-point; these data were then interrogated manually to ensure correct assignment of m/z peaks. Students t-tests were performed on the triplicate data-points for each time-point, and the significance of differential HDX between gp80V4-IL-6 and gp80V4-IL-6-VHH6 being defined as p<0.01.

### Results

### VHH6 Recognizes Specifically IL-6−gp80 Complex

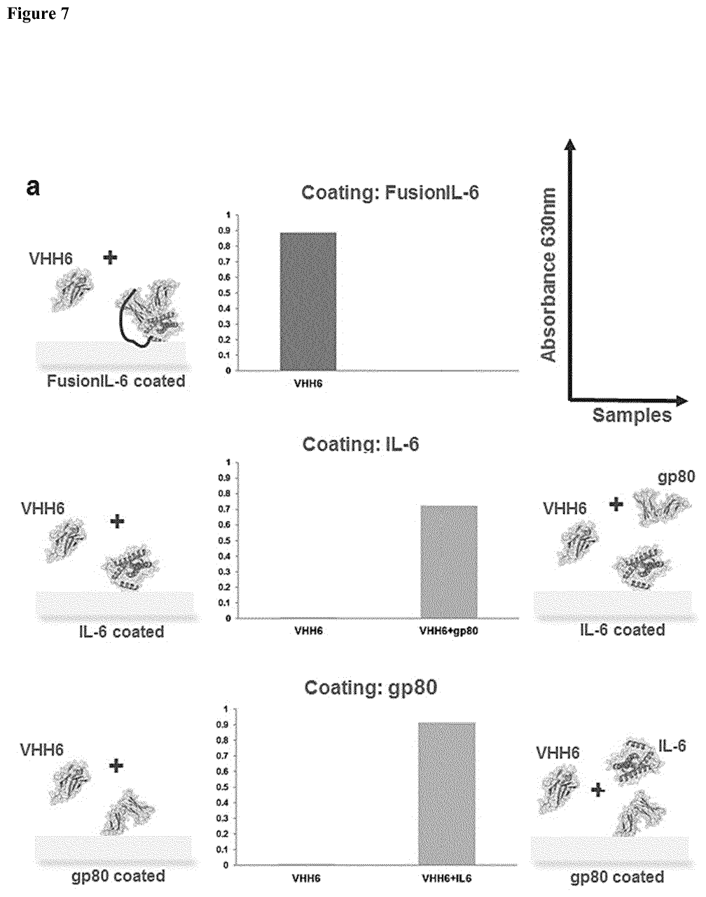

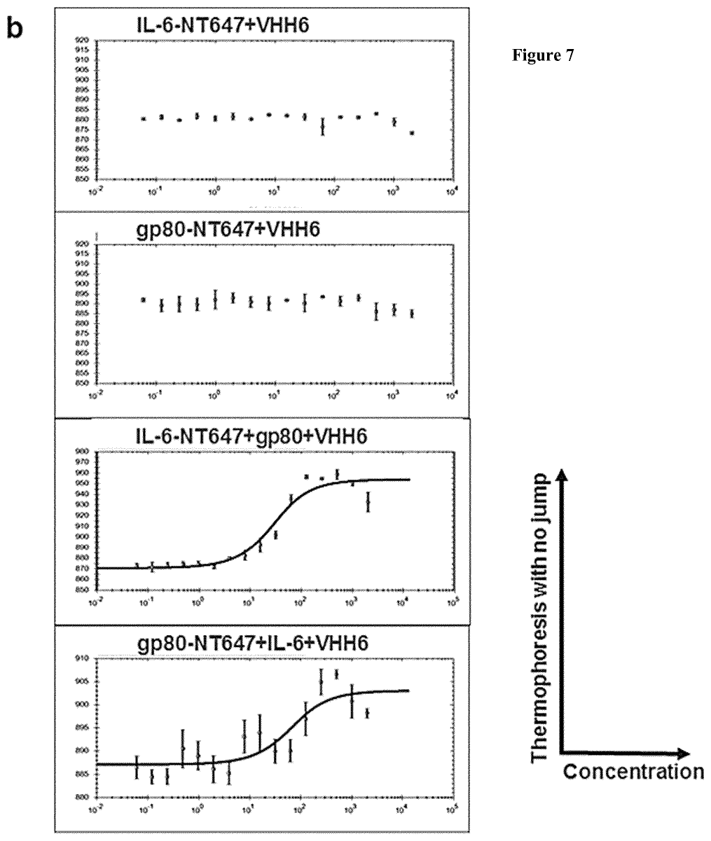

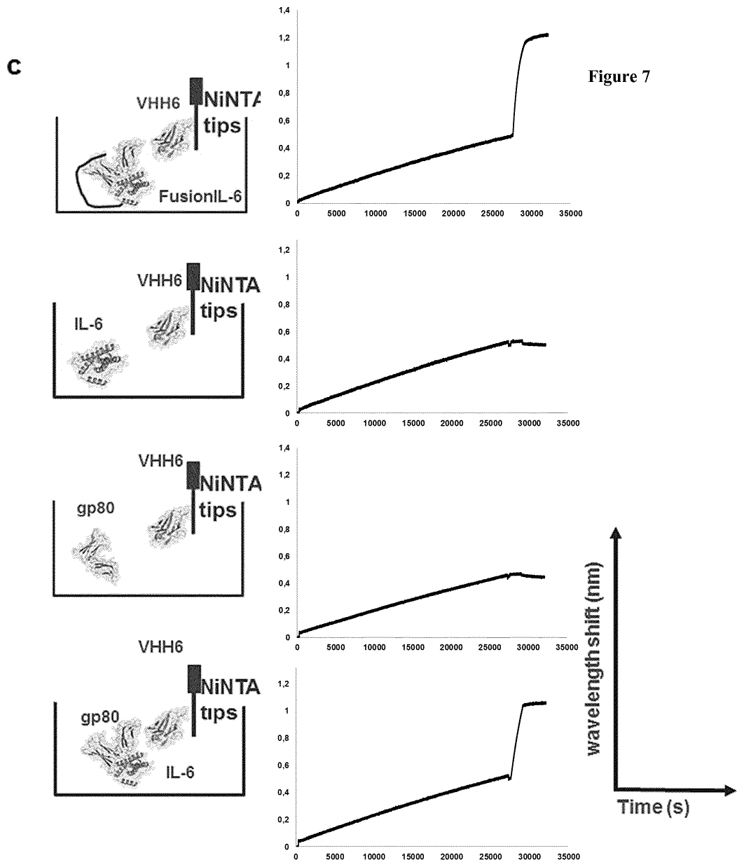

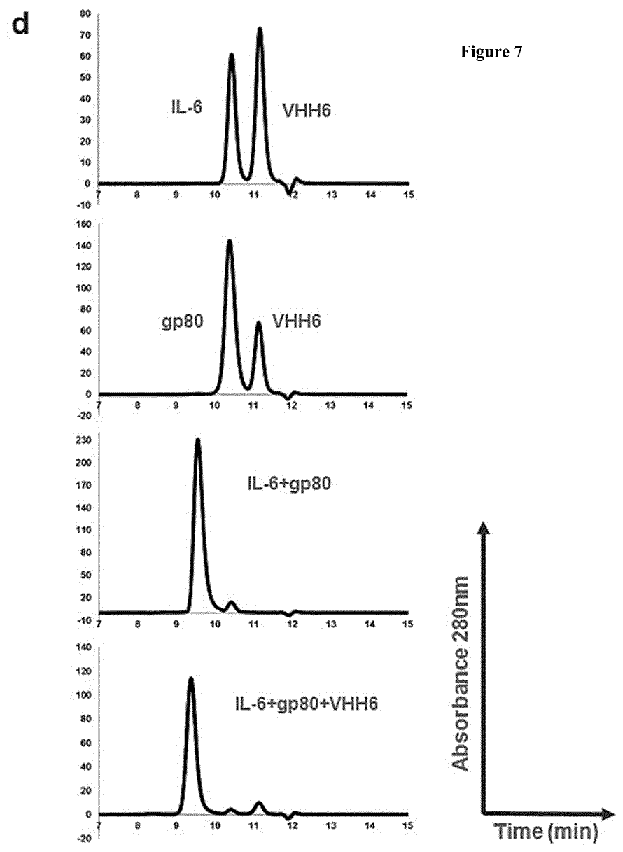

Immunization of camels with HyperIL-6, a fusion protein of gp80 joined to IL-6 with a flexible peptide linker and subsequent antibody discovery led to the generation of 41 unique VHH sequences. All 41 VHHs recognized HyperIL-6 and FusionIL-6 in an initial ELISA screen. FusionIL-6 was an in-house designed fusion protein between IL-6 and gp80. These 41 VHHs were separated according to their ability to bind to either IL-6 (30 VHHs) or gp80 (7 VHHs). Four VHHs recognized only FusionIL-6, but neither IL-6 nor gp80 alone. Of these, VHH6 was taken forward for further characterization, due to its superior yield in the transient expression system. Data generated using ELISA (FIG. 7a), MicroScale Thermophoresis (MST, FIG. 7b) and Bio-Layer Interferometry (Octet, FIG. 7c) and analytical size exclusion chromatography (FIG. 7d) showed that VHH6 is able to recognize and bind only to the IL-6−gp80 complex.

VHH6 Increases the Stability of the IL6-gp80 Complex

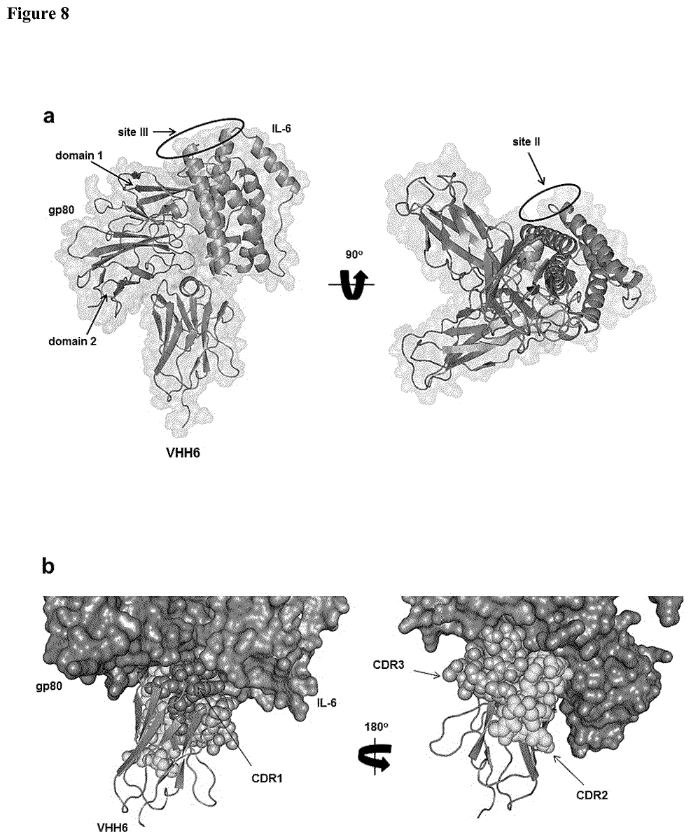

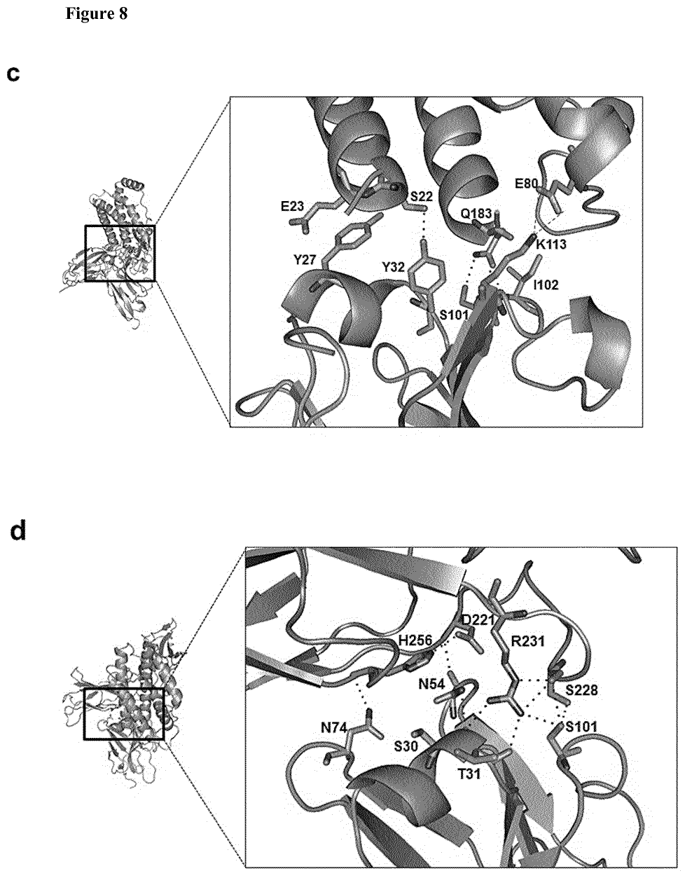

Four independent biophysical techniques strongly indicated that VHH6 specifically recognizes the complex between IL-6 and gp80. This suggested that VHH6 either recognized a conformational shift in one of the proteins or an epitope at the interface of IL-6-gp80 complex. To understand the nature of the epitope, the crystal structure of the VHH6−IL-6−gp80 complex was determined (see table 2 above). The complex crystallized in space group C 121 and was refined to 2.7 Å. The crystal structure confirmed that VHH6 binds to an epitope that spans across the interface of IL-6 and gp80. More specifically, it binds to the junction of IL-6 and domain I of gp80 with sites II and III on IL-6 remaining accessible for binding by gp130 (FIG. 8a). VHH6 binds to an epitope which is split spatially across IL-6 and gp80 almost evenly (45% of the epitope surface area lying on IL-6 and 55% on gp80).

All three of the complementarity-determining regions (CDRs) are involved in binding (FIG. 8b). Binding to IL-6 is mediated though multiple hydrogen bonds from the side chains of Tyr27, Tyr32, Ser101 and the backbone NH group of Ile102 (FIG. 8c). There is also a single salt bridge from Lys113 and a likely aliphatic interaction between Tyr27 and Glu23 of IL-6. Binding to gp80 is mediated through multiple hydrogen bonds from the backbone CO groups of Ser30, Thr31, Asn54 and the side chain of Asn74 (FIG. 8d).

To assess the contributions of the side chains to binding, single alanine substitutions at positions 27, 32, 74, 101 and 113, and double alanine substitutions at positions 27/32, 74/101 and 113/101 were generated. Either His-tagged VHH6, or mutant forms, were captured from supernatant on a nickel charged NTA chip and an equimolar mixture of IL-6 and gp80 injected over 300 s injection period. In a three-component system, it is not possible to calculate meaningful association rate constants. However, the dissociation rate constants, being concentration independent, were determined. For single residue mutants, the dissociation rates were very similar to VHH6 except for the Y27A mutant which showed a 1.9-fold increase. The dissociation rates for the double mutants all increased, with 3.8- and 4.3-fold increases observed for double mutants Asn74Ala/Ser101Ala and Ser101Ala/Lys113Ala respectively (Tables 3 and 4). These data confirmed the role in binding of the contacts identified in the crystal structure.

TABLE 3
Single mutants of VHH6

| | koff (1/s) |
|---|---|
| wtVHH6 | 3.6E−04 |
| N74A | 1.5E−04 |
| K113A | 2.6E−04 |
| Y32A | 3.0E−04 |
| S101A | 3.9E−04 |
| Y27A | 7.0E−04 |

TABLE 4
Double mutants of VHH6

| | koff (1/s) |
|---|---|
| wtVHH6 | 3.6E−04 |
| Y27A Y32A | 4.3E−04 |
| N74A S101A | 13.5E−04 |
| K113A S101A | 14.3E−04 |
| K113S S101A | 16.4E−04 |

To understand how VHH6 affects the structural dynamics of the IL-6−gp80 interaction, we used HDX-MS (Konermann, L., Pan, J. & Liu, Y. H. Hydrogen exchange mass spectrometry for studying protein structure and dynamics. Chemical Society reviews 40, 1224-1234 (2011)) (see FIG. 9a-b). A lower amount of deuterium exchange is indicative of decreased structural dynamics and/or protection of that region by a binding event, whereas higher exchange indicates an increase in dynamics. Data are obtained on the peptide level, whereby changes observed can be attributed to one or more of the residues contained therein. Lower deuterium uptake was seen in the presence of VHH6 for the sequences SSERIDKQ (SEQ ID NO: 11) and KDGCFQSCFNEE (SEQ ID NO: 12) (residues 21-28 and 70-81) in IL-6 and LSVTWQD (SEQ ID NO: 13) and MVKDLQHHAVIHD (SEQ ID NO: 14) (residues 215-221 and 250-262) in gp80, each of which contains residues located within the VHH6 interface, reinforcing that the epitope spans both proteins. No data were obtained for the residues 179-184 (RALRQM; SEQ ID NO: 15) at the C-terminus of IL6, also expected to interact directly with the VHH. Peptides in the regions MTTHLILRSFKEFLQSSL (SEQ ID NO: 16) (residues 161-178, IL-6) and PEGDSSFY (SEQ ID NO: 17) and RAQEEFGQGEWSE (SEQ ID NO: 18) (residues 162-169 and 274-286, gp80) also showed a decrease in HDX in the presence of the VHH (FIG. 9a, right); these are located at the IL6-gp80 interface, suggesting a tightening of the interaction here resulting in reduced structural dynamics. Finally, residues 126-144, LQKKAKNLAITTPDPTTN (SEQ ID NO: 19), in IL6 are shown to be less dynamic in the presence of the VHH6 (FIG. 9a, left); indeed, these are resolved in the crystal structure of the trimer whereas are too flexible to be modelled in the structure of IL6 alone (1ALU), indicating once more an increase in structural rigidity here upon VHH binding. There is also an indication of an allosteric conformational/dynamical change occurring on the distal side of IL6 (RKETCNKSNM, residues 40-49; SEQ ID NO: 20). For a more complete list of overlapping peptides defined by comparing HDX patterns see Tables 5 and 6.

TABLE 5
Peptides in IL-6 identified and quantified by HDX-MS. Black asterisk
(*) indicates deuterium uptake increasing the presence of VHH6; # indicates
deuterium uptake decreasing in the presence of VHH6.

| Start | End | | 2 timepoints | 4 timepoints | |
|---|---|---|---|---|---|
| Residue | Residue | Sequence | with p < 0.01 | with p < 0.01 | SEQ ID NO |
| 0 | 11 | APVPPGEDSKDV | | | 21 |
| 3 | 23 | PPGEDSKDVAAP | | | 22 |
| | | HRQPLTSSE | | | |
| 21 | 28 | SSERIDKQ | * | | 11 |
| 21 | 31 | SSERIDKQIRY | * | | 23 |
| 22 | 31 | SERIDKQIRY | * | | 24 |
| 24 | 33 | RIDKQIRYIL | * | * | 25 |
| 25 | 36 | IDKQIRYILDGI | * | * | 26 |
| 29 | 39 | IRYILDGISAL | | | 27 |
| 38 | 49 | ALRKETCNKSN | # | | 28 |
| | | M | | | |
| 39 | 49 | LRKETCNKSNM | # | | 29 |
| 40 | 49 | RKETCNKSNM | # | | 20 |
| 40 | 51 | RKETCNKSNMC | | | 30 |
| | | E | | | |
| 41 | 61 | KETCNKSNMCES | # | | 31 |
| | | SKEALAENN | | | |
| 42 | 56 | ETCNKSNMCESS | | | 32 |
| | | KEA | | | |
| 52 | 75 | SSKEALAENNLN | | | |
| | | LPKMAEKDGCF | | | 33 |
| | | Q | | | |
| 53 | 63 | SKEALAENNLN | | | 34 |
| 53 | 75 | SKEALAENNLNL | | | 35 |
| | | PKMAEKDGCFQ | | | |
| 55 | 65 | EALAENNLNLP | | | 36 |
| 58 | 67 | AENNLNLPKM | | | 37 |
| 58 | 74 | AENNLNLPKMA | * | * | 38 |
| | | EKDGCF | | | |
| 59 | 80 | ENNLNLPKMAE | * | | 39 |
| 60 | 67 | KDGCFQSGFNE | | | 40 |
| | | NNLNLPKM | | | |
| 60 | 70 | NNLNLPKMAEK | | | 41 |
| 60 | 82 | NNLNLPKMAEK | * | | 42 |
| 68 | 74 | DGCFQSGFNEET | * | * | 43 |
| | | AEKDGCF | | | |
| 70 | 81 | KDGCFQSGFNEE | * | | 44 |
| 75 | 84 | QSGFNEETCL | | | 45 |
| 85 | 92 | VKIITGLL | | | 46 |
| 85 | 93 | VKIITGLLE | | | 47 |
| 87 | 93 | IITGLLE | | | 48 |
| 98 | 108 | LEYLQNRFESS | | | 49 |
| 99 | 109 | EYLQNRFESSE | | | 50 |
| 99 | 110 | EYLQNRFESSEE | | | 51 |
| 99 | 112 | EYLQNRFESSEE | | | 52 |
| | | QA | | | |
| 99 | 120 | EYLQNRFESSEE | | | 53 |
| 100 | 110 | QARAVQMSTK | | | 54 |
| | | YLQNRFESSEE | | | |
| 100 | 112 | YLQNRFESSEEQ | | | 55 |
| | | A | | | |
| 104 | 114 | RFESSEEQARA | | | 56 |
| 106 | 112 | ESSEEQA | | | 57 |
| 106 | 116 | ESSEEQARAVQ | | | 58 |
| 109 | 116 | EEQARAVQ | | | 59 |
| 115 | 122 | VQMSTKVL | | | 60 |
| 115 | 122 | VQMSTKVL | | | 61 |
| 120 | 126 | KVLIQFL | | | 62 |
| 123 | 135 | IQFLQKKAKNLD | | | 63 |
| | | A | | | |
| 126 | 133 | LQKKAKNL | * | | 64 |
| 126 | 135 | LQKKAKNLDA | * | | 65 |
| 126 | 144 | LQKKAKNLDAIT | * | | 66 |
| | | TPDPTTN | | | |
| 126 | 145 | LQKKAKNLDAIT | | | 67 |
| | | TPDPTTNA | | | |
| 126 | 147 | LQKKAKNLDAIT | * | | 68 |
| | | TPDPTTNASL | | | |
| 129 | 143 | KAKNLDAITTPD | | | 69 |
| | | PTT | | | |
| 136 | 147 | ITTPDPTTNASL | * | * | 70 |
| 148 | 155 | LTKLQAQN | | | 71 |
| 148 | 155 | LTKLQAQN | | | 72 |
| 148 | 158 | LTKLQAQNQWL | | | 73 |
| 148 | 160 | LTKLQAQNQWL | | | 74 |
| | | QD | | | |
| 152 | 158 | QAQNQWL | | | 75 |
| 159 | 165 | QDMTTHL | * | | 76 |
| 159 | 167 | QDMTTHLIL | * | | 77 |
| 161 | 167 | MTTHLIL | * | | 78 |
| 166 | 172 | ILRSFKE | * | * | 79 |
| 166 | 173 | ILRSFKEF | * | * | 80 |
| 166 | 174 | ILRSFKEFL | * | | 81 |
| 168 | 174 | RSFKEFL | * | * | 82 |
| 168 | 178 | RSFKEFLQSSL | * | * | 83 |

TABLE 6
Peptides in gp80 identified and quantified by HDX-MS. Black
asterisk indicates deuterium uptake increasing the presence of VHH6.

| Start | End | | 2 timepoints | 4 timepoints | |
|---|---|---|---|---|---|
| Residue | Residue | Sequence | with p < 0.01 | with p < 0.01 | SEQ ID NO |
| 66 | 86 | HENLYFQGLAPRR | | | 84 |
| | | CPAQEVAR | | | |
| 73 | 82 | GLAPRRCPAQ | | | 85 |
| 73 | 83 | GLAPRRCPAQE | * | | 86 |
| 73 | 85 | GLAPRRCPAQEVA | * | | 87 |
| 78 | 96 | RCPAQEVARGAG | | | 88 |
| | | AGDVPPE | | | |
| 81 | 102 | AQEVARGAGAGD | * | | 89 |
| | | VPPEEPQLSC | | | |
| 82 | 95 | QEVARGAGAGDV | | | 90 |
| | | PP | | | |
| 83 | 94 | EVARGAGAGDVP | * | | 91 |
| 84 | 100 | VARGAGAGDVPP | * | | 92 |
| | | EEPQL | | | |
| 86 | 100 | RGAGAGDVPPEEP | * | | 93 |
| | | QL | | | |
| 92 | 110 | DVPPEEPQLSCFRK | * | | 94 |
| | | SPLSN | | | |
| 94 | 100 | PPEEPQL | | | 95 |
| 103 | 111 | FRKSPLSNV | | | 96 |
| 105 | 123 | KSPLSNVVCEWGP | | | 97 |
| | | RSTPSL | | | |
| 111 | 123 | VVCEWGPRSTPSL | | | 98 |
| 112 | 123 | VCEWGPRSTPSL | | | 99 |
| 115 | 129 | WGPRSTPSLTTKA | | | 100 |
| | | VL | | | |
| 124 | 130 | TTKAVLL | | | 101 |
| 130 | 141 | LVRKFQNSPAED | | | 102 |
| 131 | 141 | VRKFQNSPAED | | | 103 |
| 135 | 145 | QNSPAEDFQEP | | | 104 |
| 139 | 158 | AEDFQEPCQYSQE | | | 105 |
| | | SQKFSCQ | | | |
| 141 | 147 | DFQEPCQ | | | 106 |
| 146 | 164 | CQYSQESQKFSCQ | | | 107 |
| | | LAVPEG | | | |
| 147 | 164 | QYSQESQKFSCQL | | | 108 |
| | | AVPEG | | | |
| 148 | 158 | YSQESQKFSCQ | | | 109 |
| 152 | 158 | SQKFSCQ | | | 110 |
| 157 | 170 | CQLAVPEGDSSFYI | | | 111 |
| 159 | 168 | LAVPEGDSSF | * | | 112 |
| 162 | 168 | PEGDSSF | * | | 113 |
| 174 | 183 | CVASSVGSKF | | | 114 |
| 176 | 183 | ASSVGSKF | | | 115 |
| 195 | 205 | LQPDPPANITV | * | | 116 |
| 201 | 210 | ANITVTAVAR | | | 117 |
| 206 | 216 | TAVARNPRWLS | * | | 118 |
| 207 | 214 | AVARNPRW | | | 119 |
| 208 | 214 | VARNPRW | | | 120 |
| 208 | 217 | VARNPRWLSV | * | | 121 |
| 208 | 218 | VARNPRWLSVT | * | | 122 |
| 218 | 229 | TWQDPHSWNSSF | * | | 123 |
| 221 | 229 | DPHSWNSSF | | | 124 |
| 230 | 236 | YRLRFEL | | | 125 |
| 235 | 245 | ELRYRAERSKT | | | 126 |
| 237 | 245 | RYRAERSKT | | | 127 |
| 243 | 256 | SKTFTTWMVKDL | * | * | 128 |
| | | QH | | | |
| 250 | 262 | MVKDLQHHAVIH | * | | 14 |
| | | D | | | |
| 250 | 266 | MVKDLQHHAVIH | * | * | 129 |
| | | DAWSG | | | |
| 250 | 273 | MVKDLQHHAVIH | * | | 130 |
| | | DAWSGLRHVVQL | | | |
| 265 | 273 | SGLRHVVQL | | | 131 |
| 267 | 273 | LRHVVQL | | | 132 |
| 268 | 275 | RHVVQLRA | | | 133 |
| 274 | 283 | RAQEEFGQGE | * | * | 134 |
| 274 | 284 | RAQEEFGQGEW | * | * | 135 |
| 274 | 286 | RAQEEFGQGEWSE | * | * | 18 |
| 277 | 295 | EEFGQGEWSEWSP | | | 136 |
| | | EAMGTP | | | |
| 280 | 296 | GQGEWSEWSPEA | | | 137 |
| | | MGTPW | | | |
| 284 | 291 | WSEWSPEA | * | | 138 |
| 284 | 298 | WSEWSPEAMGTP | | | 139 |
| | | WTE | | | |
| 284 | 303 | WSEWSPEAMGTP | | | 140 |
| | | WTESRSPP | | | |
| 285 | 291 | SEWSPEA | | | 141 |
| 285 | 298 | SEWSPEAMGTPW | | | 142 |
| | | TE | | | |
| 285 | 303 | SEWSPEAMGTPW | | | 143 |
| | | TESRSPP | | | |
| 287 | 298 | WSPEAMGTPWTE | | | 144 |
| 289 | 303 | PEAMGTPWTESRS | | | 145 |
| | | PP | | | |
| 292 | 298 | MGTPWTE | | | 146 |

To investigate the effect of VHH6 on the binding kinetics of the IL-6−gp80 complex, a qualitative SPR experiment was performed, immobilizing IL-6 on a CMS chip and injecting gp80. As expected for a cytokine-receptor transient interaction, both fast association and dissociation phases were observed (FIG. 10a). Injection of VHH6 at the beginning of the dissociation phase of gp80, abrogated dissociation of gp80 from immobilized IL-6. VHH6 reduced the dissociation rate constant (k_{off}) for gp80 binding to IL-6 by 225-fold from 0.045 s⁻¹ to 0.0002 s⁻¹ (FIG. 10a). For brevity, FIGS. 10a and b only report the SPR sensorgrams for IL-6 immobilization. Similar results were obtained when gp80 or VHH6 were immobilized (Tables 7-10). These data demonstrate that not only does VHH6 bind to the IL-6−gp80 complex, but that it significantly impedes the dissociation of IL-6 from gp80.

TABLE 7

| IL-6 immobilised | k_{off} (1/s) | SE (k_{off}) | K_{D} (M) | SE (K_{D}) |
|---|---|---|---|---|
| gp80 | 0.046 | 5.40E−05 | 3.42E−08 | 7.20E−09 |
| gp80 + VHH6 (2 μM) | 1.94E−04 | 1.90E−07 | NA | |

TABLE 8

| gp80 immobilised | k_{off} (1/s) | SE (k_{off}) | K_{D} (M) | SE (K_{D}) |
|---|---|---|---|---|
| IL-6 | 0.048 | 4.70E−05 | 1.54E−08 | 1.50E−09 |
| IL-6 + VHH6 (2 μM) | 2.10E−04 | 2.80E−07 | NA | |

TABLE 9

| VHH6 immobilised | k_{off} (1/s) | SE (k_{off}) | K_{D} (M) |
|---|---|---|---|
| IL-6 + gp80 (2 μM) | 3.09E−04 | 4.40E−07 | NA |
| gp80 + IL-6 (2 μM) | 3.75E−04 | 6.00E−07 | NA |

TABLE 10

| gpl30 immobilised | kₒₙ (1/Ms) | k_{off} (1/s) |
|---|---|---|
| gp80 (IL-6 (2 μM)) | 3.6 10\hat{ }5 | 0.037 |
| gp80 (IL-6 (2 μM) VHH6 (2 μM)) | 4.7 10\hat{ }5 | 0.029 |
| IL-6 (gp80 (2 μM)) | 3.6 10\hat{ }5 | 0.035 |
| IL-6 (gp80 (2 μM) VHH6 (2 μM)) | 4.4 10\hat{ }5 | 0.036 |

The model created by superimposing the crystal structure solved for VHH6−IL-6−gp80 on the published data for the complex IL-6−gp80-gp130 (Boulanger, M. J., Chow, D. C., Brevnova, E. E. & Garcia, K. C. Hexameric structure and assembly of the interleukin-6/IL-6 alpha-receptor/gp130 complex. Science 300, 2101-2104 (2003)) (PDB 1P9M) indicated that VHH6 would not block the binding of IL-6−gp80 to gp130 (FIG. 14). To confirm this, gp130 extracellular domain was immobilized on the chip and a mixture containing IL-6-80 or VHH6−IL-6-80 was tested. As predicted, VHH6 did not impair the binding of the IL-6−gp80 complex to gp130 (FIG. 10c). Furthermore, stabilization by VHH6, did not affect the kinetics of IL-6−gp80 complex binding to gp130.

### VHH6 Modulates IL-6 Signaling in a Cell Model for Trans-Signaling

The next step in the characterization of VHH6 was to explore the cellular outcomes following the stabilization of IL-6−gp80 complex. Recruitment of gp130 by IL-6−gp80 leads to a series of phosphorylation events ultimately culminating in the phosphorylation of Signal Transducer and Activator of Transcription 3 (pSTAT3) molecules. Phosphorylated STAT3 molecules are translocated to the nuclei (Heinrich, P. C. et al. Principles of interleukin (IL)-6-type cytokine signalling and its regulation. The Biochemical journal 374, 1-20 (2003)), where they activate transcription of genes involved in the response to IL-6. IL-6-induced STAT3 phosphorylation peaks at 30 min after stimulation, and whilst largely exhausted within 60-90 min, a second wave is observed after 4 h (Wang, Y., van Boxel-Dezaire, A. H., Cheon, H., Yang, J. & Stark, G. R. STAT3 activation in response to IL-6 is prolonged by the binding of IL-6 receptor to EGF receptor. Proceedings of the National Academy of Sciences of the United States of America 110, 16975-16980 (2013)). It was postulated that adding VHH6 to IL-6 and gp80 mixture would increase the magnitude of STAT3 phosphorylation, thus mimicking the effects of HyperIL-6 in vitro.

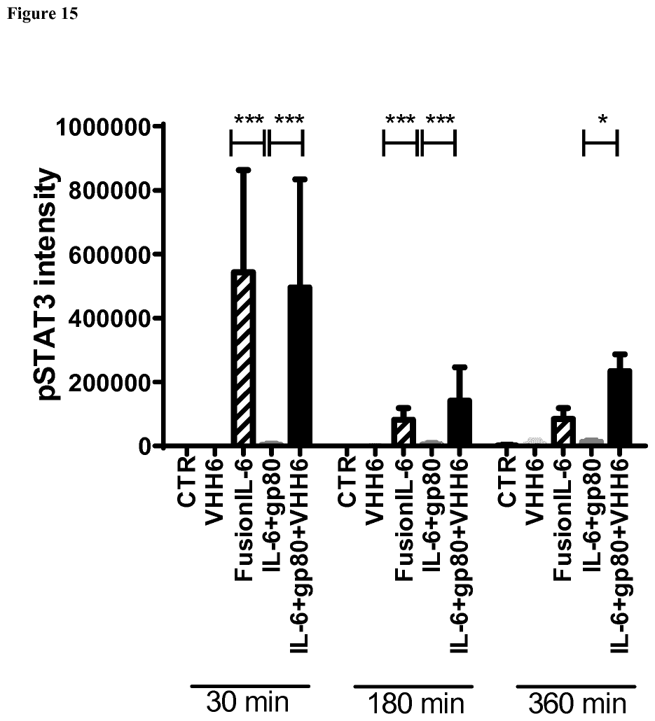

To have a controlled system, IL-6 and gp80 were added in a stoichiometric ratio 1:1 to Human Umbilical Vein Endothelial Cells (HUVEC), a well-established in vitro system for IL-6 trans-signaling (Romano, M. et al. Role of IL-6 and its soluble receptor in induction of chemokines and leukocyte recruitment. Immunity 6, 315-325 (1997)). HUVEC cells were treated with IL-6+gp80, VHH6+IL-6+gp80, and STAT3 phosphorylation was quantified at different time points using high-throughput fluorescence microscopy. FusionIL-6 was also included as internal control in all experiments (FIG. 15). As expected, VHH6+IL-6+gp80 showed a greater response than the mixture of IL-6 and gp80, with comparable intensity to FusionIL-6 at 30 min (FIG. 15), and sustained effects at later time points (FIG. 11). VHH6 addition to IL-6 and gp80 mixture clearly showed an increased activation of pSTAT3 at 30 min, with pSTAT3 signal predominantly detected in the nuclear compartment (FIG. 11a). Interestingly, treatment with VHH6 promoted a significant and sustained activation of pSTAT3 at later time points (FIG. 11b-c).

STAT3 phosphorylation is an indirect proof that the stabilization of the complex is instrumental in maximizing the effects of IL-6. Indeed, further proof would be to monitor the intracellular trafficking of IL-6 in the presence or absence of VHH6. It was investigated whether increased and sustained activation of STAT3 due to VHH6 was ascribable to an altered internalization of IL-6−gp80 using IL-6 labelled with NT647. FIG. 11d shows a representative time-course experiment with accumulation of IL-6-NT647 in vesicles at 30, 180 and 360 min. Also, accumulation of IL-6-NT647 co-localized with LAMP-1, indicating that IL-6NT647 has been taken up in vesicles (FIG. 11e). VHH6 promoted a significant increase in intracellular accumulation of IL-6-NT647 as early as 15 min and increasing over time when compared to IL-6−gp80 (FIG. 11f).

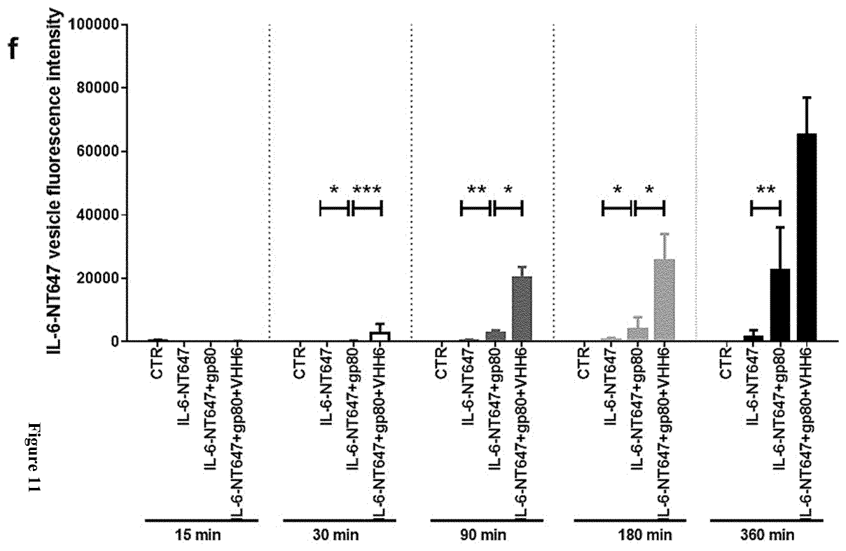

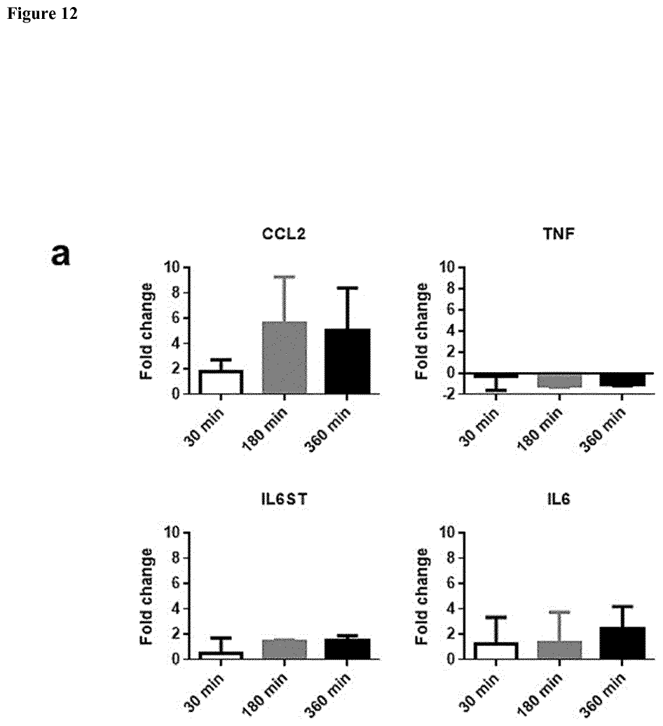

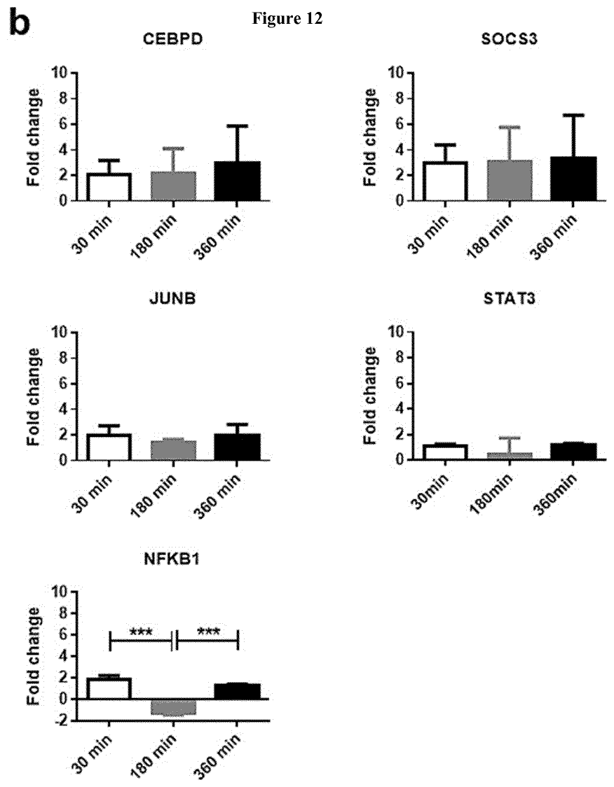

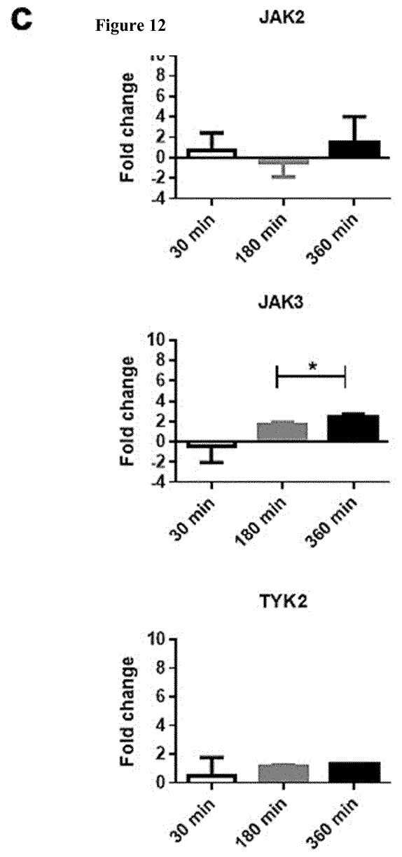

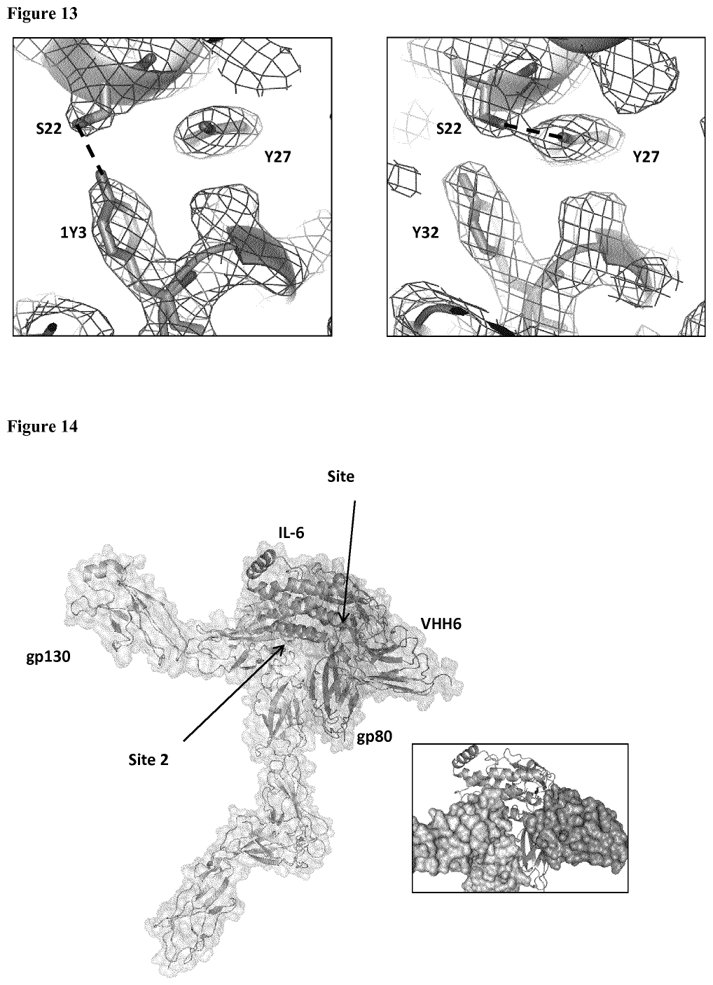

To further investigate the cellular effects of extended STAT3 phosphorylation and accumulation of IL-6 in intracellular vesicles, a transcriptomic profile of HUVEC cells was performed using a qPCR array specifically designed for the IL-6 pathway. The trans-signaling response to IL-6−gp80 treatment was compared in the absence (control group) and presence of VHH6 (treated samples). A heat-map generated from three replicates together clearly suggested that, despite the variability intrinsically related to primary cells, VHH6 treatment affected only a few genes related to cytokine/chemokines or transcription factors in IL-6 signaling (FIG. 16) and is related to the length of treatment. Those genes were grouped into three categories: chemokines/cytokines, transcription factors and signaling molecules (FIG. 12a-c). Of the chemokine and cytokine genes, CCL2, IL6 and IL6ST transcripts increased, whilst TNF decreased over time. Transcription factors genes for CEBPD and JUNB were upregulated at all times, whilst NFKB1 followed an up-down-up pattern. Signaling molecule SOCS3 was up-regulated at all times; STAT3 and JAK2 showed a wave-like distribution, whilst JAK3 and TYK2 increased over time. These results pointed towards an increased inflammatory response in HUVEC cells due to VHH6 treatment.

### Discussion

VHH6 is a prototype locking antibody which recognizes an epitope newly formed at the junction where IL-6 and gp80 interact to form a transient complex before recruiting the signaling receptor gp130. The methods described above blend several approaches to prove the junctional epitope nature of VHH6 and its ability to stabilize the complex between IL-6 and gp80 by locking together the two proteins.

HDX-MS revealed how VHH6 alters the dynamic or “breathability” which characterizes the structure of the IL-6−gp80 complex. X-ray crystallography confirmed that VHH6 clamps the two molecules together without interfering with the recruitment of gp130 into the signaling complex. Once the VHH6−IL-6−gp80 ternary complex is formed, IL-6 and gp80 are no longer able to dissociate, as shown by SPR experiments.

The alteration of the kinetics between IL-6 and gp80 has allowed the structure of the IL-6−gp80 complex to be solved in the absence of gp130. VHH6 engages simultaneously two proteins using its CDRs. VHH6 differs from bispecific antibodies, specifically engineered to target two proteins simultaneously. In the literature there are several examples of junctional antibodies. Dixin et al (Diskin, R., Marcovecchio, P. M. & Bjorkman, P. J. Structure of a clade C HIV-1 gp120 bound to CD4 and CD4-induced antibody reveals anti-CD4 polyreactivity. Nature structural & molecular biology 17, 608-613 (2010)), described a bimolecular epitope scFv where one CDR of the V_{H} domain contacts CD4 and one CDR of the V_{L} domain contacts gp120. The crystal structure of a neutralizing antibody able to stabilize hemagglutinin stem was recently described by Impagliazzo et al (Impagliazzo, A., et al. A stable trimeric influenza hemagglutinin stem as a broadly protective immunogen. Science 349(6254), 1301-1306 (2015)), while an example of an antibody (KZ52) that engages three discontinuous segments of Ebola virus GP1 and GP2 proteins was published by Lee et al (Lee, J. E., et al. Structure of the Ebola virus glycoprotein bound to an antibody from a human survivor. Nature. 454(7201), 177-82 (2008)).

However, VHH6 is the prototype of an antibody which stabilizes the transient PPI between IL-6 and gp80. This is a perfect example of transient PPIs, characterized by fast association and dissociation rates, which has an important consequence from an immunological point of view to control the downstream signaling of e.g. pro-inflammatory cytokines. The key feature is that, as VHH6 binds to the complex IL-6−gp80, it effectively acts as an agonistic antibody for IL-6, as proved by the in-depth characterization ranging from crystal structure to binding properties to biological activity.

VHH6 works similarly to an entropic trap, making collisions between IL-6 and gp80 efficacious while increasing the concentration at equilibrium of the IL-6−gp80 complex. The hypothesis is that VHH6, by overcoming the entropic cost required by the system to keep IL-6 and gp80 together, allows the stable complex formed to recruit gp130 and form the hexameric signaling complex.

The ability of VHH6 to alter the interaction between IL-6 and gp80 at the junction of the two molecules, without interfering with the recruitment of the signaling partner gp130, made it possible to reproduce in vitro some of the features of chronic inflammation using HUVEC cells.

In the HUVEC cell model for IL-6 trans-signaling, the addition to VHH6 to IL-6 and gp80 simply translates into a faster uptake and greater intracellular accumulation of the IL-6 in lysosomal vesicles. Not only did VHH6 significantly increase the pSTAT3 signal within 30 min of treatment, but the effect extended to 360 min at a significantly greater level than that achieved with the mixture of just IL-6 and gp80.

Sustained STAT3 phosphorylation is associated with auto-inflammatory disease, such as rheumatoid arthritis (RA) (Yu, H., Pardoll, D. & Jove, R. STATs in cancer inflammation and immunity: a leading role for STAT3. Nature reviews. Cancer 9, 798-809 (2009)). To confirm that stabilization of IL-6−gp80 leads in vitro to a pro-inflammatory response, the transcriptomic profile of HUVEC cells treated with VHH6−IL-6−gp80 was compared to that of cells treated with IL-6−gp80. The up-regulation of genes involved in the pro-inflammatory response was directly related to the length of treatment with VHH6−IL-6-gp80. Further supporting the findings that pSTAT3 was readily translocated into the nuclei, the up-regulated genes were linked to transcription factors and cytokine activity regulators (NFKB1, CEBPD, JUNB, STAT3, SOCS3), kinases of the JAK family, cytokine and cytokine receptors (IL-6 and IL-6ST, alias gp130) and chemokines (CCL2, alias monocyte chemotactic protein 1 or MCP1). For the relevance to disease and disease biomarkers, the most interesting of these genes is the one coding for MCP1. MCP1 is upregulated in synovia (Koch, A. E. et al. Enhanced production of monocyte chemoattractant protein-1 in rheumatoid arthritis. The Journal of clinical investigation 90, 772-779 (1992)) and in serum samples (Hueber, W. et al. Proteomic analysis of secreted proteins in early rheumatoid arthritis: anti-citrulline autoreactivity is associated with up regulation of proinflammatory cytokines. Annals of the rheumatic diseases 66, 712-719 (2007)) of RA patients. It has been shown that pro-inflammatory stimulation of HUVEC cells leads to the production of IL-6 and CCL2, with both contributing to an increase in endothelial permeability (Stamatovic, S. M., Keep, R. F., Kunkel, S. L. & Andjelkovic, A. V. Potential role of MCP-1 in endothelial cell tight junction ‘opening’: signaling via Rho and Rho kinase. Journal of cell science 116, 4615-4628 (2003) and Desai, T. R., Leeper, N. J., Hynes, K. L. & Gewertz, B. L. Interleukin-6 causes endothelial barrier dysfunction via the protein kinase C pathway. The Journal of surgical research 104, 118-123 (2002)). Therefore, the manipulation of STAT3 signaling using a junctional epitope antibody appears to reflect the modulation that can occur in a disease state.

VHH6 offers great potential in future studies. Due to its ability to stabilize and modulate the IL-6 and gp80 interaction, VHH6 could find an application in understanding the role played by IL-6 cross-talk with other cytokines or to verify the hypothesis that the switch between IL-6 (pro-inflammatory cytokine) and anti-inflammatory cytokine (e.g. IL-10), which share STAT3 as a transcription factor, might be due to an extended pSTAT3 signal (Braun, D. A., Fribourg, M. & Sealfon, S. C. Cytokine response is determined by duration of receptor and signal transducers and activators of transcription 3 (STAT3) activation. The Journal of biological chemistry 288, 2986-2993 (2013)). The therapeutic applications of VHH6 or a fusion protein VHH6-gp80 in biological systems could be explored where IL-6 plays a role as a growth factor (Galun, E. & Rose-John, S. The regenerative activity of interleukin-6. Methods in molecular biology 982, 59-77 (2013)) e.g. after injury in liver regeneration (Wuestefeld, T. et al. Interleukin-6/glycoprotein 130-dependent pathways are protective during liver regeneration. The Journal of biological chemistry 278, 11281-11288 (2003)) or activation of hepatocyte-like progenitor cells (Best, D. H., Butz, G. M. & Coleman, W. B. Cytokine-dependent activation of small hepatocyte-like progenitor cells in retrorsine-induced rat liver injury. Experimental and molecular pathology 88, 7-14 (2010)) and in expansion of hematopoietic progenitor cells (Fischer, M. et al. I. A bioactive designer cytokine for human hematopoietic progenitor cell expansion. Nature biotechnology 15, 142-145 (1997)). Lastly and particularly pertinent for drug discovery purposes, VHH6, by locking IL-6 and gp80 together, could be instrumental for screening more specific inhibitors (peptides, small molecules or even antibodies) of the IL-6−gp80 complex thus leading to a selective inhibition of IL-6 trans-signaling pathway in pathologies.

In conclusion, the data generated in this proof-of-concept study demonstrates the functional consequences of manipulating the interface between two proteins by stabilizing their interaction with a complex-specific antibody. Using antibodies, instead of fusion protein constructs, offers the advantage of proteins with well-defined chemical-physical characteristics and the capability of preserving the correct conformation of the complex as a functional unit. Junctional epitope antibodies like VHH6, which lock together and stabilize a transient PPI, would be an excellent tool to explore novel biological hypothesis. Ultimately this may lead to a different way of approaching drug discovery and therapeutic interventions, when the target is not a single molecule but a multiprotein complex.

### Example 2—Use of a Junctional Binding Protein in Cross-Linking Antibody Fusions and their Antigens

This Example illustrates how use of a junctional binding protein to cross-link two antibody fusions and thereby their associated antigens may be tested. This is based on assays described in WO 2015/181282 (see for example Examples 8/9/10). WO 2015/181282 provides an example of general bispecific targeting which only provides a function when both markers are targeted. WO 2016/009030 and WO 2016/009029 present more specific examples, where CD22/CD79 and CD45/CD79 are targeted.

### General Method 1

Human PBMC derived from platelet apheresis cones are banked as frozen aliquots. Prior to an assay being performed, cells are thawed, washed in DMEM (Life Technologies) and allowed to acclimatise to a 37° C. and 5% CO₂ environment.

### General Method 2

Fab′A-Component A (inactive-IL-6) and Fab′B-Component B (inactive GP80) are incubated together for 90 minutes (in a 37° C./5% CO₂ environment) either in the presence of absence of the junctional antibody VHH6 before mixing with 2.5×10⁵ PBMC in V-bottomed 96 well plates. PBMC plus the cross-linked (Fab′A-Component A and Fab′B-Component B and VHH6) or non-cross-linked (Fab′A-Component A and Fab′B-Component B) combinations are then incubated together for a further 90 minutes. After this time B cells are activated by the addition of 200 nM of goat F(ab′)2 anti-human IgM (Southern Biotechnology) for 8 minutes at 37° C. The signalling reaction is then halted by adding an equal volume of Cytofix buffer (BD Biosciences). Plates are then left at room temperature for 15 minutes before centrifugation at 500 g for 5 minutes. Excess supernatant is discarded from the cell pellet which is resuspended in flow buffer and washed once more. Cells are then resuspended in ice cold Perm Buffer III (BD Biosciences) for 30 minutes before being washed twice in flow buffer.

### General Method 3

Cells are activated as described in general method 2 and stained with a fluorescently labelled anti-CD20 antibody (BD Biosciences), anti-phospho Akt antibody that recognises a modified serine residue at position 473, an anti-phospho PLCg2 antibody that recognises a modified tyrosine residue at position 759 and an anti-IκB antibody that recognises total 1 KB. Plates are then resuspended and incubated for 1 hour at room temperature in the dark. After this time plates were washed a further two times and resuspended in 25 μl of flow buffer. Cellular expression of CD20, Akt and PLCg2 is measured using an Intellicyt HTFC™ flow cytometer

### Example 3—in Silico Generation of Humanized Sequences of the VHH6 Antibody

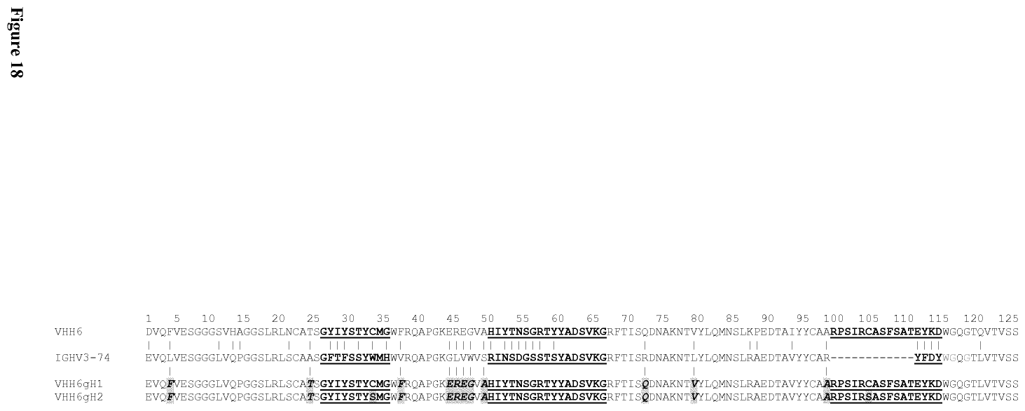

Llama VHH6 was humanized by grafting the CDRs from the Llama VHH-region onto a human germline antibody V_{H}-region framework. A number of framework residues from the Llama VHH-region were also retained in the humanized sequence. These residues were selected using the protocol outlined by Adair et al. (Humanised antibodies. WO91/09967). Alignments of the Llama VHH (donor) V-region sequence with the human germline (acceptor) V-region sequence are shown in FIG. 18, together with the designed humanized sequences.

The CDRs grafted from the donor to the acceptor sequence are as defined by Kabat et al. (Kabat et al., Sequences of proteins of immunological interest, 4^{th} ed, pp. vii-804 (1987)), with the exception of CDR-H1 where the combined Chothia/Kabat definition is used (see Adair et al., 1991 Humanised antibodies. WO91/09967). Human V-region IGHV3-74 plus IGHJH4 J-region (http://www.imgt.org/) was chosen as the acceptor for the VHH CDRs. The heavy chain framework residues in graft gH1 are all from the human germline gene, with the exception of residues 4, 24, 37, 44, 45, 46, 47, 49, 71, 78 and 94 (Kabat numbering), where the donor residues Phenylalanine (F4), Threonine (T24), Phenylalanine (F37), Glutamic acid (E44), Arginine (R45), Glutamic acid (E46), Glycine (G47), Alanine (A49), Glutamine (Q71), Valine (V78) and Alanine (A94) were retained, respectively.

Informal Sequence Listing

SEQ ID NO: 1 (VHH6 heavy chain variable region)
DVQFVESGGGSVHAGGSLRLNCATSGYIYSTYCMGWFRQAPGKEREGVAHIYTNSGRTYYA
DSVKGRFTISQDNAKNTVYLQMNSLKPEDTAIYYCAARPSIRCASFSATEYKDWGQGTQVT
VSS
SEQ ID NO: 2 (VHH6 CDR1)
GYIYSTYCMG
SEQ ID NO: 3 (VHH6 CDR2)
HIYTNSGRTYYADSVKG
SEQ ID NO: 4 (VHH6 CDR3)
RPSIRCASFSATEYKD
SEQ ID NO: 5 (VHH6 heavy chain variable region)
GATGTGCAGTTCGTGGAGTCTGGGGGAGGCTCGGTGCACGCTGGAGGGTCTCTAAGACTCA
ACTGTGCTACCTCTGGATACATTTACAGTACCTATTGCATGGGATGGTTCCGCCAGGCTCC
AGGGAAGGAGCGTGAGGGGGTCGCACATATTTATACGAATAGTGGACGCACATACTATGCC
GACTCCGTGAAGGGCCGATTCACCATCTCCCAAGACAACGCCAAGAACACGGTGTATCTGC
AAATGAACAGCCTGAAACCTGAGGACACGGCCATCTATTACTGTGCGGCCCGTCCCAGTAT
TAGGTGTGCTTCCTTCAGCGCCACCGAATATAAGGACTGGGGCCAGGGGACCCAGGTCACC
GTCTCCTCG
SEQ ID NO: 6 (VHH6 CDR1)
GGATACATTTACAGTACCTATTGCATGGGA
SEQ ID NO: 7 (VHH6 CDR2)
CATATTTATACGAATAGTGGACGCACATACTATGCCGACTCCGTGAAGGGC
SEQ ID NO: 8 (VHH6 CDR3)
CGTCCCAGTATTAGGTGTGCTTCCTTCAGCGCCACCGAATATAAGGAC
SEQ ID NO: 9 (IL-6 from FIG. 9)
GSAPVPPGED SKDVAAPHRQ PLTSSERIDK QIRYILDGIS ALRKETCNKS
NMCESSKEAL AENNLNLPKM AEKDGCFQSG FNEETCLVKI ITGLLEFEVY
LEYLQNRFES SEEQARAVQM STKVLIQFLQ KKAKNLDAIT TPDPTTNASL
LTKLQAQNQW LQDMTTHLIL RSFKEFLQSS LRALRQM
SEQ ID NO: 10 (gp80 from FIG. 9)
GLAP RRCPAQEVAR GAGAGDVPPE EPQLSCFRKS PLSNVVCEWG
PRSTPSLTTK AVLLVRKFQN SPAEDFQEPC QYSQESQKFS CQLAVPEGDS
SFYIVSMCVA SSVGSKFSKT QTFQGAGILQ PDPPANITVT AVARNPRWLS
VTWQDPHSWN SSFYRLRFEL RYRAERSKTF TTWMVKDLQH HAVIHDAWSG
LRHVVQLRAQ EEFGQGEWSE WSPEAMGTPW TESRSPP
SEQ ID NOs: 11-147
Sequences shown in the Examples
SEQ ID NO: 147 (VHH6gH1 heavy chain variable region)
EVQFVESGGGLVQPGGSLRLSCATSGYIYSTYCMGWFRQAPGKEREGVAHIYTNSGRTYYA
DSVKGRFTISQDNAKNTVYLQMNSLRAEDTAVYYCAARPSIRCASFSATEYKDWGQGTLVT
VSS
SEQ ID NO: 148 (VHH6gH2 heavy chain variable region)
EVQFVESGGGLVQPGGSLRLSCATSGYIYSTYSMGWFRQAPGKEREGVAHIYTNSGRTYYA
DSVKGRFTISQDNAKNTVYLQMNSLRAEDTAVYYCAARPSIRSASFSATEYKDWGQGTLVT
VSS
SEQ ID NO: 149 (VHH6gH1 CDR1)
GYIYSTYCMG
SEQ ID NO: 150 (VHH6gH2 CDR1)
GYIYSTYSMG
SEQ ID NO: 151 (VHH6gH1 and VHH6gH2 CDR2)
HIYTNSGRTYYADSVKG
SEQ ID NO: 152 (VHH6gH1 CDR3)
RPSIRCASFSATEYKD
SEQ ID NO: 153 (VHH6gH2 CDR3)
RPSIRSASFSATEYKD
SEQ ID NO: 154 (Human IGHV3-74 IGHJH4 acceptor framework)
EVQLVESGGGLVQPGGSLRLSCAASGFTFSSYWMHWVRQAPGKGLVWVSRINSDGSSTSYA
DSVKGRFTISRDNAKNTLYLQMNSLRAEDTAVYYCARYFDYWGQGTLVTVSS

1. A binding protein specific for a junctional epitope created by a transient protein:protein interaction, which binding protein stabilises the protein:protein interaction.

2. The binding protein of claim 1, which:
(a) has no detectable binding to each individual component of the protein:protein interaction in the absence of the other; and/or
(b) has a dissociation constant (K_{D}) for binding to the protein:protein interaction that is at least 10 fold lower than the K_{D} for binding to each individual protein component of the protein:protein interaction.

3. The binding protein of claim 1 or 2, wherein between 20% and 80% of the epitope is present on one of the proteins of the protein:protein interaction, with the remainder of the epitope present on the other protein.

4. The binding protein of claim 1, 2 or 3, wherein the two proteins of the transient protein:protein interaction have a dissociation rate (k_{off}) in the absence of the binding protein of greater than 0.01 s⁻¹, optionally greater than 0.025 s⁻¹.

5. The binding protein of any one of the preceding claims, wherein in the presence of the binding protein the two components of the transient protein:protein interaction have a dissociation rate (k_{off}) of less than 0.01 s⁻¹, optionally less than 0.001 s⁻¹ or less than 0.0005 s⁻¹.

6. The binding protein of claim 4 or 5, wherein the k_{off} is measured by surface plasmon resonance at 25° C.

7. The binding protein of any one of the preceding claims, wherein the dissociation rate (k_{off}) for the transient protein:protein interaction is reduced at least 10 fold, at least 50 fold or at least 100 fold in the presence of the binding protein.

8. The binding protein of any one of the preceding claims, wherein the dissociation constant (K_{D}) for the protein:protein interaction is reduced at least 10 fold, at least 50 fold or at least 100 fold in the presence of the binding protein.

9. The binding protein of any of the preceding claims, wherein the protein:protein interaction is the IL-6/gp80 interaction.

10. The binding protein of any one of the preceding claims, which is an antibody or fragment thereof.

11. The binding protein of claim 10, wherein the heavy chain variable region (V_{H}) of the antibody or fragment thereof has interactions with both proteins of the protein:protein interaction, the light chain variable region (V_{L}) has interactions with both proteins of the protein:protein interaction, or both the V_{H} and V_{L} have interactions with both proteins of the protein:protein interaction.

12. The binding protein of claim 10 or 11, wherein one or more individual complementarity determining regions (CDRs) of the V_{H} and/or V_{L} interact with both components of the protein:protein interaction.

13. The binding protein of any one of the preceding claim 10, 11 or 12, which is a Fab, scFv or VHH.

14. The binding protein of any one of claims 10-13, wherein the protein:protein interaction is the IL-6/gp80 interaction and wherein the antibody or fragment thereof comprises at least one heavy chain complementarity determining region (HCDR) sequence selected from SEQ ID NOs: 2-4.

15. The binding protein of claim 14, wherein the antibody or fragment thereof comprises a HCDR3 of SEQ ID NO: 4.

16. The binding protein of claim 15, which additionally comprises a HCDR1 of SEQ ID NO: 2 and a HCDR2: of SEQ ID NO: 3.

17. The binding protein of any one of claims 10-13, wherein the protein:protein interaction is the IL-6/gp80 interaction and wherein the antibody or fragment thereof comprises HCDR1, HCDR2 and HCDR3 sequences contained within a heavy chain variable region (V_{H}) of SEQ ID NO: 1.

18. The binding protein of any one of claims 10-17, wherein the antibody or fragment thereof comprises a V_{H} sequence of SEQ ID NO: 1, or a sequence at least 90% identical thereto.

19. The binding protein of claim 18, wherein the HCDR1, HCDR2 and HCDR3 sequences consist of SEQ ID NOs: 2, 3 and 4 respectively, and wherein the remainder of the V_{H} has at least 90% identity to SEQ ID NO: 1.

20. The binding protein of any one of claims 10-13, wherein the binding protein is an antibody or fragment thereof which binds to the same epitope on IL-6/gp80 as, or competes for binding with, an antibody or fragment thereof as defined in any one of claims 14-19.

21. An isolated polynucleotide encoding one or both chains of an antibody or fragment thereof as defined in any one of claims 10-20.

22. The isolated polynucleotide of claim 21, which encodes a V_{H} of SEQ ID NO: 1 and has a sequence of SEQ ID NO: 5.

23. A vector comprising the polynucleotide of claim 21 or 22.

24. A host cell comprising the vector of claim 23.

25. A method of producing an antibody or fragment thereof as defined in any one of claims 10-20, said method comprising culturing the host cell as defined in claim 24 in a medium to produce the antibody according to any one of claims 10-20 and collecting the antibody or fragment thereof from the culture.

26. A pharmaceutical composition comprising the binding protein of any one of claims 1-20 and a pharmaceutically acceptable carrier or diluent.

27. Use of a binding protein as defined in any one of claims 1-20 in delivering cargo to a target cell.

28. The use of claim 27, wherein the cargo is a diagnostic or therapeutic agent.

29. A method of delivering cargo to a target cell, comprising:
(1) administering two targeting molecules, wherein:
the first targeting molecule comprises a first targeting portion which binds specifically to a first target molecule on the target cell and wherein the targeting portion is linked to the first protein component of a transient protein:protein interaction; and
the second targeting molecule comprises a second targeting portion which binds specifically to a second target molecule on the target cell and wherein the targeting portion is linked to the second protein component of the transient protein:protein interaction;
(2) administering a binding protein specific for a junctional epitope created by the transient protein:protein interaction, which binding protein is linked to the cargo and wherein the binding protein is as defined in any one of claims 1-20 above.

30. The method of claim 29, wherein the cargo is a diagnostic or therapeutic agent.

31. The method of claim 29 or 30, wherein the target cell is a cancer cell.

32. The method of claim 29, 30 or 31, wherein the first and second targeting portions are antibodies or fragments thereof, or ligands for the target molecules.

33. The method of any one of claims 29-32, wherein the first and second target molecules are antigens or receptors.

34. Use of a binding protein as defined in any one of claims 1-20 in switching on a biological effect.

35. A method of switching on a biological effect, comprising:
(1) administering two targeting molecules, wherein:
the first targeting molecule comprises a first targeting portion which binds specifically to a first target molecule on the target cell and wherein the targeting portion is linked to the first protein component of a transient protein:protein interaction; and
the second targeting molecule comprises a second targeting portion which binds specifically to a second target molecule on the target cell and wherein the targeting portion is linked to the second protein component of the transient protein:protein interaction; and
(2) administering a binding protein specific for a junctional epitope created by the transient protein:protein interaction, wherein the binding protein is as defined in any one of claims 1-20 and wherein administration of the binding protein switches on the biological effect.

36. A method of altering of a biological effect, comprising:
(1) administering two targeting molecules, wherein:
the first targeting molecule comprises a first targeting portion which binds specifically to a first target molecule on the target cell and wherein the targeting portion is linked to the first protein component of a transient protein:protein interaction; and
the second targeting molecule comprises a second targeting portion which binds specifically to a second target molecule on the target cell and wherein the targeting portion is linked to the second protein component of the transient protein:protein interaction; and
(2) administering a binding protein specific for a junctional epitope created by the transient protein:protein interaction, wherein the binding protein is as defined in any one of claims 1-20 and wherein administration of the binding protein alters the biological effect.

37. The method of claim 35 or 36, wherein the first and second targeting portions are antibodies or fragments thereof, or ligands for the target molecules.

38. The method of any one of claims 35-37, wherein the first and second target molecules are antigens or receptors.

39. Use of a binding protein as defined in any one of claims 1-20 in cross-linking target cells.

40. A method of cross-linking two target cells using a binding protein, wherein the first target cell expresses a first target molecule and the second target cell expresses a second target molecule, and wherein the method comprises:
(1) administering two targeting molecules, wherein:
the first targeting molecule comprises a first targeting portion which binds specifically to the first target molecule on the first target cell and wherein the targeting portion is linked to the first protein component of a transient protein:protein interaction; and
the second targeting molecule comprises a second targeting portion which binds specifically to the second target molecule on the second target cell and wherein the targeting portion is linked to the second protein component of the transient protein:protein interaction; and
(2) administering a binding protein specific for a junctional epitope created by the transient protein:protein interaction, wherein the binding protein is as defined in any one of claims 1-20.

41. A method of cross-linking two target cells, wherein the first target cell expresses a first and second target molecule and the second target cell expresses a third target molecule, and wherein the method comprises:
(1) administering two targeting molecules, wherein:
the first targeting molecule comprises a first targeting portion which binds specifically to the first target molecule on the first target cell and wherein the targeting portion is linked to the first protein component of a transient protein:protein interaction; and
the second targeting molecule comprises a second targeting portion which binds specifically to the second target molecule on the first target cell and wherein the targeting portion is linked to the second protein component of the transient protein:protein interaction; and
(2) administering a third targeting molecule comprising a third targeting portion which binds specifically to the third target molecule on the second target cell, wherein the targeting portion is linked to a binding protein specific for a junctional epitope created by the transient protein:protein interaction and wherein the binding protein is as defined in any one of claims 1-20.

42. A method of cross-linking two target cells, wherein the first target cell expresses a first and second target molecule and wherein the second target cell is engineered to present a binding protein on its surface, and wherein the method comprises administering two targeting molecules, where:
the first targeting molecule comprises a first targeting portion which binds specifically to the first target molecule on the first target cell and wherein the targeting portion is linked to the first protein component of a transient protein:protein interaction; and
the second targeting molecule comprises a second targeting portion which binds specifically to the second target molecule on the first target cell and wherein the targeting portion is linked to the second protein component of the transient protein:protein interaction;
wherein the binding protein is as defined in any one of claims 1-20 above.

43. The method of claim 42, wherein the second target cell is engineered to express the binding protein as a cell membrane fusion protein.

44. The method of any one of claims 40-43, wherein:
(a) the first, second and/or third, if present, targeting portions are antibodies or fragments thereof, or ligands for the target molecules; and/or
(b) the first, second and/or third, if present, target molecules are antigens or receptors.

45. The method of any one of claims 40-44, wherein the first or second target cell is an effector cell, such as a T cell.

46. The method of any one of claims 40-45, wherein the first or second target cell is a cancer cell.

47. A method of treatment of the human or animal body, comprising administering a binding protein according to any one of claims 1-20.

48. A method of tissue regeneration, comprising administering a binding protein as defined in claim 9 or any one of claims 14-20.

49. The method according to claim 48, which is a method of liver regeneration.

50. A method of expanding hematopoietic progenitor cells or hepatocyte-like progenitor cells using a binding protein as defined in claim 9 or in any one of claims 14-20.

51. A method of producing an antibody specific for a junctional epitope created by a transient protein:protein interaction, said method comprising immunising an animal with a fusion protein of the stabilised transient protein:protein interaction and selecting an antibody which interacts with the stabilised complex of the protein:protein interaction but not with each individual component of the protein:protein interaction in the absence of the other.

52. The method of claim 51, further comprising a step of determining that the antibody stabilises the protein:protein interaction.

53. Use of a binding protein as defined in any one of claims 1-20 in screening for a substance which modulates signalling through the protein:protein interaction.
